(19) United States
(12) Patent Application Publication
(10) Pub. No.: US 2024/0425492 A1
(43) Pub. Date: Dec. 26, 2024

(54) POLYMORPHS AS ERBB INHIBITORS

(71) Applicant: Black Diamond Therapeutics, Inc., Cambridge, MA (US)

(72) Inventors: Wu-Yan ZHANG, Lexington, MA (US); Reza FOROUGHI, Pacific Palisades, CA (US); Siyi JIANG, Shanghai (CN); Meiqi LI, Shanghai (CN); Juanjuan SHI, Wanghe Town (CN)

(21) Appl. No.: 18/693,733

(22) PCT Filed: Sep. 21, 2022
(86) PCT No.: PCT/US2022/044232

§ 371 Date: Mar. 20, 2024

Related U.S. Application Data
(60) Provisional application No. 63/246,451, filed on Sep. 21, 2021.

(51) Int. Cl. C07D 413/14; A61K 31/5377

(52) U.S. Cl. CPC C07D 413/14; A61K 31/5377

(57) ABSTRACT

The present disclosure provides morphic forms of Compound No. 1:

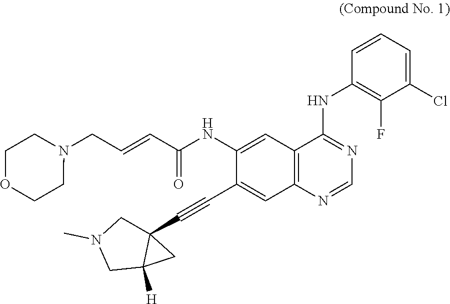 [SMILES: CN1C[C@H]2C[C@@]2(C#Cc2cc3ncnc(Nc4cccc(Cl)c4F)c3cc2NC(=O)/C=C/CN2CCOCC2)C1]

solvates thereof, hydrates thereof, and pharmaceutically acceptable salts thereof. The present disclosure also provides processes for preparing the morphic forms and uses of the morphic forms, e.g., in preventing or treating cancer.

### RELATED APPLICATION

This application claims priority to, and the benefit of, U.S. Application No. 63/246,451, filed Sep. 21, 2021, the entire content of which is incorporated herein by reference.

### BACKGROUND

Mutations affecting either the intracellular catalytic domain or extracellular ligand binding domain of an ErbB receptor can generate oncogenic activity (the ErbB protein family consists of 4 members including ErbB-1, also named epidermal growth factor receptor (EGFR) and Erb-2, also named HER2 in humans). ErbB inhibitors are a known treatment for a number of cancers.

However, not every patient is responsive satisfactorily to this treatment. Thus, there is a long-felt need in the art for new therapies that are able to address the variable responsiveness of cancer patients to known therapies. The present disclosure provides compositions and methods for preventing or treating cancer in patients with these oncogenic mutations.

### SUMMARY

In some aspects, the present disclosure provides a morphic form of Compound No. 1:

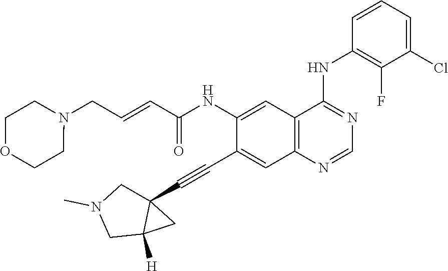 [SMILES: CN1C[C@H]2C[C@@]2(C#Cc2cc3ncnc(Nc4cccc(Cl)c4F)c3cc2NC(=O)/C=C/CN2CCOCC2)C1]

a solvate thereof, a hydrate thereof, or a pharmaceutically acceptable salt thereof.

In some aspects, the present disclosure provides a pharmaceutical composition comprising a therapeutically effective amount of a morphic form of Compound No. 1, a solvate thereof, a hydrate thereof, or a pharmaceutically acceptable salt thereof, and a pharmaceutically acceptable excipient.

In some aspects, the present disclosure provides a method of inhibiting an oncogenic variant of an ErbB receptor, comprising administering to the subject in need thereof a therapeutically effective amount of a morphic form of Compound No. 1, a solvate thereof, a hydrate thereof, or a pharmaceutically acceptable salt thereof.

In some aspects, the present disclosure provides a method of preventing or treating cancer, comprising administering to the subject in need thereof a therapeutically effective amount of a morphic form of Compound No. 1, a solvate thereof, a hydrate thereof, or a pharmaceutically acceptable salt thereof.

In some aspects, the present disclosure provides a morphic form of Compound No. 1, a solvate thereof, a hydrate thereof, or a pharmaceutically acceptable salt thereof, for use in the inhibition of an oncogenic variant of an ErbB receptor.

In some aspects, the present disclosure provides a morphic form of Compound No. 1, a solvate thereof, a hydrate thereof, or a pharmaceutically acceptable salt thereof, for use in the prevention or treatment of cancer.

In some aspects, the present disclosure provides use of a morphic form of Compound No. 1, a solvate thereof, a hydrate thereof, or a pharmaceutically acceptable salt thereof, in the manufacture of a medicament for inhibiting an oncogenic variant of an ErbB receptor.

In some aspects, the present disclosure provides use of a morphic form of Compound No. 1, a solvate thereof, a hydrate thereof, or a pharmaceutically acceptable salt thereof, in the manufacture of a medicament for preventing or treating cancer.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### BRIEF DESCRIPTIONS OF FIGURES

FIG. 1 is a graph depicting the XRPD spectrum for Form A of Compound No. 1.

FIG. 2 is a graph depicting the XRPD spectrum for Form B of Compound No. 1.

FIG. 3 is a graph depicting the XRPD spectrum for Form C of Compound No. 1.

FIG. 4 is a graph depicting the XRPD spectrum for Form D of Compound No. 1.

FIG. 5 is a graph depicting the XRPD spectrum for Form E of Compound No. 1.

FIG. 6 is a graph depicting the XRPD spectrum for Form F of Compound No. 1.

FIG. 7 is a graph depicting the XRPD spectrum for Form G of Compound No. 1.

FIG. 8 is a graph depicting the XRPD spectrum for Form H of Compound No. 1.

FIG. 9 is a graph depicting the XRPD spectrum for Form I of Compound No. 1.

FIG. 10 is a graph depicting the XRPD spectrum for Form J of Compound No. 1.

FIG. 11 is a graph depicting the XRPD spectrum for Form K of Compound No. 1.

FIG. 12 is a graph depicting the XRPD spectrum for Form L of Compound No. 1.

### DETAILED DESCRIPTION

It is understood that the term “Compound No. 1,” as used herein, refers to a compound having the following structure:

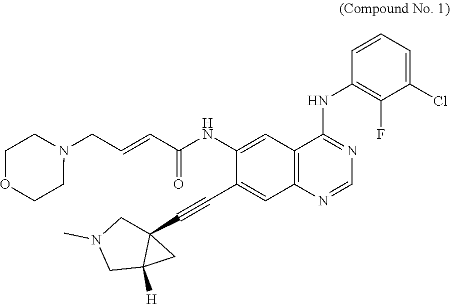 [SMILES: CN1C[C@H]2C[C@@]2(C#Cc2cc3ncnc(Nc4cccc(Cl)c4F)c3cc2NC(=O)/C=C/CN2CCOCC2)C1]

Compound No. 1 may be identified by the IUPAC name of (E)-N-(4-((3-chloro-2-fluorophenyl)amino)-7-(((1R,5S)-3-methyl-3-azabicyclo[3.1.0]hexan-1-yl)ethynyl)quinazolin-6-yl)-4-morpholinobut-2-enamide.

In some aspects, the present disclosure provides a morphic form of Compound No. 1, a solvate thereof, a hydrate thereof, or a pharmaceutically acceptable salt thereof.

In some embodiments, the morphic form is a crystalline form.

In some embodiments, the morphic form is a morphic form (e.g., crystalline form) of Compound No. 1, the solvate thereof, or the hydrate thereof.

In some embodiments, the morphic form is a morphic form (e.g., crystalline form) of Compound No. 1.

In some embodiments, the morphic form is a morphic form (e.g., crystalline form) of a solvate (e.g., heterosolvate) of Compound No. 1.

In some embodiments, the morphic form is a morphic form (e.g., crystalline form) of a hydrate of Compound No. 1.

In some embodiments, the morphic form is Form A of Compound No. 1, the solvate thereof, the hydrate thereof, or the pharmaceutically acceptable salt thereof.

In some embodiments, the morphic form is Form B of Compound No. 1, the solvate thereof, the hydrate thereof, or the pharmaceutically acceptable salt thereof.

In some embodiments, the morphic form is Form C of Compound No. 1, the solvate thereof, the hydrate thereof, or the pharmaceutically acceptable salt thereof.

In some embodiments, the morphic form is Form D of Compound No. 1, the solvate thereof, the hydrate thereof, or the pharmaceutically acceptable salt thereof.

In some embodiments, the morphic form is Form E of Compound No. 1, the solvate thereof, the hydrate thereof, or the pharmaceutically acceptable salt thereof.

In some embodiments, the morphic form is Form F of Compound No. 1, the solvate thereof, the hydrate thereof, or the pharmaceutically acceptable salt thereof.

In some embodiments, the morphic form is Form G of Compound No. 1, the solvate thereof, the hydrate thereof, or the pharmaceutically acceptable salt thereof.

In some embodiments, the morphic form is Form H of Compound No. 1, the solvate thereof, the hydrate thereof, or the pharmaceutically acceptable salt thereof.

In some embodiments, the morphic form is Form I of Compound No. 1, the solvate thereof, the hydrate thereof, or the pharmaceutically acceptable salt thereof.

In some embodiments, the morphic form is Form J of Compound No. 1, the solvate thereof, the hydrate thereof, or the pharmaceutically acceptable salt thereof.

In some embodiments, the morphic form is Form K of Compound No. 1, the solvate thereof, the hydrate thereof, or the pharmaceutically acceptable salt thereof.

In some embodiments, the morphic form is Form L of Compound No. 1, the solvate thereof, the hydrate thereof, or the pharmaceutically acceptable salt thereof.

In some aspects, the present disclosure provides a method of preparing a morphic form of Compound No. 1, the solvate thereof, the hydrate thereof, or the pharmaceutically acceptable salt thereof.

### Form A

### X-Ray Powder Diffraction (XRPD) Characterization

In some embodiments, the morphic form is Form A of Compound No. 1, the solvate thereof, the hydrate thereof, or the pharmaceutically acceptable salt thereof.

In some embodiments, the morphic form is Form A of Compound No. 1.

In some embodiments, Form A is characterized by an X-ray diffraction (“XRPD”) pattern comprising peaks at 4.8±0.2, 5.6±0.2, and 17.6±0.2°2θ (e.g., 4.8±0.1, 5.6±0.1, and 17.6±0.1°2θ (e.g., 4.8, 5.6, and 17.6°2θ)) using Cu Kα radiation.

In some embodiments, the XRPD pattern of Form A further comprises at least one peak selected from 9.6±0.2, 20.0±0.2, and 24.6±0.2°2θ (e.g., 9.6±0.1, 20.0±0.1, and 24.6±0.1°2θ (e.g., 9.6, 20.0, and 24.6°2θ)) using Cu Kα radiation.

In some embodiments, the XRPD pattern of Form A further comprises at least two peaks selected from 9.6±0.2, 20.0±0.2, and 24.6±0.2°2θ (e.g., 9.6±0.1, 20.0±0.1, and 24.6±0.1°2θ (e.g., 9.6, 20.0, and 24.6°2θ)) using Cu Kα radiation.

In some embodiments, the XRPD pattern of Form A further comprises peaks at 9.6±0.2, 20.0±0.2, and 24.6±0.2°2θ (e.g., 9.6±0.1, 20.0±0.1, and 24.6±0.1°2θ (e.g., 9.6, 20.0, and 24.6°2θ)) using Cu Kα radiation.

In some embodiments, Form A is characterized by an X-ray diffraction (“XRPD”) pattern comprising at least three peaks selected from 4.8±0.2, 5.6±0.2, 9.6±0.2, 17.6±0.2, 20.0±0.2, and 24.6±0.2°2θ (e.g., 4.8±0.1, 5.6±0.1, 9.6±0.1, 17.6±0.1, 20.0±0.1, and 24.6±0.1°2θ (e.g., 4.8, 5.6, 9.6, 17.6, 20.0, and 24.6°2θ)) using Cu Kα radiation.

In some embodiments, the XRPD pattern of Form A comprises at least four peaks selected from 4.8±0.2, 5.6±0.2, 9.6±0.2, 17.6±0.2, 20.0±0.2, and 24.6±0.2°2θ (e.g., 4.8±0.1, 5.6±0.1, 9.6±0.1, 17.6±0.1, 20.0±0.1, and 24.6±0.1°2θ (e.g., 4.8, 5.6, 9.6, 17.6, 20.0, and 24.6°2θ)) using Cu Kα radiation.

In some embodiments, the XRPD pattern of Form A comprises at least five peaks selected from 4.8±0.2, 5.6±0.2, 9.6±0.2, 17.6±0.2, 20.0±0.2, and 24.6±0.2°2θ (e.g., 4.8±0.1, 5.6±0.1, 9.6±0.1, 17.6±0.1, 20.0±0.1, and 24.6±0.1°2θ (e.g., 4.8, 5.6, 9.6, 17.6, 20.0, and 24.6°2θ)) using Cu Kα radiation.

In some embodiments, the XRPD pattern of Form A comprises peaks at 4.8±0.2, 5.6±0.2, 9.6±0.2, 17.6±0.2, 20.0±0.2, and 24.6±0.2 0 (e.g., 4.8±0.1, 5.6±0.1, 9.6±0.1, 17.6±0.1, 20.0±0.1, and 24.6±0.1°2θ (e.g., 4.8, 5.6, 9.6, 17.6, 20.0, and 24.6°2θ)) using Cu Kα radiation.

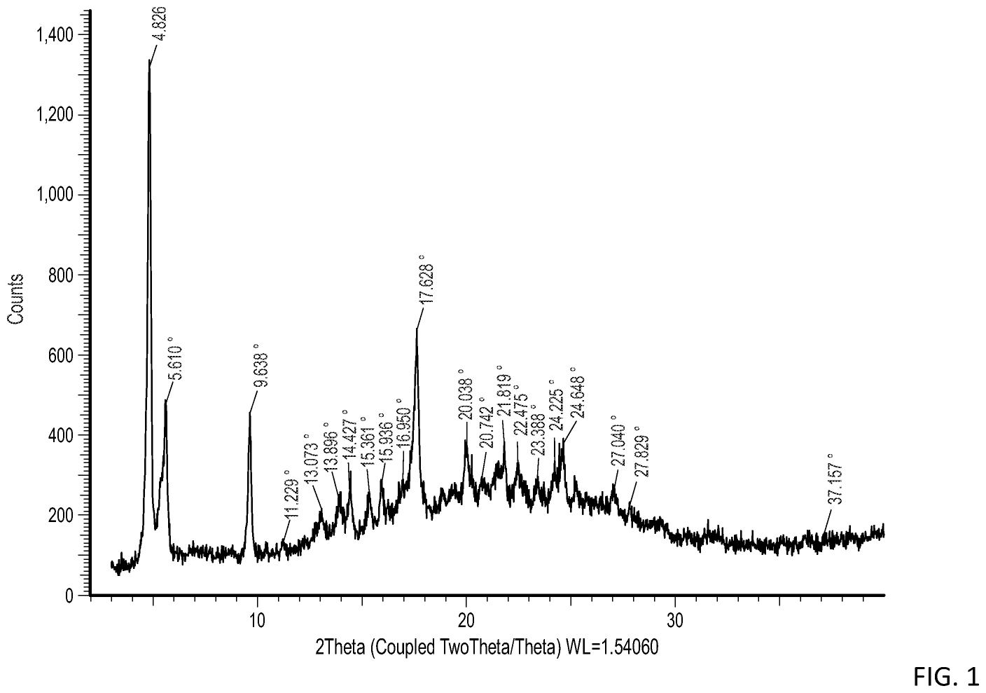

In some embodiments, Form A is characterized by an XRPD pattern substantially similar to that shown in FIG. 1.

In some embodiments, Form A is characterized by an XRPD pattern comprising peaks at approximately the positions shown in the table below. It is understood that the values in the table are approximate and subject to instrumental and experimental variations.

TABLE 1
XRPD peak list for Form A

| | | | Net | Gross | Rel. |
|---|---|---|---|---|---|
| Angle | d Value | | Intensity | Intensity | Intensity |
| 4.826° | 18.28667 | Å | 1228.33 | 1318.10 | 100.0% |
| 5.610° | 15.74120 | Å | 378.693 | 478.482 | 30.8% |
| 9.638° | 9.16911 | Å | 359.507 | 453.714 | 29.3% |
| 11.229° | 7.87356 | Å | 27.8107 | 130.954 | 2.3% |
| 13.073° | 6.76695 | Å | 80.4758 | 213.889 | 6.6% |
| 13.896° | 6.36787 | Å | 95.5909 | 239.469 | 7.8% |
| 14.427° | 6.13470 | Å | 94.0017 | 242.001 | 7.7% |
| 15.361° | 5.76358 | Å | 81.8297 | 255.322 | 7.5% |
| 15.936° | 5.55688 | Å | 96.7157 | 273.132 | 7.9% |
| 16.950° | 5.22667 | Å | 76.8431 | 269.809 | 6.39% |
| 17.628° | 5.02704 | Å | 480.999 | 660.840 | 37.5% |
| 20.038° | 4.42771 | Å | 138.461 | 370.000 | 11.3% |
| 20.742° | 4.27888 | Å | 58.2575 | 293.460 | 4.8% |
| 21.819° | 4.07011 | Å | 135.921 | 374.773 | 11.1% |
| 22.475° | 3.95268 | Å | 74.7631 | 310.463 | 6.1% |
| 23.388° | 3.80054 | Å | 47.3292 | 279.684 | 3.9% |
| 24.225° | 3.67109 | Å | 98.1923 | 331.515 | 7.8% |
| 24.648° | 3.60899 | Å | 142.917 | 377.780 | 11.6% |
| 27.040° | 3.29486 | Å | 43.3253 | 251.809 | 3.5% |
| 27.829° | 3.20327 | Å | 35.1582 | 225.962 | 2.9% |
| 37.157° | 2.41776 | Å | 31.0024 | 156.575 | 2.5% |

### Differential Scanning Calorimeter (DSC) Characterizations

In some embodiments, Form A is characterized by a DSC curve having at least one endothermic peak selected from 76±20, 141±20, 156±20, and 170±20° C. (e.g., 76±10, 141±10, 156±10, and 170±10° C. (e.g., 76±5, 141±5, 156±5, and 170±5° C. (e.g., 76±4, 141±4, 156±4, and 170±4° C. (e.g., 76±3, 141±3, 156±3, and 170±3° C. (e.g., 76±2, 141±2, 156±2, and 170±2° C. (e.g., 76±1, 141±1, 156±1, and 170±1° C. (e.g., 76±0.5, 141±0.5, 156±0.5, and 170±0.5° C.))))))).

In some embodiments, Form A is characterized by a DSC curve having at least two endothermic peaks selected from 76±20, 141±20, 156±20, and 170±20° C. (e.g., 76±10, 141±10, 156±10, and 170±10° C. (e.g., 76±5, 141±5, 156±5, and 170±5° C. (e.g., 76±4, 141±4, 156±4, and 170±4° C. (e.g., 76±3, 141±3, 156±3, and 170±3° C. (e.g., 76±2, 141±2, 156±2, and 170±2° C. (e.g., 76±1, 141±1, 156±1, and 170±1° C. (e.g., 76±0.5, 141±0.5, 156±0.5, and 170±0.5° C.))))))).

In some embodiments, Form A is characterized by a DSC curve having at least three endothermic peaks selected from 76±20, 141±20, 156±20, and 170±20° C. (e.g., 76±10, 141±10, 156±10, and 170±10° C. (e.g., 76±5, 141±5, 156±5, and 170±5° C. (e.g., 76±4, 141±4, 156±4, and 170±4° C. (e.g., 76±3, 141±3, 156±3, and 170±3° C. (e.g., 76±2, 141±2, 156±2, and 170±2° C. (e.g., 76±1, 141±1, 156±1, and 170±1° C. (e.g., 76±0.5, 141±0.5, 156±0.5, and 170±0.5° C.))))))).

In some embodiments, Form A is characterized by a DSC curve having endothermic peaks at 76±20, 141±20, 156±20, and 170±20° C. (e.g., 76±10, 141±10, 156±10, and 170±10° C. (e.g., 76±5, 141±5, 156±5, and 170±5° C. (e.g., 76±4, 141±4, 156±4, and 170±4° C. (e.g., 76±3, 141±3, 156±3, and 170±3° C. (e.g., 76±2, 141±2, 156±2, and 170±2° C. (e.g., 76±1, 141±1, 156±1, and 170±1° C. (e.g., 76±0.5, 141±0.5, 156±0.5, and 170±0.5° C.))))))).

In some embodiments, Form A is characterized by a DSC curve having an endothermic peak at 76° C. In some embodiments, Form A of Compound No. 1 is characterized by a DSC curve having an endothermic peak at 141° C. In some embodiments, Form A of Compound No. 1 is characterized by a DSC curve having an endothermic peak at 156° C. In some embodiments, Form A of Compound No. 1 is characterized by a DSC curve having an endothermic peak at 170° C.

### Thermogravimetric Analysis (TGA) Characterizations

In some embodiments, Form A shows a weight loss of approximately 1-3% between about 28±20° C. (e.g., 28±10° C. (e.g., 28±5° C. (e.g., 28±4° C. (e.g., 28±3° C. (e.g., 28±2° C. (e.g., 28±1° C. (e.g., 28±0.5° C.))))))) and about 130±20° C. (e.g., 130±10° C. (e.g., 130±5° C. (e.g., 130±4° C. (e.g., 130±3° C. (e.g., 130±2° C. (e.g., 130±1° C. (e.g., 150±0.5° C.))))))), as measured by TGA.

In some embodiments, Form A shows a weight loss of approximately 2.2% between about 28° C. and about 130° C., as measured by TGA.

### Form B

### X-Ray Powder Diffraction (XRPD) Characterization

In some embodiments, the morphic form is Form B of Compound No. 1, the solvate thereof, the hydrate thereof, or the pharmaceutically acceptable salt thereof.

In some embodiments, the morphic form is Form B of Compound No. 1.

In some embodiments, the present disclosure provides a form of Compound No. 1 (“Form B”) characterized by an X-ray diffraction (“XRPD”) pattern comprising peaks at 6.5±0.2, 16.7±0.2, and 18.6±0.2°2θ (e.g., 6.5±0.1, 16.7±0.1, and 18.6±0.1°2θ (e.g., 6.5, 16.7, and 18.6°2θ)) using Cu Kα radiation.

In some embodiments, the XRPD pattern of Form B further comprises at least one peak selected from 3.2±0.2, 13.4±0.2, and 17.7±0.2°2θ (e.g., 3.2±0.1, 13.4±0.1, and 17.7±0.1°2θ (e.g., 3.2, 13.4, and 17.7°2θ)) using Cu Kα radiation.

In some embodiments, the XRPD pattern of Form B further comprises at least two peaks selected from 3.2±0.2, 13.4±0.2, and 17.7±0.2°2θ (e.g., 3.2±0.1, 13.4±0.1, and 17.7±0.1°2θ (e.g., 3.2, 13.4, and 17.7°2θ)) using Cu Kα radiation.

In some embodiments, the XRPD pattern of Form B further comprises peaks at 3.2±0.2, 13.4±0.2, and 17.7±0.2°2θ (e.g., 3.2±0.1, 13.4±0.1, and 17.7±0.1°2θ (e.g., 3.2, 13.4, and 17.7°2θ)) using Cu Kα radiation.

In some aspects, the present disclosure provides a form of Compound No. 1 (“Form B”) characterized by an X-ray diffraction (“XRPD”) pattern comprising at least three peaks selected from 3.2±0.2, 6.5±0.2, 13.4±0.2, 16.7±0.2, 17.7±0.2, and 18.6±0.2°2θ (e.g., 3.2±0.1, 6.5±0.1, 13.4±0.1, 16.7±0.1, 17.7±0.1, and 18.6±0.1°2θ (e.g., 3.2, 6.5, 13.4, 16.7, 17.7, and 18.6°2θ)) using Cu Kα radiation.

In some embodiments, the XRPD pattern of Form B comprises at least four peaks selected from 3.2±0.2, 6.5±0.2, 13.4±0.2, 16.7±0.2, 17.7±0.2, and 18.6±0.2°2θ (e.g., 3.2±0.1, 6.5±0.1, 13.4±0.1, 16.7±0.1, 17.7±0.1, and 18.6±0.1°2θ (e.g., 3.2, 6.5, 13.4, 16.7, 17.7, and 18.6°2θ)) using Cu Kα radiation.

In some embodiments, the XRPD pattern of Form B comprises at least five peaks selected from 3.2±0.2, 6.5±0.2, 13.4±0.2, 16.7±0.2, 17.7±0.2, and 18.6±0.2°2θ (e.g., 3.2±0.1, 6.5±0.1, 13.4±0.1, 16.7±0.1, 17.7±0.1, and 18.6±0.1°2θ (e.g., 3.2, 6.5, 13.4, 16.7, 17.7, and 18.6°2θ)) using Cu Kα radiation.

In some embodiments, the XRPD pattern of Form B comprises peaks at 3.2±0.2, 6.5±0.2, 13.4±0.2, 16.7±0.2, 17.7±0.2, and 18.6±0.2°2θ (e.g., 3.2±0.1, 6.5±0.1, 13.4±0.1, 16.7±0.1, 17.7±0.1, and 18.6±0.1°2θ (e.g., 3.2, 6.5, 13.4, 16.7, 17.7, and 18.6°2θ)) using Cu Kα radiation.

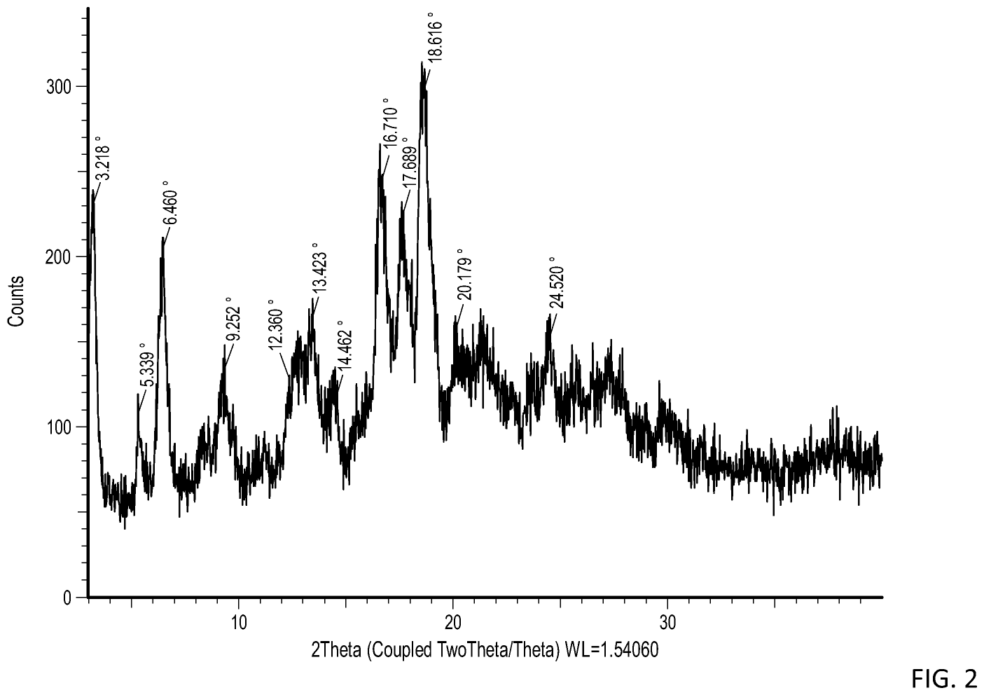

In some embodiments, Form B is characterized by an XRPD pattern substantially similar to that shown in FIG. 2.

In some embodiments, Form B is characterized by an XRPD pattern comprising peaks at approximately the positions shown in the table below. It is understood that the values in the table are approximate and subject to instrumental and experimental variations.

TABLE 2
XRPD peak list for Form B

| | | | Net | Gross | Rel |
|---|---|---|---|---|---|
| Angle | d Value | | Intensity | Intensity | Intensity |
| 3.218° | 27.43768 | Å | 97.1737 | 230.199 | 51.6% |
| 5.339° | 16.53828 | Å | 51.3732 | 108.372 | 27.3% |
| 6.460° | 13.67139 | Å | 142.552 | 204.055 | 75.7% |
| 9.252° | 9.55124 | Å | 62.9430 | 130.725 | 33.4% |
| 12.360° | 7.16525 | Å | 49.9792 | 128.380 | 26.5% |
| 13.423° | 6.50100 | Å | 81.3251 | 162.990 | 43.2% |
| 14.462° | 6.11985 | Å | 32.0142 | 113.000 | 17.0% |
| 16.710° | 5.30119 | Å | 147.920 | 246.209 | 78.5% |
| 17.689° | 5.00999 | Å | 121.028 | 225.974 | 64.2% |
| 18.616° | 4.76251 | Å | 188.420 | 296.540 | 100.0% |
| 20.179° | 4.39696 | Å | 49.6301 | 158.829 | 26.3% |
| 24.520° | 3.62757 | Å | 47.4184 | 153.251 | 25.2% |

### Differential Scanning Calorimeter (DSC) Characterizations

In some embodiments, Form B of Compound No. 1 is characterized by a DSC curve having at least one endothermic peak selected from 155±20 and 176±20° C. (e.g., 155±10 and 176±10° C. (e.g., 155±5 and 176±5° C. (e.g., 155±4 and 176±4° C. (e.g., 155±3 and 176±3° C. (e.g., 155±2 and 176±2° C. (e.g., 155±1 and 176±1° C. (e.g., 155±0.5 and 176±0.5° C.))))))).

In some embodiments, Form B of Compound No. 1 is characterized by a DSC curve having endothermic peaks at 155±20 and 176±20° C. (e.g., 155±10 and 176±10° C. (e.g., 155±5 and 176±5° C. (e.g., 155±4 and 176±4° C. (e.g., 155±3 and 176±3° C. (e.g., 155±2 and 176±2° C. (e.g., 155±1 and 176±1° C. (e.g., 155±0.5 and 176±0.5° C.))))))).

In some embodiments, Form B of Compound No. 1 is characterized by a DSC curve having an endothermic peak at 155° C. In some embodiments, Form B of Compound No. 1 is characterized by a DSC curve having an endothermic peak at 176° C.

### Thermogravimetric Analysis (TGA) Characterizations

In some embodiments, Form B of Compound No. 1 shows a weight loss of approximately 1-3% between about 35±20° C. (e.g., 35±10° C. (e.g., 35±5° C. (e.g., 35±4° C. (e.g., 35±3° C. (e.g., 35±2° C. (e.g., 35±1° C. (e.g., 35±0.5° C.))))))) and about 150±20° C. (e.g., 150±10° C. (e.g., 150±5° C. (e.g., 150±4° C. (e.g., 150±3° C. (e.g., 150±2° C. (e.g., 150±1° C. (e.g., 150±0.5° C.))))))), as measured by TGA.

In some embodiments, Form B of Compound No. 1 shows a weight loss of approximately 1.5% between about 35° C. and about 150° C., as measured by TGA.

### Form C

### X-Ray Powder Diffraction (XRPD) Characterization

In some embodiments, the morphic form is Form C of Compound No. 1, the solvate thereof, the hydrate thereof, or the pharmaceutically acceptable salt thereof.

In some embodiments, the morphic form is Form C of Compound No. 1.

In some embodiments, the present disclosure provides a form of Compound No. 1 (“Form C”) characterized by an X-ray diffraction (“XRPD”) pattern comprising peaks at 16.1±0.2, 16.7±0.2, and 19.1±0.2°2θ (e.g., 16.1±0.1, 16.7±0.1, and 19.1±0.1°2θ (e.g., 16.1, 16.7, and 19.1°2θ)) using Cu Kα radiation.

In some embodiments, the XRPD pattern of Form C further comprises at least one peak selected from 4.0±0.2, 12.6±0.2, and 14.1±0.2°2θ (e.g., 4.0±0.1, 12.6±0.1, and 14.1±0.1°2θ (e.g., 4.0, 12.6, and 14.1°2θ)) using Cu Kα radiation.

In some embodiments, the XRPD pattern of Form C further comprises at least two peaks selected from 4.0±0.2, 12.6±0.2, and 14.1±0.2°2θ (e.g., 4.0±0.1, 12.6±0.1, and 14.1±0.1°2θ (e.g., 4.0, 12.6, and 14.1°2θ)) using Cu Kα radiation.

In some embodiments, the XRPD pattern of Form C further comprises peaks at 4.0±0.2, 12.6±0.2, and 14.1±0.2°2θ (e.g., 4.0±0.1, 12.6±0.1, and 14.1±0.1°2θ (e.g., 4.0, 12.6, and 14.1°2θ)) using Cu Kα radiation.

In some aspects, the present disclosure provides a form of Compound No. 1 (“Form C”) characterized by an X-ray diffraction (“XRPD”) pattern comprising at least three peaks selected from 4.0±0.2, 12.6±0.2, 14.1±0.2, 16.1±0.2, 16.7±0.2, and 19.1±0.2°2θ (e.g., 4.0±0.1, 12.6±0.1, 14.1±0.1, 16.1±0.1, 16.7±0.1, and 19.1±0.1°2θ (e.g., 4.0, 12.6, 14.1, 16.1, 16.7, and 19.1°2θ)) using Cu Kα radiation.

In some embodiments, the XRPD pattern of Form C comprises at least four peaks selected from 4.0±0.2, 12.6±0.2, 14.1±0.2, 16.1±0.2, 16.7±0.2, and 19.1±0.2°2θ (e.g., 4.0±0.1, 12.6±0.1, 14.1±0.1, 16.1±0.1, 16.7±0.1, and 19.1±0.1°2θ (e.g., 4.0, 12.6, 14.1, 16.1, 16.7, and 19.1°2θ)) using Cu Kα radiation.

In some embodiments, the XRPD pattern of Form C comprises at least five peaks selected from 4.0±0.2, 12.6±0.2, 14.1±0.2, 16.1±0.2, 16.7±0.2, and 19.1±0.2°2θ (e.g., 4.0±0.1, 12.6±0.1, 14.1±0.1, 16.1±0.1, 16.7±0.1, and 19.1±0.1°2θ (e.g., 4.0, 12.6, 14.1, 16.1, 16.7, and 19.1°2θ)) using Cu Kα radiation.

In some embodiments, the XRPD pattern of Form C comprises peaks at 4.0±0.2, 12.6±0.2, 14.1±0.2, 16.1±0.2, 16.7±0.2, and 19.1±0.2°2θ (e.g., 4.0±0.1, 12.6±0.1, 14.1±0.1, 16.1±0.1, 16.7±0.1, and 19.1±0.1°2θ (e.g., 4.0, 12.6, 14.1, 16.1, 16.7, and 19.1°2θ)) using Cu Kα radiation.

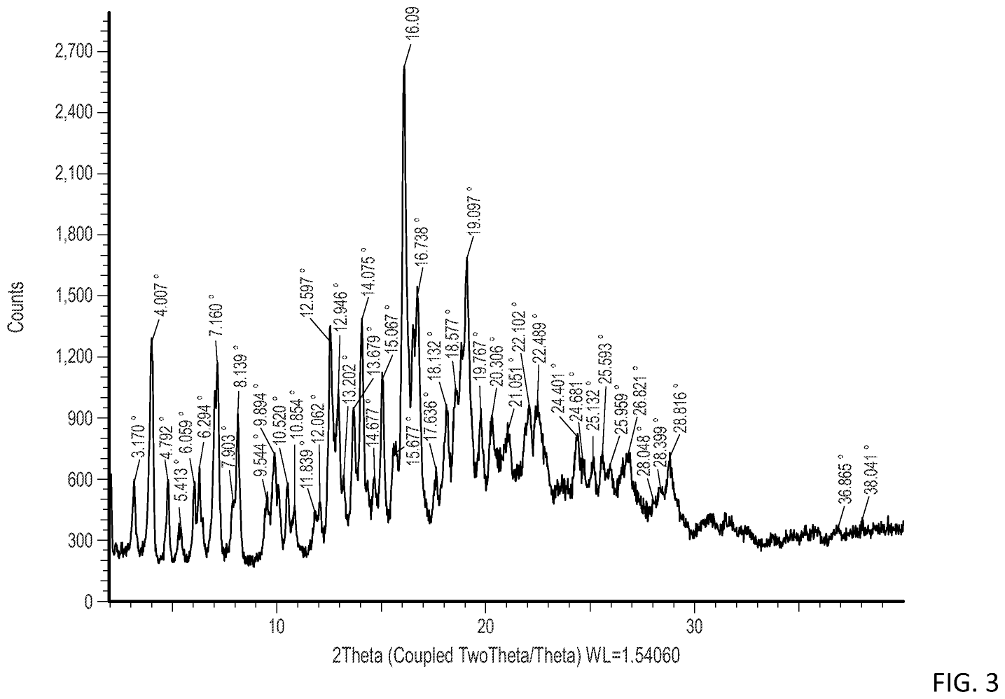

In some embodiments, Form C is characterized by an XRPD pattern substantially similar to that shown in FIG. 3.

In some embodiments, Form C is characterized by an XRPD pattern comprising peaks at approximately the positions shown in the table below. It is understood that the values in the table are approximate and subject to instrumental and experimental variations.

TABLE 3
XRPD peak list for Form C

| | | | Net | Gross | Rel. |
|---|---|---|---|---|---|
| Angle | d Value | | Intensity | Intensity | Intensity |
| 3.170° | 27.84797 | Å | 357.930 | 582.542 | 36.1% |
| 4.007° | 22.03308 | Å | 1064.20 | 1271.82 | 47.8% |
| 4.792° | 18.42484 | Å | 387.461 | 589.584 | 17.4% |
| 5.413° | 16.31162 | Å | 153.789 | 357.464 | 6.9% |
| 6.059° | 14.57441 | Å | 367.209 | 572.881 | 16.5% |
| 6.284° | 14.03053 | Å | 447.047 | 653.667 | 20.1% |
| 7.180° | 12.33580 | Å | 948.205 | 1154.38 | 42.7% |
| 7.903° | 11.17780 | Å | 292.347 | 491.842 | 13.2% |
| 8.139° | 10.85462 | Å | 703.897 | 800.093 | 31.7% |
| 9.544° | 9.25958 | Å | 286.580 | 489.354 | 12.9% |
| 9.694° | 8.93263 | Å | 507.211 | 719.206 | 23.8% |
| 10.520° | 8.40217 | Å | 345.232 | 570.588 | 15.5% |
| 10.854° | 8.14451 | Å | 223.889 | 454.525 | 10.1% |
| 11.838° | 7.48925 | Å | 171.484 | 432.989 | 7.7% |
| 12.062° | 7.33153 | Å | 205.918 | 479.445 | 9.3% |
| 12.597° | 7.02155 | Å | 969.853 | 1270.08 | 43.7% |
| 12.948° | 8.83288 | Å | 709.879 | 1025.93 | 32.0% |
| 13.202° | 6.70074 | Å | 287.138 | 614.000 | 12.9% |
| 13.679° | 6.48821 | Å | 598.928 | 944.172 | 27.0% |
| 14.075° | 6.28717 | Å | 1023.34 | 5381.85 | 46.19% |
| 14.877° | 8.03065 | Å | 231.660 | 607.779 | 10.4% |
| 15.067° | 5.87541 | Å | 707.199 | 1092.61 | 31.8% |
| 15.677° | 5.64830 | Å | 330.618 | 727.438 | 14.9% |
| 16.098° | 5.50138 | Å | 2221.33 | 2623.81 | 100.0% |
| 16.738° | 5.29250 | Å | 1097.38 | 1504.95 | 48.4% |
| 17.836° | 5.02504 | Å | 224.212 | 644.759 | 10.1% |
| 18.332° | 4.88851 | Å | 510.222 | 952.564 | 23.0% |
| 18.577° | 4.77248 | Å | 569.958 | 1029.67 | 25.7% |
| 19.097° | 4.64356 | Å | 1205.34 | 1682.60 | 54.3% |
| 19.767° | 4.48775 | Å | 428.911 | 928.136 | 19.4% |
| 20.306° | 4.36978 | Å | 401.569 | 909.55 | 18.1% |
| 21.053° | 4.21681 | Å | 311.749 | 831.092 | 14.0% |
| 22.102° | 4.01861 | Å | 429.315 | 955.000 | 19.3% |
| 22.408° | 3.95034 | Å | 451.995 | 977.165 | 20.3% |
| 24.401° | 3.64496 | Å | 290.060 | 805.974 | 13.1% |
| 24.683° | 3.60417 | Å | 150.508 | 663.501 | 6.8% |
| 25.132° | 3.54054 | Å | 159.741 | 866.354 | 7.2% |
| 25.593° | 3.47784 | Å | 233.972 | 731.913 | 10.5% |
| 25.958° | 3.42968 | Å | 178.644 | 669.153 | 8.1% |
| 26.823° | 3.32127 | Å | 258.846 | 722.823 | 11.8% |
| 28.048° | 3.17880 | Å | 72.8125 | 491.757 | 3.3% |
| 28.399° | 3.14030 | Å | 128.289 | 533.637 | 5.8% |
| 28.816° | 3.09575 | Å | 319.692 | 707.389 | 14.4% |
| 36.865° | 2.43623 | Å | 46.6316 | 353.792 | 2.1% |
| 38.041° | 2.36358 | Å | 69.9863 | 402.336 | 3.2% |

### Differential Scanning Calorimeter (DSC) Characterizations

In some embodiments, Form C of Compound No. 1 is characterized by a DSC curve having an endothermic peak at 181±20° C. (e.g., 181±10° C. (e.g., 181±5° C. (e.g., 181±4° C. (e.g., 181±3° C. (e.g., 181±2° C. (e.g., 181±1° C. (e.g., 181±0.5° C.))))))).

In some embodiments, Form C of Compound No. 1 is characterized by a DSC curve having an endothermic peak at 181° C.

### Thermogravimetric Analysis (TGA) Characterizations

In some embodiments, Form C of Compound No. 1 shows a weight loss of approximately 1-3% between about 28±20° C. (e.g., 28±10° C. (e.g., 28±5° C. (e.g., 28±4° C. (e.g., 28±3° C. (e.g., 28±2° C. (e.g., 28±1° C. (e.g., 28±0.5° C.))))))) and about 170±20° C. (e.g., 170±10° C. (e.g., 170±5° C. (e.g., 170±4° C. (e.g., 170±3° C. (e.g., 170±2° C. (e.g., 170±1° C. (e.g., 170±0.5° C.))))))), as measured by TGA.

In some embodiments, Form C of Compound No. 1 shows a weight loss of approximately 1.3% between about 28° C. and about 170° C., as measured by TGA.

### Form D

### X-Ray Powder Diffraction (XRPD) Characterization

In some embodiments, the morphic form is Form D of Compound No. 1, the solvate thereof, the hydrate thereof, or the pharmaceutically acceptable salt thereof.

In some embodiments, the morphic form is Form D of the dioxane solvate of Compound No. 1.

In some embodiments, the morphic form is Form D of Compound No. 1.

In some embodiments, the present disclosure provides a form of Compound No. 1 (“Form D”) characterized by an X-ray diffraction (“XRPD”) pattern comprising peaks at 15.3±0.2, 19.3±0.2, and 23.5±0.2°2θ (e.g., 5.3±0.1, 19.3±0.1, and 23.5±0.1°2θ (e.g., 5.3, 19.3, and 23.5°2θ)) using Cu Kα radiation.

In some embodiments, the XRPD pattern of Form D further comprises at least one peak selected from 5.4±0.2, 9.7±0.2, and 18.8±0.2°2θ (e.g., 5.4±0.1, 9.7±0.1, and 18.8±0.1°2θ (e.g., 5.4, 9.7, and 18.8°2θ)) using Cu Kα radiation.

In some embodiments, the XRPD pattern of Form D further comprises at least two peaks selected from 5.4±0.2, 9.7±0.2, and 18.8±0.2°2θ (e.g., 5.4±0.1, 9.7±0.1, and 18.8±0.1°2θ (e.g., 5.4, 9.7, and 18.8°2θ)) using Cu Kα radiation.

In some embodiments, the XRPD pattern of Form D further comprises peaks at 5.4±0.2, 9.7±0.2, and 18.8±0.2°2θ (e.g., 5.4±0.1, 9.7±0.1, and 18.8±0.1°2θ (e.g., 5.4, 9.7, and 18.8°2θ)) using Cu Kα radiation.

In some aspects, the present disclosure provides a form of Compound No. 1 (“Form D”) characterized by an X-ray diffraction (“XRPD”) pattern comprising at least three peaks selected from 5.4±0.2, 9.7±0.2, 15.3±0.2, 18.8±0.2, 19.3±0.2, and 23.5±0.2°2θ (e.g., 5.4±0.1, 9.7±0.1, 15.3±0.1, 18.8±0.1, 19.3±0.1, and 23.5±0.1°2θ (e.g., 5.4, 9.7, 15.3, 18.8, 19.3, and 23.5°2θ)) using Cu Kα radiation.

In some embodiments, the XRPD pattern of Form D comprises at least four peaks selected from 5.4±0.2, 9.7±0.2, 15.3±0.2, 18.8±0.2, 19.3±0.2, and 23.5±0.2°2θ (e.g., 5.4±0.1, 9.7±0.1, 15.3±0.1, 18.8±0.1, 19.3±0.1, and 23.5±0.1°2θ (e.g., 5.4, 9.7, 15.3, 18.8, 19.3, and 23.5°2θ)) using Cu Kα radiation.

In some embodiments, the XRPD pattern of Form D comprises at least five peaks selected from 5.4±0.2, 9.7±0.2, 15.3±0.2, 18.8±0.2, 19.3±0.2, and 23.5±0.2°2θ (e.g., 5.4±0.1, 9.7±0.1, 15.3±0.1, 18.8±0.1, 19.3±0.1, and 23.5±0.1°2θ (e.g., 5.4, 9.7, 15.3, 18.8, 19.3, and 23.5°2θ)) using Cu Kα radiation.

In some embodiments, the XRPD pattern of Form D comprises peaks at 5.4±0.2, 9.7±0.2, 15.3±0.2, 18.8±0.2, 19.3±0.2, and 23.5±0.2°2θ (e.g., 5.4±0.1, 9.7±0.1, 15.3±0.1, 18.8±0.1, 19.3±0.1, and 23.5±0.1°2θ (e.g., 5.4, 9.7, 15.3, 18.8, 19.3, and 23.5°2θ)) using Cu Kα radiation.

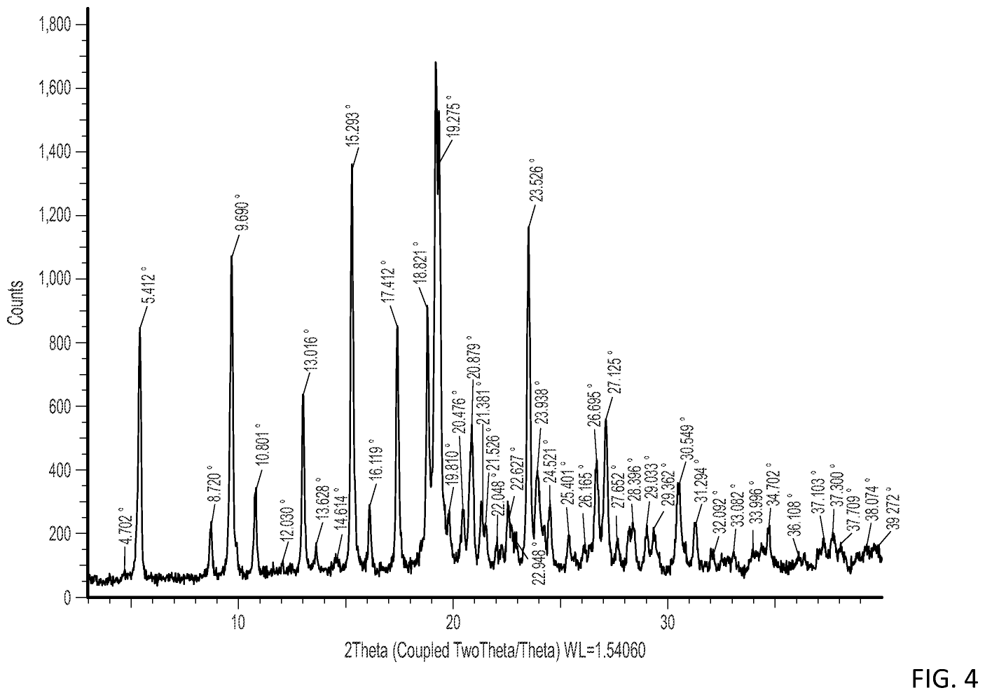

In some embodiments, Form D is characterized by an XRPD pattern substantially similar to that shown in FIG. 4.

In some embodiments, Form D is characterized by an XRPD pattern comprising peaks at approximately the positions shown in the table below. It is understood that the values in the table are approximate and subject to instrumental and experimental variations.

TABLE 4
XRPD peak list for Form D

| | | | Net | Gross | Rel. |
|---|---|---|---|---|---|
| Angle | d Value | | Intensity | Intensity | Intensity |
| 4.702° | 18.77746 | Å | 30.1557 | 84.3213 | 2.4% |
| 5.412° | 16.31821 | Å | 784.872 | 843.169 | 82.5% |
| 8.720° | 10.13200 | Å | 148.835 | 213.338 | 11.9% |
| 9.680° | 9.12049 | Å | 898.716 | 1068.22 | 79.53% |
| 10.80° | 8.18440 | Å | 255.468 | 329.066 | 20.3% |
| 12.030° | 7.35071 | Å | 23.7614 | 102.958 | 1.9% |
| 33.016° | 6.79815 | Å | 554.186 | 634.824 | 44.1% |
| 33.628° | 6.49250 | Å | 81.9099 | 563.217 | 6.5% |
| 14.614° | 6.05867 | Å | 38.5626 | 123.848 | 3.1% |
| 15.293° | 5.78923 | Å | 1255.81 | 1341.87 | 100.0% |
| 16.119° | 5.49424 | Å | 201.084 | 285.292 | 16.0% |
| 37.432° | 5.08899 | Å | 762.908 | 850.085 | 60.836 |
| 18.821° | 4.71119 | Å | 801.267 | 802.764 | 63.89% |
| 19.275° | 4.80112 | Å | 1248.63 | 1354.41 | 99.4% |
| 19.810° | 4.47800 | Å | 152.362 | 281.000 | 12.1% |
| 20.476° | 4.33385 | Å | 156.036 | 264.280 | 12.4% |
| 20.878° | 4.25121 | Å | 425.760 | 533.093 | 33.9% |
| 21.381° | 4.15254 | Å | 144.283 | 247.774 | 11.5% |
| 21.526° | 4.12485 | Å | 120.235 | 222.228 | 9.8% |
| 22.048° | 4.02831 | Å | 65.4613 | 564.482 | 5.2% |
| 22.627° | 3.92653 | Å | 144.130 | 247.670 | 11.58% |
| 22.948° | 3.87236 | Å | 70.7758 | 175.868 | 5.6% |
| 23.526° | 3.77844 | Å | 1055.98 | 1161.15 | 84.1% |
| 23.938° | 3.7144 | Å | 291.862 | 385.549 | 23.2% |
| 24.521° | 3.62739 | Å | 176.481 | 275.692 | 14.1% |
| 25.401° | 3.50372 | Å | 101.968 | 194.037 | 8.1% |
| 26.165° | 3.40314 | Å | 64.3114 | 162.588 | 5.1% |
| 26.695° | 3.33666 | Å | 326.096 | 425.877 | 26.0% |
| 27.125° | 3.28478 | Å | 457.765 | 557.060 | 36.5% |
| 27.652° | 3.22342 | Å | 77.1460 | 173.784 | 6.19% |
| 28.396° | 3.14060 | Å | 139.586 | 232.375 | 11.1% |
| 28.033° | 3.07306 | Å | 536.763 | 224.889 | 10.9% |
| 29.362° | 3.03945 | Å | 530.993 | 215.569 | 10.4% |
| 30.549° | 2.92397 | Å | 269.831 | 354.142 | 21.5% |
| 31.284° | 2.85600 | Å | 132.317 | 222.125 | 10.5% |
| 32.092° | 2.78634 | Å | 29.6543 | 120.287 | 2.4% |
| 33.082° | 2.70566 | Å | 56.7468 | 140.525 | 4.598 |
| 33.996° | 2.63499 | Å | 47.7918 | 131.253 | 3.8% |
| 34.702° | 2.58293 | Å | 515.373 | 201.136 | 9.2% |
| 36.108° | 2.48555 | Å | 43.9781 | 533.369 | 3.5% |
| 37.103° | 2.42113 | Å | 54.6017 | 145.826 | 4.3% |
| 37.300° | 2.40882 | Å | 91.8750 | 182.700 | 7.3% |
| 37.709° | 2.38362 | Å | 112.413 | 202.000 | 9.0% |
| 38.074° | 2.36158 | Å | 79.5787 | 166.722 | 6.3% |
| 39.272° | 2.28229 | Å | 53.2502 | 155.000 | 4.29% |
| 39.670° | 2.27016 | Å | 38.1215 | 148.239 | 3.0% |

### Differential Scanning Calorimeter (DSC) Characterizations

In some embodiments, Form D of Compound No. 1 is characterized by a DSC curve having at least one endothermic peak selected from 128±20, 149±20, and 175±20° C. (e.g., 128±10, 149±10, and 175±10° C. (e.g., 128±5, 149±5, and 175±5° C. (e.g., 128±4, 149±4, and 175±4° C. (e.g., 128±3, 149±3, and 175±3° C. (e.g., 128±2, 149±2, and 175±2° C. (e.g., 128±1, 149±1, and 175±1° C. (e.g., 128±0.5, 149±0.5, and 175±0.5° C.))))))).

In some embodiments, Form D of Compound No. 1 is characterized by a DSC curve having at least two endothermic peaks selected from 128±20, 149±20, and 175±20° C. (e.g., 128±10, 149±10, and 175±10° C. (e.g., 128±5, 149±5, and 175±5° C. (e.g., 128±4, 149±4, and 175±4° C. (e.g., 128±3, 149±3, and 175±3° C. (e.g., 128±2, 149±2, and 175±2° C. (e.g., 128±1, 149±1, and 175±3° C. (e.g., 128±0.5, 149±0.5, and 175±0.5° C.))))))).

In some embodiments, Form D of Compound No. 1 is characterized by a DSC curve having endothermic peaks at 128±20, 149±20, and 175±20° C. (e.g., 128±10, 149±10, and 175±10° C. (e.g., 128±5, 149±5, and 175±5° C. (e.g., 128±4, 149±4, and 175±4° C. (e.g., 128±3, 149±3, and 175±3° C. (e.g., 128±2, 149±2, and 175±2° C. (e.g., 128±1, 149±1, and 175±1° C. (e.g., 128±0.5, 149±0.5, and 175±0.5° C.))))))).

In some embodiments, Form D of Compound No. 1 is characterized by a DSC curve having an endothermic peak at 128° C. In some embodiments, Form D of Compound No. 1 is characterized by a DSC curve having an endothermic peak at 149° C. In some embodiments, Form D of Compound No. 1 is characterized by a DSC curve having an endothermic peak at 175° C.

### Thermogravimetric Analysis (IGA) Characterizations

In some embodiments, Form D of Compound No. 1 shows a weight loss of approximately 12-15% between about 33±20° C. (e.g., 33±10° C. (e.g., 33±5° C. (e.g., 33±4° C. (e.g., 33±3° C. (e.g., 33±2° C. (e.g., 33±1° C. (e.g., 33±0.5° C.))))))) and about 185±20° C. (e.g., 185±10° C. (e.g., 185±5° C. (e.g., 185±4° C. (e.g., 185±3° C. (e.g., 185±2° C. (e.g., 185±1° C. (e.g., 185±0.5° C.))))))), as measured by TGA.

In some embodiments, Form D of Compound No. 1 shows a weight loss of approximately 13.8% between about 33° C. and about 185° C., as measured by TGA.

### Form E

### X-Ray Powder Diffraction (XRPD) Characterization

In some embodiments, the morphic form is Form E of Compound No. 1, the solvate thereof, the hydrate thereof, or the pharmaceutically acceptable salt thereof.

In some embodiments, the morphic form is Form E of Compound No. 1.

In some embodiments, the present disclosure provides a form of Compound No. 1 (“Form E”) characterized by an X-ray diffraction (“XRPD”) pattern comprising peaks at 7.4±0.2, 15.8±0.2, and 16.3±0.2°2θ (e.g., 7.4±0.1, 15.8±0.1, and 16.3±0.1°2θ (e.g., 7.4, 15.8, and 16.3°2θ)) using Cu Kα radiation.

In some embodiments, the XRPD pattern of Form E further comprises at least one peak selected from 13.3±0.2, 19.2±0.2, and 22.4±0.2°2θ (e.g., 13.3±0.1, 19.2±0.1, and 22.4±0.1°2θ (e.g., 13.3, 19.2, and 22.4°2θ)) using Cu Kα radiation.

In some embodiments, the XRPD pattern of Form E further comprises at least two peaks selected from 13.3±0.2, 19.2±0.2, and 22.4±0.2°2θ (e.g., 13.3±0.1, 19.2±0.1, and 22.4±0.1°2θ (e.g., 13.3, 19.2, and 22.4°2θ)) using Cu Kα radiation.

In some embodiments, the XRPD pattern of Form E further comprises peaks at 13.3±0.2, 19.2±0.2, and 22.4±0.2°2θ (e.g., 13.3±0.1, 19.2±0.1, and 22.4±0.1°2θ (e.g., 13.3, 19.2, and 22.4°2θ)) using Cu Kα radiation.

In some aspects, the present disclosure provides a form of Compound No. 1 (“Form E”) characterized by an X-ray diffraction (“XRPD”) pattern comprising at least three peaks selected from 7.4±0.2, 13.3±0.2, 15.8±0.2, 16.3±0.2, 19.2±0.2, and 22.4±0.2°2θ (e.g., 7.4±0.1, 13.3±0.1, 15.8±0.1, 16.3±0.1, 19.2±0.1, and 22.4±0.1°2θ (e.g., 7.4, 13.3, 15.8, 16.3, 19.2, and 22.4°2θ)) using Cu Kα radiation.

In some embodiments, the XRPD pattern of Form E comprises at least four peaks selected from 7.4±0.2, 13.3±0.2, 15.8±0.2, 16.3±0.2, 19.2±0.2, and 22.4±0.2°2θ (e.g., 7.4±0.1, 13.3±0.1, 15.8±0.1, 16.3±0.1, 19.2±0.1, and 22.4±0.1°2θ (e.g., 7.4, 13.3, 15.8, 16.3, 19.2, and 22.4°2θ)) using Cu Kα radiation.

In some embodiments, the XRPD pattern of Form E comprises at least five peaks selected from 7.4±0.2, 13.3±0.2, 15.8±0.2, 16.3±0.2, 19.2±0.2, and 22.4±0.2°2θ (e.g., 7.4±0.1, 13.3±0.1, 15.8±0.1, 16.3±0.1, 19.2±0.1, and 22.4±0.1°2θ (e.g., 7.4, 13.3, 15.8, 16.3, 19.2, and 22.4°2θ)) using Cu Kα radiation.

In some embodiments, the XRPD pattern of Form E comprises peaks at 7.4±0.2, 13.3±0.2, 15.8±0.2, 16.3±0.2, 19.2±0.2, and 22.4±0.2°2θ (e.g., 7.4±0.1, 13.3±0.1, 15.8±0.1, 16.3±0.1, 19.2±0.1, and 22.4±0.1°2θ (e.g., 7.4, 13.3, 15.8, 16.3, 19.2, and 22.4°2θ)) using Cu Kα radiation.

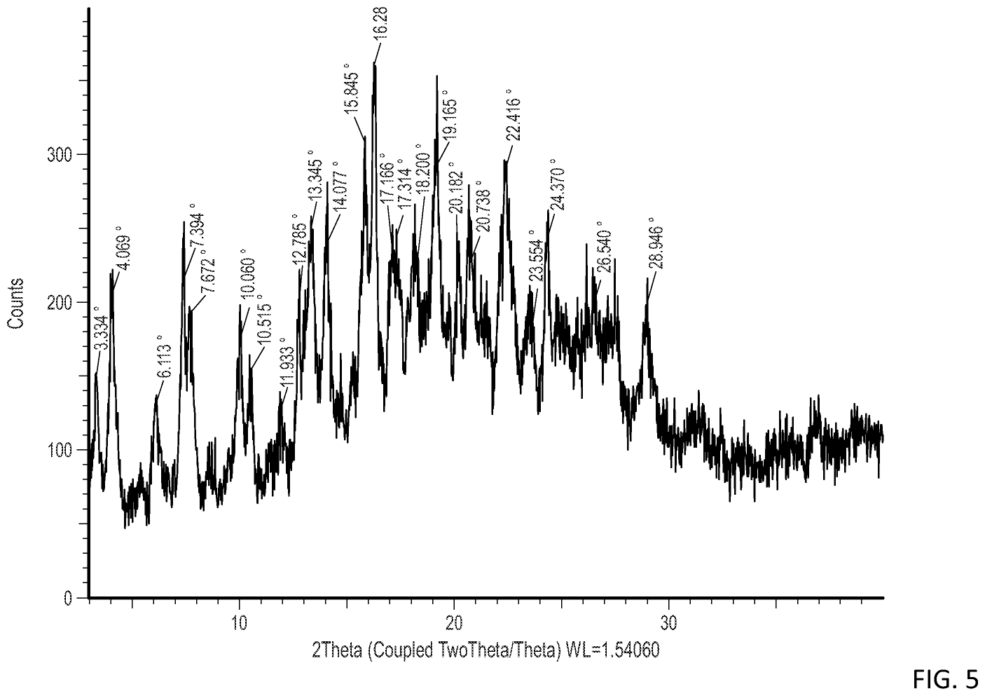

In some embodiments, Form E is characterized by an XRPD pattern substantially similar to that shown in FIG. 5.

In some embodiments, Form E is characterized by an XRPD pattern comprising peaks at approximately the positions shown in the table below. It is understood that the values in the table are approximate and subject to instrumental and experimental variations.

TABLE 5
XRPD peak list for Form E

| | | | Net | Gross | Rel. |
|---|---|---|---|---|---|
| Angle | d Value | | Intensity | Intensity | Intensity |
| 3.334° | 26.48068 | Å | 60.7883 | 149.952 | 27.0% |
| 4.069° | 21.69794 | Å | 129.288 | 205.142 | 57.4% |
| 6.113° | 14.44714 | Å | 71.1827 | 131.674 | 31.6% |
| 7.394° | 11.94611 | Å | 146.949 | 215.031 | 65.3% |
| 7.672° | 11.51440 | Å | 123.041 | 191.944 | 54.7% |
| 10.060° | 8.78552 | Å | 103.930 | 176.188 | 46.2% |
| 10.515° | 8.40646 | Å | 80.9248 | 153.318 | 35.9% |
| 11.933° | 7.41078 | Å | 37.9399 | 127.250 | 16.9% |
| 12.785° | 6.91863 | Å | 99.9288 | 200.068 | 44.4% |
| 13.345° | 6.62961 | Å | 144.086 | 249.832 | 64.0% |
| 14.077° | 6.28614 | Å | 128.605 | 239.890 | 57.1% |
| 15.845° | 5.58869 | Å | 179.813 | 308.546 | 79.9% |
| 16.280° | 5.44020 | Å | 225.129 | 360.121 | 100.0% |
| 17.166° | 5.16133 | Å | 83.9883 | 229.500 | 37.3% |
| 17.314° | 5.11770 | Å | 96.5873 | 243.560 | 42.9% |
| 18.200° | 4.87036 | Å | 61.3576 | 215.369 | 27.3% |
| 19.165° | 4.62726 | Å | 133.262 | 291.536 | 59.2% |
| 20.182° | 4.39641 | Å | 59.8528 | 219.278 | 26.6% |
| 20.738° | 4.27985 | Å | 66.4025 | 225.741 | 29.5% |
| 22.416° | 3.96312 | Å | 138.858 | 290.744 | 61.7% |
| 23.554° | 3.77410 | Å | 32.1467 | 172.790 | 14.3% |
| 24.370° | 3.64949 | Å | 106.952 | 244.421 | 47.5% |
| 26.540° | 3.35587 | Å | 65.8081 | 199.584 | 29.2% |
| 28.946° | 3.08211 | Å | 81.7087 | 196.469 | 36.3% |

### Differential Scanning Calorimeter (DSC) Characterizations

In some embodiments, Form E of Compound No. 1 is characterized by a DSC curve having an endothermic peak at 173±20° C. (e.g., 173±10° C. (e.g., 173±5° C. (e.g., 173±4° C. (e.g., 173±3° C. (e.g., 173±2° C. (e.g., 173±1° C. (e.g., 173±0.5° C.))))))).

In some embodiments, Form E of Compound No. 1 is characterized by a DSC curve having an endothermic peak at 173° C.

### Thermogravimetric Analysis (TGA) Characterizations

In some embodiments, Form E of Compound No. 1 shows a weight loss of approximately 1-3% between about 34±20° C. (e.g., 34±10° C. (e.g., 34±5° C. (e.g., 34±4° C. (e.g., 34±3° C. (e.g., 34±2° C. (e.g., 34±1° C. (e.g., 34±0.5° C.))))))) and about 165±20° C. (e.g., 165±10° C. (e.g., 165±5° C. (e.g., 165±4° C. (e.g., 165±3° C. (e.g., 165±2° C. (e.g., 165±1° C. (e.g., 165±0.5° C.))))))), as measured by TGA.

In some embodiments, Form E of Compound No. 1 shows a weight loss of approximately 1.3% between about 34° C. and about 165° C., as measured by TGA.

### Form F

### X-Ray Powder Diffraction (XRPD) Characterization

In some embodiments, the morphic form is Form F of Compound No. 1, the solvate thereof, the hydrate thereof, or the pharmaceutically acceptable salt thereof.

In some embodiments, the morphic form is Form F of the hydrate of Compound No. 1.

In some embodiments, the morphic form is Form F of Compound No. 1.

In some embodiments, the present disclosure provides a form of Compound No. 1 (“Form F”) characterized by an X-ray diffraction (“XRPD”) pattern comprising peaks at 5.5±0.2, 16.4±0.2, and 21.9±0.2°2θ (e.g., 5.5±0.1, 16.4±0.1, and 21.9±0.1°2θ (e.g., 5.5, 16.4, and 21.9°2θ)) using Cu Kα radiation.

In some embodiments, the XRPD pattern of Form F further comprises at least one peak selected from 7.2±0.2, 14.5±0.2, and 15.2±0.2°2θ (e.g., 7.2±0.1, 14.5±0.1, and 15.2±0.1°2θ (e.g., 7.2, 14.5, and 15.2°2θ)) using Cu Kα radiation.

In some embodiments, the XRPD pattern of Form F further comprises at least two peaks selected from 7.2±0.2, 14.5±0.2, and 15.2±0.2°2θ (e.g., 7.2±0.1, 14.5±0.1, and 15.2±0.1°2θ (e.g., 7.2, 14.5, and 15.2°2θ)) using Cu Kα radiation.

In some embodiments, the XRPD pattern of Form F further comprises peaks at 7.2±0.2, 14.5±0.2, and 15.2±0.2°2θ (e.g., 7.2±0.1, 14.5±0.1, and 15.2±0.1°2θ (e.g., 7.2, 14.5, and 15.2°2θ)) using Cu Kα radiation.

In some aspects, the present disclosure provides a form of Compound No. 1 (“Form F”) characterized by an X-ray diffraction (“XRPD”) pattern comprising at least three peaks selected from 5.5±0.2, 7.2±0.2, 14.5±0.2, 15.2±0.2, 16.4±0.2, and 21.9±0.2°2θ (e.g., 5.5±0.1, 7.2±0.1, 14.5±0.1, 15.2±0.1, 16.4±0.1, and 21.9±0.1°2θ (e.g., 5.5, 7.2, 14.5, 15.2, 16.4, and 21.9°2θ)) using Cu Kα radiation.

In some embodiments, the XRPD pattern of Form F comprises at least four peaks selected from 5.5±0.2, 7.2±0.2, 14.5±0.2, 15.2±0.2, 16.4±0.2, and 21.9±0.2°2θ (e.g., 5.5±0.1, 7.2±0.1, 14.5±0.1, 15.2±0.1, 16.4±0.1, and 21.9±0.1°2θ (e.g., 5.5, 7.2, 14.5, 15.2, 16.4, and 21.9°2θ)) using Cu Kα radiation.

In some embodiments, the XRPD pattern of Form F comprises at least five peaks selected from 5.5±0.2, 7.2±0.2, 14.5±0.2, 15.2±0.2, 16.4±0.2, and 21.9±0.2°2θ (e.g., 5.5±0.1, 7.2±0.1, 14.5±0.1, 15.2±0.1, 16.4±0.1, and 21.9±0.1°2θ (e.g., 5.5, 7.2, 14.5, 15.2, 16.4, and 21.9°2θ)) using Cu Kα radiation.

In some embodiments, the XRPD pattern of Form F comprises peaks at 5.5±0.2, 7.2±0.2, 14.5±0.2, 15.2±0.2, 16.4±0.2, and 21.9±0.2°2θ (e.g., 5.5±0.1, 7.2±0.1, 14.5±0.1, 15.2±0.1, 16.4±0.1, and 21.9±0.1°2θ (e.g., 5.5, 7.2, 14.5, 15.2, 16.4, and 21.9°2θ)) using Cu Kα radiation.

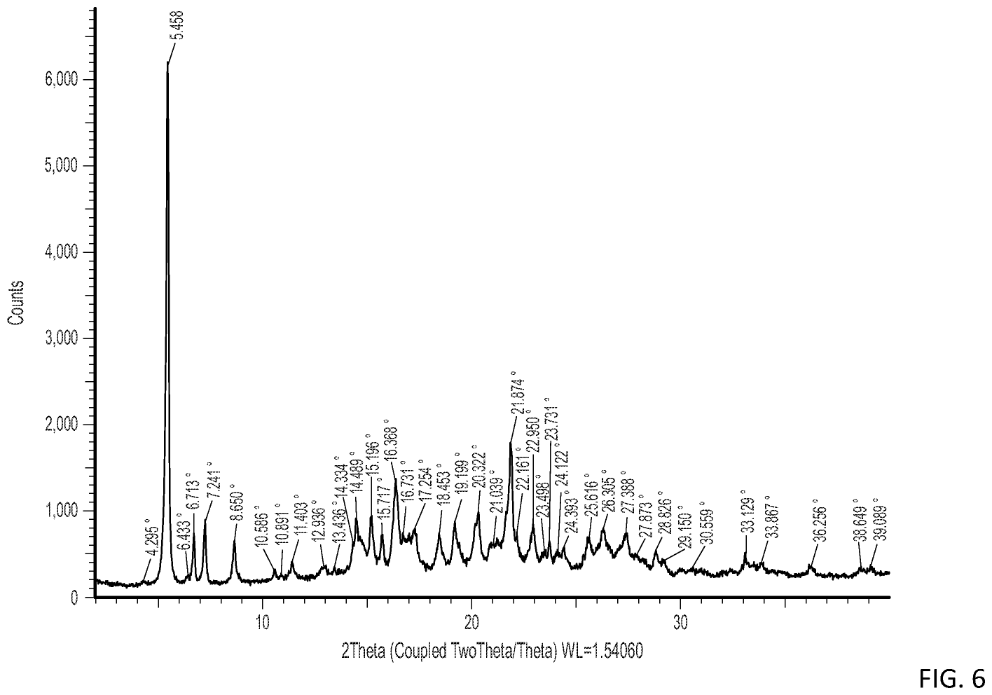

In some embodiments, Form F is characterized by an XRPD pattern substantially similar to that shown in FIG. 6.

In some embodiments, Form F is characterized by an XRPD pattern comprising peaks at approximately the positions shown in the table below. It is understood that the values in the table are approximate and subject to instrumental and experimental variations.

TABLE 6
XRPD peak list for Form F

| | | | Net | Gross | Rel. |
|---|---|---|---|---|---|
| Angle | d Value | | Intensity | Intensity | Intensity |
| 4.295° | 20.55794 | Å | 40.1211 | 181.791 | 0.7% |
| 5.458° | 16.18000 | Å | 5998.00 | 6162.82 | 100.0% |
| 6.433° | 13.72894 | Å | 88.3538 | 238.100 | 3.19% |
| 8.773° | 13.15731 | Å | 549.042 | 718.772 | 9.2% |
| 7.241° | 12.19829 | Å | 690.518 | 856.854 | 11.5% |
| 8.650° | 10.21383 | Å | 475.547 | 845.459 | 7.9% |
| 10.586° | 8.35001 | Å | 92.2115 | 288.902 | 1.5% |
| 10.891° | 8.11685 | Å | 59.4118 | 262.888 | 1.0% |
| 11.403° | 7.75351 | Å | 194.422 | 408.002 | 2.2% |
| 12.938° | 6.83825 | Å | 94.7915 | 342.217 | 3.6% |
| 13.438° | 6.58497 | Å | 75.7246 | 344.086 | 1.3% |
| 14.334° | 6.17423 | Å | 313.334 | 615.701 | 5.2% |
| 14.480° | 8.10859 | Å | 593.021 | 900.976 | 9.9% |
| 15.196° | 5.82598 | Å | 598.891 | 927.555 | 10.0% |
| 15.717° | 5.63398 | Å | 358.824 | 697.708 | 6.0% |
| 16.368° | 5.41108 | Å | 992.445 | 1338.10 | 16.5% |
| 18.731° | 5.29474 | Å | 362.418 | 708.934 | 6.0% |
| 17.254° | 5.13519 | Å | 432.549 | 776.688 | 7.2% |
| 18.453° | 4.80434 | Å | 375.913 | 730.094 | 6.3% |
| 19.199° | 4.51915 | Å | 483.959 | 870.248 | 8.1% |
| 20.322° | 4.36633 | Å | 560.880 | 978.923 | 9.4% |
| 21.039° | 4.21926 | Å | 182.979 | 610.892 | 3.1% |
| 21.039° | 4.21928 | Å | 182.979 | 810.892 | 3.1% |
| 21.874° | 4.06000 | Å | 1354.61 | 1783.84 | 22.6% |
| 22.161° | 4.00809 | Å | 314.617 | 741.756 | 5.2% |
| 22.950° | 3.87209 | Å | 401.259 | 815.988 | 8.7% |
| 23.498° | 3.78297 | Å | 97.1348 | 497.463 | 1.6% |
| 23.731° | 3.74637 | Å | 250.917 | 843.698 | 4.2% |
| 24.122° | 3.68844 | Å | 108.573 | 488.732 | 1.8% |
| 24.393° | 3.64610 | Å | 195.370 | 562.001 | 3.3% |
| 25.616° | 3.47473 | Å | 300.281 | 849.638 | 5.0% |
| 28.305° | 3.38532 | Å | 543.078 | 795.351 | 7.4% |
| 27.388° | 3.25388 | Å | 381.573 | 723.328 | 8.4% |
| 27.873° | 3.19834 | Å | 139.868 | 470.721 | 2.3% |
| 28.826° | 3.09473 | Å | 242.246 | 541.486 | 4.0% |
| 29.150° | 3.06103. | Å | 126.854 | 412.000 | 2.1% |
| 30.559° | 2.92299 | Å | 71.0635 | 331.967 | 1.2% |
| 33.129° | 2.70191 | Å | 165.982 | 437.941 | 2.8% |
| 33.867° | 2.64474 | Å | 118.374 | 387.747 | 1.9% |
| 36.255° | 2.47572 | Å | 103.703 | 353.415 | 3.7% |
| 38.649° | 2.32775 | Å | 92.3595 | 344.570 | 1.5% |
| 39.089° | 2.30259 | Å | 88.1888 | 349.817 | 1.5% |

### Differential Scanning Calorimeter (DSC) Characterizations

In some embodiments, Form F of Compound No. 1 is characterized by a DSC curve having at least one endothermic peak selected from 48±20, 80±20, 146±20, and 174±20° C. (e.g., 48±10, 80±10, 146±10, and 174±10° C. (e.g., 4±8 5, 80±5, 146±5, and 174±5° C. (e.g., 48±4, 80±4, 146±4, and 174±4° C. (e.g., 48±3, 80±3, 146±3, and 174±3° C. (e.g., 48±2,80±2, 146±2, and 174±2° C. (e.g., 48±1,80±1, 146±1, and 174±1° C. (e.g., 48±0.5, 80±0.5, 146±0.5, and 174±0.5° C.))))))).

In some embodiments, Form F of Compound No. 1 is characterized by a DSC curve having at least two endothermic peaks selected from 48±20, 80±20, 146±20, and 174±20° C. (e.g., 48±10, 80±10, 146±10, and 174±10° C. (e.g., 48±5, 80±5, 146±5, and 174±5° C. (e.g., 48±4, 80±4, 146±4, and 174±4° C. (e.g., 48±3, 80±3, 146±3, and 174±3° C. (e.g., 48±2,80±2, 146±2, and 174±2° C. (e.g., 48±1,80±1, 146±1, and 174±1° C. (e.g., 48±0.5, 80±0.5, 146±0.5, and 174±0.5° C.))))))).

In some embodiments, Form F of Compound No. 1 is characterized by a DSC curve having at least three endothermic peaks selected from 48±20, 80±20, 146±20, and 174±20° C. (e.g., 48±10, 80±10, 146±10, and 174±10° C. (e.g., 48±5, 80±5, 146±5, and 174±5° C. (e.g., 48±4, 80±4, 146±4, and 174±4° C. (e.g., 48±3, 80±3, 146±3, and 174±3° C. (e.g., 48±2,80 2, 146±2, and 174±2° C. (e.g., 48±1,80±1, 146±1, and 174±1° C. (e.g., 48 0.5, 80±0.5, 146±0.5, and 174±0.5° C.))))))).

In some embodiments, Form F of Compound No. 1 is characterized by a DSC curve having endothermic peaks at 48±20, 80±20, 146±20, and 174±20° C. (e.g., 48±10, 80±10, 146±10, and 174±10° C. (e.g., 48±5, 80±5, 146±5, and 174±5° C. (e.g., 48±4, 80±4, 146±4, and 174±4° C. (e.g., 48±3, 80±3, 146±3, and 174±3° C. (e.g., 48±2, 80±2, 146±2, and 174±2° C. (e.g., 48±1, 80±1, 146±1, and 174±1° C. (e.g., 48±0.5, 80±0.5, 146±0.5, and 174±0.5° C.))))))).

In some embodiments, Form F of Compound No. 1 is characterized by a DSC curve having an endothermic peak at 48° C. In some embodiments, Form F of Compound No. 1 is characterized by a DSC curve having an endothermic peak at 80° C. In some embodiments, Form F of Compound No. 1 is characterized by a DSC curve having an endothermic peak at 144° C. In some embodiments, Form F of Compound No. 1 is characterized by a DSC curve having an endothermic peak at 146° C. In some embodiments, Form F of Compound No. 1 is characterized by a DSC curve having an endothermic peak at 174° C.

### Thermogravimetric Analysis (7GA) Characterizations

In some embodiments, Form F of Compound No. 1 shows a weight loss of approximately 2-5% between about 33±20° C. (e.g., 33±10° C. (e.g., 33±5° C. (e.g., 33±4° C. (e.g., 33±3° C. (e.g., 33±2° C. (e.g., 33±1° C. (e.g., 33±0.5° C.))))))) and about 55±20° C. (e.g., 55±10° C. (e.g., 55±5° C. (e.g., 55±4° C. (e.g., 55±3° C. (e.g., 55±2° C. (e.g., 55±1° C. (e.g., 55±0.5° C.))))))), as measured by TGA.

In some embodiments, Form F of Compound No. 1 shows a weight loss of approximately 3.8% between about 33° C. and about 55° C., as measured by TGA.

In some embodiments, Form F of Compound No. 1 shows a weight loss of approximately 1-4% between about 55±20° C. (e.g., 55±10° C. (e.g., 55±5° C. (e.g., 55±4° C. (e.g., 55±3° C. (e.g., 55±2° C. (e.g., 55±1° C. (e.g., 55±0.5° C.))))))) and about 100±20° C. (e.g., 100±10° C. (e.g., 100±5′ C (e.g., 100±4° C. (e.g., 100±3° C. (e.g., 100±2° C. (e.g., 100±1° C. (e.g., 100 t 0.5° C.))))))), as measured by TGA.

In some embodiments, Form F of Compound No. 1 shows a weight loss of approximately 2.7% between about 55° C. and about 100° C., as measured by TGA.

In some embodiments, Form F of Compound No. 1 shows a weight loss of approximately 2-8% between about 33±20° C. (e.g., 33±10° C. (e.g., 33±5′ C (e.g., 33±4° C. (e.g., 33±3° C. (e.g., 33±2° C. (e.g., 33±1° C. (e.g., 33±0.5° C.))))))) and about 100±20° C. (e.g., 100±10° C. (e.g., 100±5° C. (e.g., 100±4° C. (e.g., 100±3° C. (e.g., 100±2° C. (e.g., 100±1° C. (e.g., 100±0.5° C.))))))), as measured by TGA.

In some embodiments, Form F of Compound No. 1 shows a weight loss of approximately 6.5% between about 33° C. and about 100° C., as measured by TGA.

In some embodiments, Form F of Compound No. 1 shows a weight loss of approximately 3.7% between about 33° C. and about 100° C., as measured by TGA.

### Form G

### X-Ray Powder Diffraction (WRPD) Characterization

In some embodiments, the morphic form is Form G of Compound No. 1, the solvate thereof, the hydrate thereof, or the pharmaceutically acceptable salt thereof.

In some embodiments, the morphic form is Form G of Compound No. 1.

In some embodiments, the present disclosure provides a form of Compound No. 1 (“Form G”) characterized by an X-ray diffraction (“XRPD”) pattern comprising peaks at 4.0±0.2, 5.3±0.2, and 16.0±0.2°2θ (e.g., 4.0±0.1, 5.3±0.1, and 16.0±0.1°2θ (e.g., 4.0, 5.3, and 16.0°2θ)) using Cu Kα radiation.

In some embodiments, the XRPD pattern of Form G further comprises at least one peak selected from 7.1±0.2, 16.7±0.2, and 19.2±0.2°2θ (e.g., 7.1±0.1, 16.7±0.1, and 19.2±0.1°2θ (e.g., 7.1, 16.7, and 19.2°2θ)) using Cu Kα radiation.

In some embodiments, the XRPD pattern of Form G further comprises at least two peaks selected from 7.1±0.2, 16.7±0.2, and 19.2±0.2°2θ (e.g., 7.1±0.1, 16.7±0.1, and 19.2±0.1°2θ (e.g., 7.1, 16.7, and 19.2°2θ)) using Cu Kα radiation.

In some embodiments, the XRPD pattern of Form G further comprises peaks at 7.1±0.2, 16.7±0.2, and 19.2±0.2°2θ (e.g., 7.1±0.1, 16.7±0.1, and 19.2±0.1°2θ (e.g., 7.1, 16.7, and 19.2°2θ)) using Cu Kα radiation.

In some aspects, the present disclosure provides a form of Compound No. 1 (“Form G”) characterized by an X-ray diffraction (“XRPD”) pattern comprising at least three peaks selected from 4.0±0.2, 5.3±0.2, 7.1±0.2, 16.0±0.2, 16.7±0.2, and 19.2±0.2°2θ (e.g., 4.0±0.1, 5.3±0.1, 7.1±0.1, 16.0±0.1, 16.7±0.1, and 19.2±0.1°2θ (e.g., 4.0, 5.3, 7.1, 16.0, 16.7, and 19.2°2θ)) using Cu Kα radiation.

In some embodiments, the XRPD pattern of Form G comprises at least four peaks selected from 4.0±0.2, 5.3±0.2, 7.1±0.2, 16.0±0.2, 16.7±0.2, and 19.2±0.2°2θ (e.g., 4.0±0.1, 5.3±0.1, 7.1±0.1, 16.0±0.1, 16.7±0.1, and 19.2±0.1°2θ (e.g., 4.0, 5.3, 7.1, 16.0, 16.7, and 19.2°2θ)) using Cu Kα radiation.

In some embodiments, the XRPD pattern of Form G comprises at least five peaks selected from 4.0±0.2, 5.3±0.2, 7.1±0.2, 16.0±0.2, 16.7±0.2, and 19.2±0.2°2θ (e.g., 4.0±0.1, 5.3±0.1, 7.1±0.1, 16.0±0.1, 16.7±0.1, and 19.2±0.1°2θ (e.g., 4.0, 5.3, 7.1, 16.0, 16.7, and 19.2°2θ)) using Cu Kα radiation.

In some embodiments, the XRPD pattern of Form G comprises peaks at 4.0±0.2, 5.3±0.2, 7.1±0.2, 16.0±0.2, 16.7±0.2, and 19.2±0.2°2θ (e.g., 4.0±0.1, 5.3±0.1, 7.1±0.1, 16.0±0.1, 16.7±0.1, and 19.2±0.1°2θ (e.g., 4.0, 5.3, 7.1, 16.0, 16.7, and 19.2°2θ)) using Cu Kα radiation.

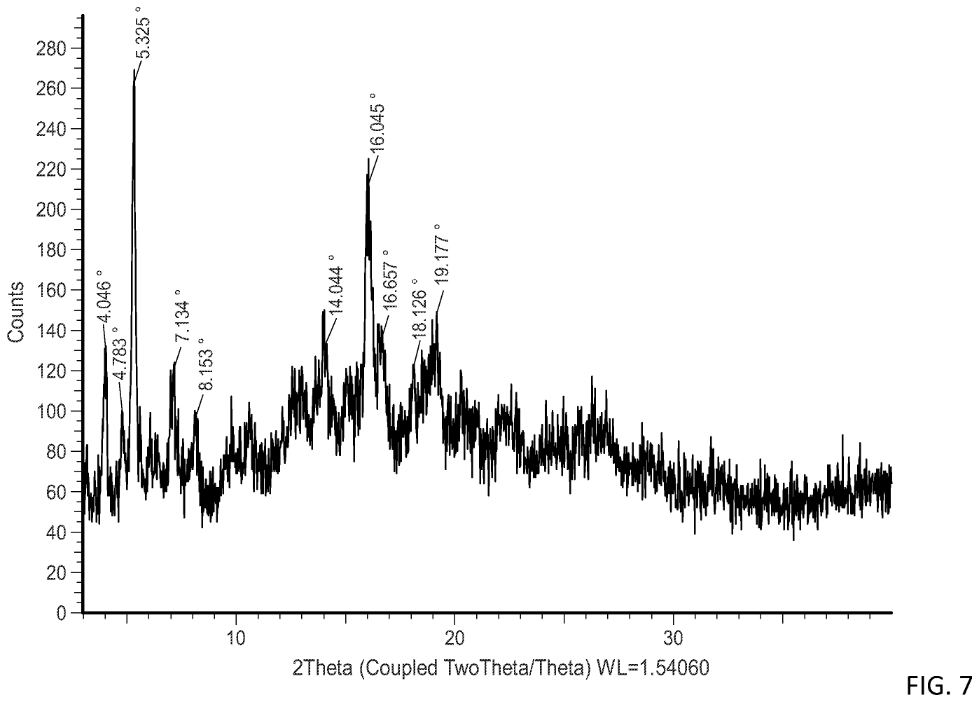

In some embodiments, Form G is characterized by an XRPD pattern substantially similar to that shown in FIG. 7.

In some embodiments, Form G is characterized by an XRPD pattern comprising peaks at approximately the positions shown in the table below. It is understood that the values in the table are approximate and subject to instrumental and experimental variations.

TABLE 7
XRPD peak list for Form G

| | | | Net | Gross | Rel |
|---|---|---|---|---|---|
| Angle | d Value | | Intensity | Intensity | Intensity |
| 4.046° | 21.82289 | Å | 73.3860 | 128.843 | 36.3% |
| 4.783° | 18.46200 | Å | 42.0186 | 99.7441 | 20.8% |
| 5.325° | 16.58167 | Å | 202.262 | 262.643 | 100.0% |
| 7.134° | 12.38174 | Å | 58.1103 | 120.371 | 28.7% |
| 8.153° | 10.83555 | Å | 38.1599 | 86.7500 | 18.9% |
| 14.044° | 6.30089 | Å | 46.4580 | 132.102 | 23.0% |
| 16.045° | 5.51932 | Å | 125.382 | 212.028 | 62.0% |
| 18.857° | 5.31804 | Å | 51.3215 | 135.982 | 25.4% |
| 18.126° | 4.88028 | Å | 37.3803 | 122.388 | 18.5% |
| 19.177° | 4.62457 | Å | 61.1202 | 148.378 | 30.2% |

### Differential Scanning Calorimeter (DSC) Characterizations

In some embodiments, Form G of Compound No. 1 is characterized by a DSC curve having at least one endothermic peak selected from 34±20, 175±20, and 182±20° C. (e.g., 34±10, 175±10, and 182±10° C. (e.g., 34±5, 175±5, and 182±5° C. (e.g., 34±4, 175±4, and 182±4° C. (e.g., 34±3, 175±3, and 182±3° C. (e.g., 34±2, 175±2, and 182±2° C. (e.g., 34±1, 175±1, and 182±1° C. (e.g., 34±0.5, 175±0.5, and 182±0.5° C.))))))).

In some embodiments, Form G of Compound No. 1 is characterized by a DSC curve having at least two endothermic peaks selected from 34±20, 175±20, and 182±20° C. (e.g., 34±10, 175±10, and 182±10° C. (e.g., 34±5, 175±5, and 182±5° C. (e.g., 34±4, 175±4, and 182±4° C. (e.g., 34±3, 175±3, and 182±3° C. (e.g., 34±2, 175±2, and 182±2° C. (e.g., 34±1, 175±1, and 182±1° C. (e.g., 34±0.5, 175±0.5, and 182±0.5° C.))))))).

In some embodiments, Form G of Compound No. 1 is characterized by a DSC curve having endothermic peaks at 34±20, 175±20, and 182±20° C. (e.g., 34±10, 175±10, and 182±10° C. (e.g., 34±5, 175±5, and 182±5° OC (e.g., 34±4, 175±4, and 182±4° C. (e.g., 34±3, 175±3, and 182±3° C. (e.g., 34±2, 175±2, and 182±2° C. (e.g., 34±1, 175±1, and 182±1° C. (e.g., 34±0.5, 175±0.5, and 182±0.5° C.))))))).

In some embodiments, Form G of Compound No. 1 is characterized by a DSC curve having an endothermic peak at 34° C. In some embodiments, Form G of Compound No. 1 is characterized by a DSC curve having an endothermic peak at 175° C. In some embodiments, Form G of Compound No. 1 is characterized by a DSC curve having an endothermic peak at 182° C.

### Thermogravimetric Analysis (7GA) Characterizations

In some embodiments, Form G of Compound No. 1 shows a weight loss of approximately 2-5% between about 33±20° C. (e.g., 33±10° C. (e.g., 33±5° C. (e.g., 33±4° C. (e.g., 33±3° C. (e.g., 33±2° C. (e.g., 33±1° C. (e.g., 33±0.5° C.))))))) and about 100±20° C. (e.g., 100±10° C. (e.g., 100±5° C. (e.g., 100±4° C. (e.g., 100±3° C. (e.g., 100±2° C. (e.g., 100±1° C. (e.g., 100±0.5° C.))))))), as measured by TGA.

In some embodiments, Form G of Compound No. 1 shows a weight loss of approximately 3.4% between about 33° C. and about 100° C., as measured by TGA.

### Form H

### X-Ray Powder Diffraction (XRPD) Characterization

In some embodiments, the morphic form is Form H of Compound No. 1, the solvate thereof, the hydrate thereof, or the pharmaceutically acceptable salt thereof.

In some embodiments, the morphic form is Form H of Compound No. 1.

In some embodiments, the present disclosure provides a form of Compound No. 1 (“Form H”) characterized by an X-ray diffraction (“XRPD”) pattern comprising peaks at 4.6±0.2, 13.8±0.2, and 17.0±0.2°2θ (e.g., 4.6±0.1, 13.8±0.1, and 17.0±0.1°2θ (e.g., 4.6, 13.8, and 17.0°2θ)) using Cu Kα radiation.

In some embodiments, the XRPD pattern of Form H further comprises at least one peak selected from 5.6±0.2, 8.6±0.2, and 17.8±0.2°2θ (e.g., 5.6±0.1, 8.6±0.1, and 17.8±0.1°2θ (e.g., 5.6, 8.6, and 17.8°2θ)) using Cu Kα radiation.

In some embodiments, the XRPD pattern of Form H further comprises at least two peaks selected from 5.6±0.2, 8.6±0.2, and 17.8±0.2°2θ (e.g., 5.6±0.1, 8.6±0.1, and 17.8±0.1°2θ (e.g., 5.6, 8.6, and 17.8°2θ)) using Cu Kα radiation.

In some embodiments, the XRPD pattern of Form H further comprises peaks at 5.6±0.2, 8.6±0.2, and 17.8±0.2°2θ (e.g., 5.6±0.1, 8.6±0.1, and 17.8±0.1°2θ (e.g., 5.6, 8.6, and 17.8°2θ)) using Cu Kα radiation.

In some aspects, the present disclosure provides a form of Compound No. 1 (“Form H”) characterized by an X-ray diffraction (“XRPD”) pattern comprising at least three peaks selected from 4.6±0.2, 5.6±0.2, 8.6±0.2, 13.8±0.2, 17.0±0.2, and 17.8±0.2°2θ (e.g., 4.6±0.1, 5.6±0.1, 8.6±0.1, 13.8±0.1, 17.0±0.1, and 17.8±0.1°2θ (e.g., 4.6, 5.6, 8.6, 13.8, 17.0, and 17.8°2θ)) using Cu Kα radiation.

In some embodiments, the XRPD pattern of Form H comprises at least four peaks selected from 4.6±0.2, 5.6±0.2, 8.6±0.2, 13.8±0.2, 17.0±0.2, and 17.8±0.2°2θ (e.g., 4.6±0.1, 5.6±0.1, 8.6±0.1, 13.8±0.1, 17.0±0.1, and 17.8±0.1°2θ (e.g., 4.6, 5.6, 8.6, 13.8, 17.0, and 17.8°2θ)) using Cu Kα radiation.

In some embodiments, the XRPD pattern of Form H comprises at least five peaks selected from 4.6±0.2, 5.6±0.2, 8.6±0.2, 13.8±0.2, 17.0±0.2, and 17.8±0.2°2θ (e.g., 4.6±0.1, 5.6±0.1, 8.6±0.1, 13.8±0.1, 17.0±0.1, and 17.8±0.1°2θ (e.g., 4.6, 5.6, 8.6, 13.8, 17.0, and 17.8°2θ)) using Cu Kα radiation.

In some embodiments, the XRPD pattern of Form H comprises peaks at 4.6±0.2, 5.6±0.2, 8.6±0.2, 13.8±0.2, 17.0±0.2, and 17.8±0.2°2θ (e.g., 4.6±0.1, 5.6±0.1, 8.6±0.1, 13.8±0.1, 17.0±0.1, and 17.8±0.1°2θ (e.g., 4.6, 5.6, 8.6, 13.8, 17.0, and 17.8°2θ)) using Cu Kα radiation.

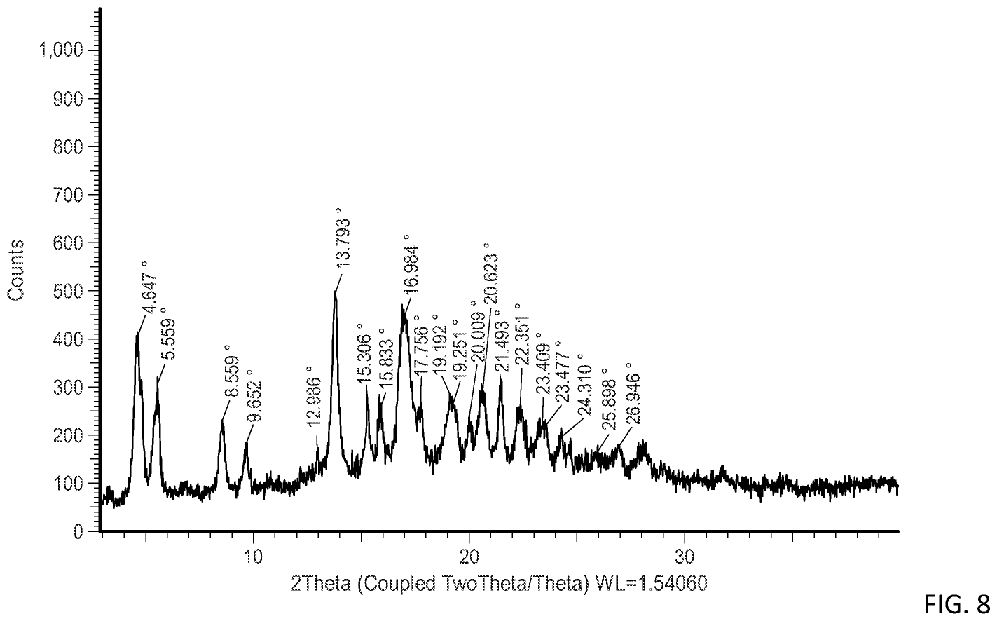

In some embodiments, the present disclosure provides a form of Compound No. 1 (“Form H”), characterized by an XRPD pattern substantially similar to that shown in FIG. 8.

In some embodiments, Form H is characterized by an XRPD pattern comprising peaks at approximately the positions shown in the table below. It is understood that the values in the table are approximate and subject to instrumental and experimental variations.

TABLE 8
XRPD peak list for Form H

| | | | Net | Gross | Rel. | | |
|---|---|---|---|---|---|---|---|
| | | | Inten- | Inten- | Inten- | C. | h, k, |
| Angle | d Value | | sity | sity | sity | Size | l |
| 4. [symbol] 47° | [symbol] | Å | [symbol] | 406.721 | 90.0% | z | n.a. |
| 5.559° | [symbol] | Å | [symbol] | [symbol] | [symbol] | | n.a. |
| 8.55 [symbol] ° | 10.32237 | Å | 147.235 | [symbol] | [symbol] | | n.a. |
| [symbol] 52° | [symbol] | Å | 90.7722 | [symbol] | 28.1% | | n.a. |
| 12 [symbol] ° | [symbol] | Å | [symbol] | [symbol] | 11.4% | | n.a. |
| 1 [symbol] ° | [symbol] | Å | [symbol] | 492.558 | 100.0% | | n.a. |
| 15. [symbol] 06° | 5.78417 | Å | [symbol] | 255.456 | [symbol] | | n.a. |
| 15. [symbol] ° | 5.59283 | Å | 111.741 | 247.009 | 29.7% | | n.a. |
| 1 [symbol] 4° | [symbol] | Å | [symbol] | 443.280 | [symbol] | | n.a. |
| 17.756° | 4.99115 | Å | [symbol] | [symbol] | 34.7% | | n.a. |
| 1 [symbol] .192° | [symbol] | Å | 100.622 | [symbol] | 26.7% | | n.a. |
| 1 [symbol] .251° | [symbol] | Å | [symbol] | 271.005 | 31.2% | | n.a. |
| 20.00 [symbol] ° | [symbol] | Å | [symbol] | [symbol] | 21.7% | | n.a. |
| 20. [symbol] 23° | 4.30337 | Å | [symbol] | [symbol] | 32.2% | | n.a. |
| 21. [symbol] ° | 4 13106 | Å | [symbol] | [symbol] | 34.1% | | n.a. |
| 22. [symbol] 51° | 3.97447 | Å | 92.1414 | [symbol] | 24.5% | | n.a. |
| 23.409° | 3.79713 | Å | 73.6040 | [symbol] | [symbol] | | n.a. |
| 2 [symbol] .477° | [symbol] | Å | [symbol] | [symbol] | [symbol] | | n.a. |
| 24.311° | [symbol] | Å | [symbol] | [symbol] | [symbol] | | n.a. |
| 2 [symbol] ° | 3.43754 | Å | 31.5270 | 161.202 | [symbol] | | n.a. |
| 2 [symbol] ° | 3.30617 | Å | [symbol] | [symbol] | [symbol] | | n.a. |

[symbol]

indicates data missing or illegible when filed

### Differential Scanning Calorimeter (DSC) Characterizations

In some aspects, the present disclosure provides a form of Compound No. 1 (“Form H”), characterized by a DSC curve having at least one endothermic peak selected from 62±20 and 153±20′C (e.g., 62±10 and 153±10′C (e.g., 62±5 and 153±5° C. (e.g., 62±4 and 153±4° C. (e.g., 62±3 and 153±3° C. (e.g., 62±2 and 153±2° C. (e.g., 62±1 and 153±1° C. (e.g., 62±0.5 and 153±0.5° C.))))))).

In some embodiments, Form H of Compound No. 1 is characterized by a DSC curve having endothermic peaks at 62±20 and 153±20° C. (e.g., 62±10 and 153±10° C. (e.g., 62±5 and 153±5° C. (e.g., 62±4 and 153±4° C. (e.g., 62±3 and 153±3° C. (e.g., 62±2 and 153±2° C. (e.g., 62±1 and 153±1° C. (e.g., 62±0.5 and 153±0.5° C.))))))).

In some embodiments, Form H of Compound No. 1 is characterized by a DSC curve having an endothermic peak at 62° C. In some embodiments, Form H of Compound No. 1 is characterized by a DSC curve having an endothermic peak at 153° C.

### Form I

### X-Ray Powder Diffraction (XRPD) Characterization

In some embodiments, the morphic form is Form I of Compound No. 1, the solvate thereof, the hydrate thereof, or the pharmaceutically acceptable salt thereof.

In some embodiments, the morphic form is Form I of the methanol solvate of Compound No. 1.

In some embodiments, the morphic form is Form I of Compound No. 1.

In some embodiments, the present disclosure provides a form of Compound No. 1 (“Form I”) characterized by an X-ray diffraction (“XRPD”) pattern comprising peaks at 5.1±0.2, 20.4±0.2, and 21.5±0.2°2θ (e.g., 5.1±0.1, 20.4±0.1, and 21.5±0.1°2θ (e.g., 5.1, 20.4, and 21.5°2θ)) using Cu Kα radiation.

In some embodiments, the XRPD pattern of Form I further comprises at least one peak selected from 17.0±0.2, 22.3±0.2, and 25.5±0.2°2θ (e.g., 17.0±0.1, 22.3±0.1, and 25.5±0.1°2θ (e.g., 17.0, 22.3, and 25.5°2θ)) using Cu Kα radiation.

In some embodiments, the XRPD pattern of Form I further comprises at least two peaks selected from 17.0±0.2, 22.3±0.2, and 25.5±0.2°2θ (e.g., 17.0±0.1, 22.3±0.1, and 25.5±0.1°2θ (e.g., 17.0, 22.3, and 25.5°2θ)) using Cu Kα radiation.

In some embodiments, the XRPD pattern of Form I further comprises peaks at 17.0±0.2, 22.3±0.2, and 25.5±0.2°2θ (e.g., 17.0±0.1, 22.3±0.1, and 25.5±0.1°2θ (e.g., 17.0, 22.3, and 25.5°2θ)) using Cu Kα radiation.

In some aspects, the present disclosure provides a form of Compound No. 1 (“Form I”) characterized by an X-ray diffraction (“XRPD”) pattern comprising at least three peaks selected from 5.1±0.2, 17.0±0.2, 20.4±0.2, 21.5±0.2, 22.3±0.2, and 25.5±0.2°2θ (e.g., 5.1±0.1, 17.0±0.1, 20.4±0.1, 21.5±0.1, 22.3±0.1, and 25.5±0.1°2θ (e.g., 5.1, 17.0, 20.4, 21.5, 22.3, and 25.5°2θ)) using Cu Kα radiation.

In some embodiments, the XRPD pattern of Form I comprises at least four peaks selected from 5.1±0.2, 17.0±0.2, 20.4±0.2, 21.5±0.2, 22.3±0.2, and 25.5±0.2°2θ (e.g., 5.1±0.1, 17.0±0.1, 20.4±0.1, 21.5±0.1, 22.3±0.1, and 25.5±0.1°2θ (e.g., 5.1, 17.0, 20.4, 21.5, 22.3, and 25.5°2θ)) using Cu Kα radiation.

In some embodiments, the XRPD pattern of Form I comprises at least five peaks selected from 5.1±0.2, 17.0±0.2, 20.4±0.2, 21.5±0.2, 22.3±0.2, and 25.5±0.2°2θ (e.g., 5.1±0.1, 17.0±0.1, 20.4±0.1, 21.5±0.1, 22.3±0.1, and 25.5±0.1°2θ (e.g., 5.1, 17.0, 20.4, 21.5, 22.3, and 25.5°2θ)) using Cu Kα radiation.

In some embodiments, the XRPD pattern of Form I comprises peaks at 5.1±0.2, 17.0±0.2, 20.4±0.2, 21.5±0.2, 22.3±0.2, and 25.5±0.2±90 (e.g., 5.1±0.1, 17.0±0.1, 20.4±0.1, 21.5±0.1 22.3±0.1, and 25.5±0.1°2θ (e.g., 5.1, 17.0, 20.4±21.5, 22.3, and 25.5±28)) using Cu Kα radiation.

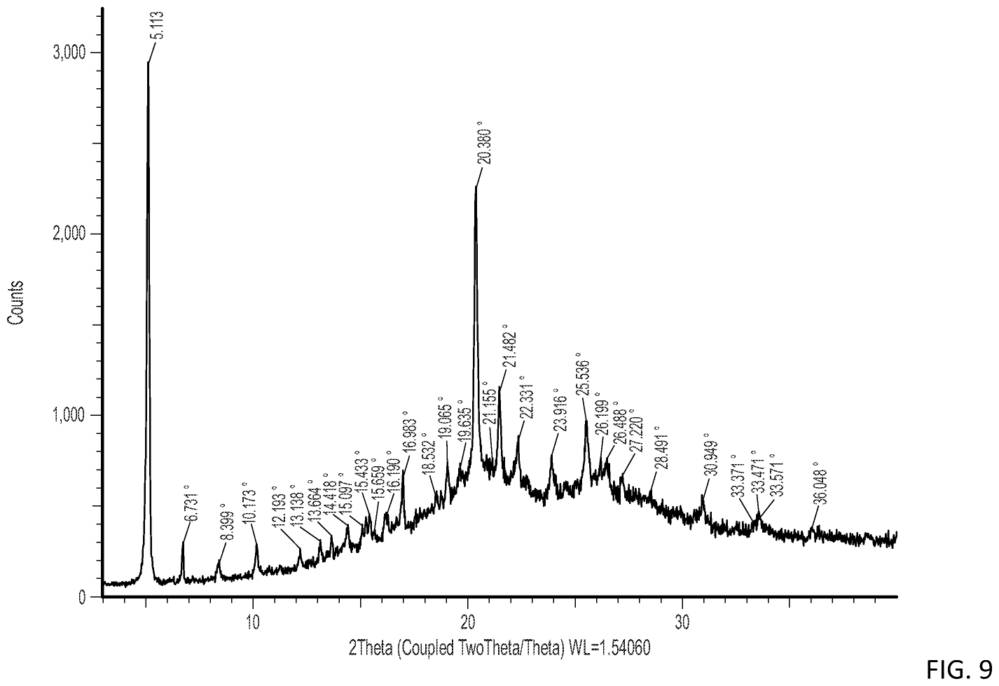

In some embodiments, Form I is characterized by an XRPD pattern substantially similar to that shown in FIG. 9.

In some embodiments, Form I is characterized by an XRPD pattern comprising peaks at approximately the positions shown in the table below. It is understood that the values in the table are approximate and subject to instrumental and experimental variations.

TABLE 9
XRPD peak list for Form I

| | | | Net | Gross | Rel. |
|---|---|---|---|---|---|
| Angle | d Value | | Intensity | Intensity | Intensity |
| 5.113° | 17.26933 | Å | 2853.47 | 2931.22 | 100.0% |
| 6.731° | 13.12189 | Å | [symbol] | 286.654 | 7.3% |
| 8.399° | 10.51907 | Å | 76.8335 | 173.138 | 2.7% |
| 10.173° | [symbol] | Å | 165.591 | 287.461 | 5.8% |
| [symbol] | 7.25286 | Å | 102.595 | 263.625 | 3.6% |
| 13.138° | 6.73326 | Å | 109.995 | 304.421 | 3.9% |
| 13.664° | 6.47550 | Å | 115.901 | 334.917 | 4.1% |
| [symbol] | [symbol] | Å | 141.875 | 391.800 | 5.0% |
| 15.097° | [symbol] | Å | 113.728 | 391.091 | 4.0% |
| 15.433° | [symbol] | Å | 153.463 | [symbol] | 5.4% |
| [symbol] | 5.65458 | Å | 52.5765 | 359.184 | 1.8% |
| 16.190° | 5.47019 | Å | 101.013 | 435.305 | 3.5% |
| [symbol] | 5.21652 | Å | 277.053 | 653.770 | 9.7% |
| 18.532° | 4.78390 | Å | 91.3707 | 547.100 | 3.2% |
| 19.065° | 4.65142 | Å | 246.944 | 726.434 | 8.7% |
| 19.635° | 4.51755 | Å | [symbol] | 697.201 | 6.9% |
| 20.380° | 4.35418 | Å | 1717.11 | 2242.06 | 60.2% |
| 21.155° | 4.19637 | Å | 211.002 | 753.963 | 7.4% |
| [symbol] | 4.13310 | Å | [symbol] | 1126.87 | 20.3% |
| 22.331° | 3.97784 | Å | 289.223 | [symbol] | 10.1% |
| 23.916° | 3.71783 | Å | 214.347 | 778.177 | 7.5% |
| 25.536° | 3.48546 | Å | 401.114 | [symbol] | 14.1% |
| [symbol] | [symbol] | Å | [symbol] | 756.556 | 7.1% |
| [symbol] | 3.36229 | Å | 207.081 | 757.012 | 7.3% |
| 27.221° | 3.27347 | Å | 135.295 | [symbol] | 4.7% |
| [symbol] | 3.13033 | Å | 76.2228 | [symbol] | 2.7% |
| 30.949° | 2.88707 | Å | 127.393 | 527.416 | 4.5% |
| 33.371° | 2.68284 | Å | 50.8639 | 389.178 | 1.8% |
| 33.471° | 2.67512 | Å | 64.7342 | 411.575 | 2.3% |
| [symbol] | 2.66738 | Å | 119.002 | 464.246 | 4.2% |
| 36.048° | [symbol] | Å | 62.2027 | 378.374 | 2.2% |

[symbol]

indicates data missing or illegible when filed

### Form J

In some embodiments, the morphic form is Form J of Compound No. 1, the solvate thereof, the hydrate thereof, or the pharmaceutically acceptable salt thereof.

In some embodiments, the morphic form is Form J of the hydrate of Compound No. 1.

In some embodiments, the morphic form is Form J of Compound No. 1.

In some embodiments, the present disclosure provides a form of Compound No. 1 (“Form F”) characterized by an X-ray diffraction (“XRPD”) pattern comprising peaks at 4.5±0.2, 17.9±0.2, and 22.5±0.2°2θ (e.g., 4.5±0.1, 17.9±0.1, and 22.5±0.1°2θ (e.g., 4.5, 17.9, and 22.5°2θ)) using Cu Kα radiation.

In some embodiments, the XRPD pattern of Form J further comprises at least one peak selected from 5.1±0.2, 10.4±0.2, and 16.0±0.2°2θ (e.g., 5.1±0.1, 10.4±0.1, and 16.0±0.1°2θ (e.g., 5.1, 10.4, and 16.0°2θ)) using Cu Kα radiation.

In some embodiments, the XRPD pattern of Form J further comprises at least two peaks selected from 5.1±0.2, 10.4±0.2, and 16.0±0.2°2θ (e.g., 5.1±0.1, 10.4±0.1, and 16.0±0.1°2θ (e.g., 5.1, 10.4, and 16.0°2θ)) using Cu Kα radiation.

In some embodiments, the XRPD pattern of Form J further comprises peaks at 5.1±0.2, 10.4±0.2, and 16.0±0.2°2θ (e.g., 5.1±0.1, 10.4±0.1, and 16.0±0.1°2θ (e.g., 5.1, 10.4, and 16.0°2θ)) using Cu Kα radiation.

In some aspects, the present disclosure provides a form of Compound No. 1 (“Form J”) characterized by an X-ray diffraction (“XRPD”) pattern comprising at least three peaks selected from 4.5±0.2, 5.1±0.2, 10.4±0.2, 16.0±0.2, 17.9±0.2, and 22.5±0.2°2θ (e.g., 4.5±0.1, 5.1±0.1, 10.4±0.1, 16.0±0.1, 17.9±0.1, and 22.5±0.1°2θ (e.g., 4.5, 5.1, 10.4, 16.0, 17.9, and 22.5°2θ)) using Cu Kα radiation.

In some embodiments, the XRPD pattern of Form J comprises at least four peaks selected from 4.5±0.2, 5.1±0.2, 10.4±0.2, 16.0±0.2, 17.9±0.2, and 22.5±0.2°2θ (e.g., 4.5±0.1, 5.1±0.1, 10.4±0.1, 16.0±0.1, 17.9±0.1, and 22.5±0.1°2θ (e.g., 4.5, 5.1, 10.4, 16.0, 17.9, and 22.5°2θ)) using Cu Kα radiation.

In some embodiments, the XRPD pattern of Form J comprises at least five peaks selected from 4.5±0.2, 5.1±0.2, 10.4±0.2, 16.0±0.2, 17.9±0.2, and 22.5±0.2°2θ (e.g., 4.5±0.1, 5.1±0.1, 10.4±0.1, 16.0±0.1, 17.9±0.1, and 22.5±0.1°2θ (e.g., 4.5, 5.1, 10.4, 16.0, 17.9, and 22.5°2θ)) using Cu Kα radiation.

In some embodiments, the XRPD pattern of Form J comprises peaks at 4.5±0.2, 5.1±0.2, 10.4±0.2, 16.0±0.2, 17.9±0.2, and 22.5±0.2°2θ (e.g., 4.5±0.1, 5.1±0.1, 10.4±0.1, 16.0±0.1, 17.9±0.1, and 22.5±0.1°2θ (e.g., 4.5, 5.1, 10.4, 16.0, 17.9, and 22.5°2θ)) using Cu Kα radiation.

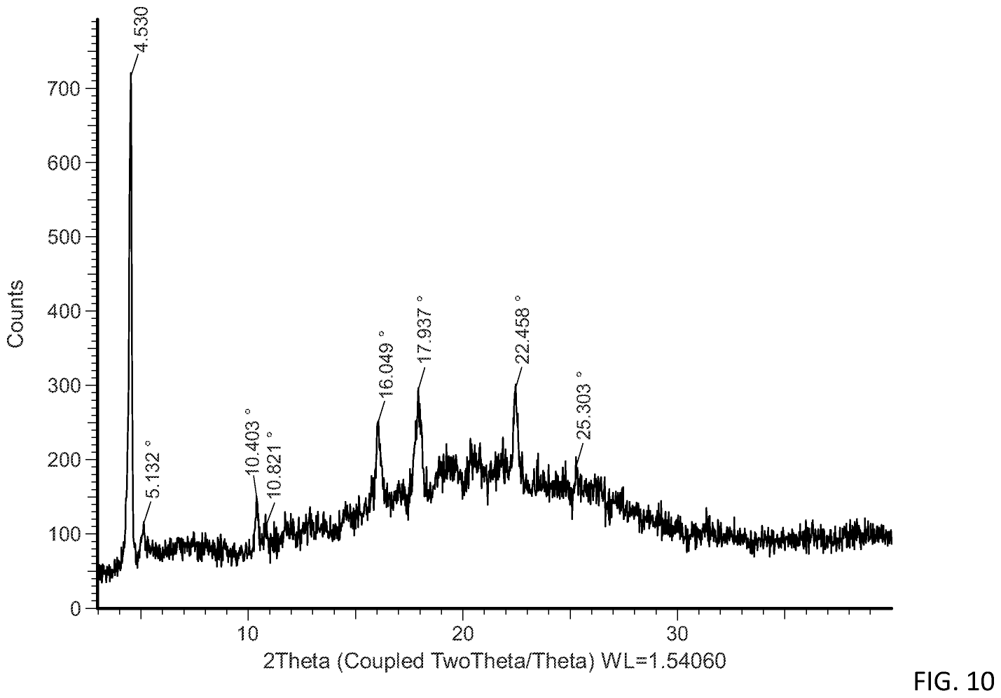

In some embodiments, Form J is characterized by an XRPD pattern substantially similar to that shown in FIG. 10.

In some embodiments, Form J is characterized by an XRPD pattern comprising peaks at approximately the positions shown in the table below. It is understood that the values in the table are approximate and subject to instrumental and experimental variations.

TABLE 10
XRPD peak list for Form J

| | | | Net | Gross | Rel. |
|---|---|---|---|---|---|
| Angle | d Value | | Intensity | Intensity | Intensity |
| 4.530° | 19.49209 | Å | [symbol] | 715.604 | 100.0% |
| 5.132° | 17.20664 | Å | 46.1089 | 113.389 | 7.0% |
| 10.403° | 8.49634 | Å | 71.7435 | 147.200 | 10.9% |
| 10.821° | 8.16815 | Å | 33.2594 | 110.592 | 5.1% |
| 16.049° | 5.51820 | Å | 114.630 | 248.543 | 17.5% |
| 17.937° | 4.94118 | Å | 142.384 | 293.808 | 21.7% |
| 22.458° | 3.95578 | Å | 125.276 | 294.468 | 19.1% |
| 25.303° | 3.51697 | Å | 45.0854 | 191.680 | 6.9% |

[symbol]

indicates data missing or illegible when filed

### Differential Scanning Calorimeter (DSC) Characterizations

In some embodiments, Form J of Compound No. 1 is characterized by a DSC curve having at least one endothermic peak selected from 137±20 and 166±20° C. (e.g., 137±10 and 166±10° C. (e.g., 137±5 and 166±5° C. (e.g., 137±4 and 166±4° C. (e.g., 137±3 and 166±3° C. (e.g., 137±2 and 166±2° C. (e.g., 137±1 and 166±1° C. (e.g., 137±0.5 and 166±0.5° C.))))))).

In some embodiments, Form J of Compound No. 1 is characterized by a DSC curve having endothermic peaks at 137±20 and 166±20° C. (e.g., 137±10 and 166±10° C. (e.g., 137+5 and 166±5° C. (e.g., 137±4 and 166±4° C. (e.g., 137±3 and 166±3° C. (e.g., 137±2 and 166±2° C. (e.g., 137±1 and 166±1° C. (e.g., 137±0.5 and 166±0.5° C.))))))).

In some embodiments, Form J of Compound No. 1 is characterized by a DSC curve having an endothermic peak at 137° C. In some embodiments, Form J of Compound No. 1 is characterized by a DSC curve having an endothermic peak at 166° C.

### Thermogravimetric Analysis (TGA) Characterizations

In some embodiments, Form J of Compound No. 1 shows a weight loss of approximately 1-2% between about 33±20° C. (e.g., 33±10° C. (e.g., 33±5° C. (e.g., 33±4° C. (e.g., 33±3° C. (e.g., 33±2° C. (e.g., 33±1° C. (e.g., 33±0.5° C.))))))) and about 75±20° C. (e.g., 75±10° C. (e.g., 75±5° C. (e.g., 75±4° C. (e.g., 75±3° C. (e.g., 75±2° C. (e.g., 75±1° C. (e.g., 75±0.5° C.))))))), as measured by TGA.

In some embodiments, Form J of Compound No. 1 shows a weight loss of approximately 1.1% between about 33° C. and about 75° C., as measured by TGA.

In some embodiments, Form J of Compound No. 1 shows a weight loss of approximately 6-8% between about 75±20° C. (e.g., 75±10° C. (e.g., 75±5° C. (e.g., 75±4° C. (e.g., 75±3° C. (e.g., 75±2° C. (e.g., 75±1° C. (e.g., 75±0.5° C.))))))) and about 175±20° C. (e.g., 175±10° C. (e.g., 175±5° C. (e.g., 175±4° C. (e.g., 175±3° C. (e.g., 175±2° C. (e.g., 175±1C (e.g., 175±0.5° C.))))))), as measured by TGA.

In some embodiments, Form J of Compound No. 1 shows a weight loss of approximately 7.0% between about 75° C. and about 175° C., as measured by TGA.

In some embodiments, Form J of Compound No. 1 shows a weight loss of approximately 7-10% between about 33±20° C. (e.g., 33±10° C. (e.g., 33±5° C. (e.g., 33±4° C. (e.g., 33±3° C. (e.g., 33±2° C. (e.g., 33±1° C. (e.g., 33±0.5° C.))))))) and about 175±20 CC (e.g., 175±10° C. (e.g., 175±5° C. (e.g., 175±4° C. (e.g., 175±3° C. (e.g., 175±2° C. (e.g., 175±1° C. (e.g., 175 t 0.5° C.))))))), as measured by TGA.

In some embodiments, Form J of Compound No. 1 shows a weight loss of approximately 8.2% between about 33° C. and about 175° C., as measured by TGA.

### Form K

### X-Ray Powder Diffraction (XRPD) Characterization

In some embodiments, the morphic form is Form K of Compound No. 1, the solvate thereof, the hydrate thereof, or the pharmaceutically acceptable salt thereof.

In some embodiments, the morphic form is Form K of Compound No. 1.

In some embodiments, the present disclosure provides a form of Compound No. 1 (“Form K”) characterized by an X-ray diffraction (“XRPD”) pattern comprising peaks at 5.2±0.2, 17.0±0.2, and 20.5±0.2°2θ (e.g., 5.2±0.1, 17.0±0.1, and 20.5±0.1°2θ (e.g., 5.2, 17.0, and 20.5°2θ)) using Cu Kα radiation.

In some embodiments, the XRPD pattern of Form K further comprises at least one peak selected from 6.8±0.2, 21.6±0.2, and 22.4±0.2°2θ (e.g., 6.8±0.1, 21.6±0.1, and 22.4±0.1°2θ (e.g., 6.8, 21.6, and 22.4°2θ)) using Cu Kα radiation.

In some embodiments, the XRPD pattern of Form K further comprises at least two peaks selected from 6.8±0.2, 21.6±0.2, and 22.4±0.2°2θ (e.g., 6.8±0.1, 21.6±0.1, and 22.4±0.1°2θ (e.g., 6.8, 21.6, and 22.4°2θ)) using Cu Kα radiation.

In some embodiments, the XRPD pattern of Form K further comprises peaks at 6.8±0.2, 21.6±0.2, and 22.4±0.2°2θ (e.g., 6.8±0.1, 21.6±0.1, and 22.4±0.1°2θ (e.g., 6.8, 21.6, and 22.4°2θ)) using Cu Kα radiation.

In some aspects, the present disclosure provides a form of Compound No. 1 (“Form K”) characterized by an X-ray diffraction (“XRPD”) pattern comprising at least three peaks selected from 5.2±0.2, 6.8±0.2, 17.0±0.2, 20.5±0.2, 21.6±0.2, and 22.4±0.2°2θ (e.g., 5.2±0.1, 6.8±0.1, 17.0±0.1, 20.5±0.1, 21.6±0.1, and 22.4±0.1°2θ (e.g., 5.2, 6.8, 17.0, 20.5, 21.6, and 22.4°2θ)) using Cu Kα radiation.

In some embodiments, the XRPD pattern of Form K comprises at least four peaks selected from 5.2±0.2, 6.8±0.2, 17.0±0.2, 20.5±0.2, 21.6±0.2, and 22.4±0.2°2θ (e.g., 5.2±0.1, 6.8±0.1, 17.0±0.1, 20.5±0.1, 21.6±0.1, and 22.4±0.1°2θ (e.g., 5.2, 6.8, 17.0, 20.5, 21.6, and 22.4°2θ)) using Cu Kα radiation.

In some embodiments, the XRPD pattern of Form K comprises at least five peaks selected from 5.2±0.2, 6.8±0.2, 17.0±0.2, 20.5±0.2, 21.6±0.2, and 22.4±0.2°2θ (e.g., 5.2±0.1, 6.8±0.1, 17.0±0.1, 20.5±0.1, 21.6±0.1, and 22.4±0.1°2θ (e.g., 5.2, 6.8, 17.0, 20.5, 21.6, and 22.4°2θ)) using Cu Ku radiation.

In some embodiments, the XRPD pattern of Form K comprises peaks at 5.2±0.2, 6.8±0.2, 17.0±0.2, 20.5±0.2, 21.6±0.2, and 22.4±0.2°2θ (e.g., 5.2±0.1, 6.8±0.1, 17.0±0.1, 20.5±0.1, 21.6±0.1, and 22.4±0.1°2θ (e.g., 5.2, 6.8, 17.0, 20.5, 21.6, and 22.4°2θ)) using Cu Ku radiation.

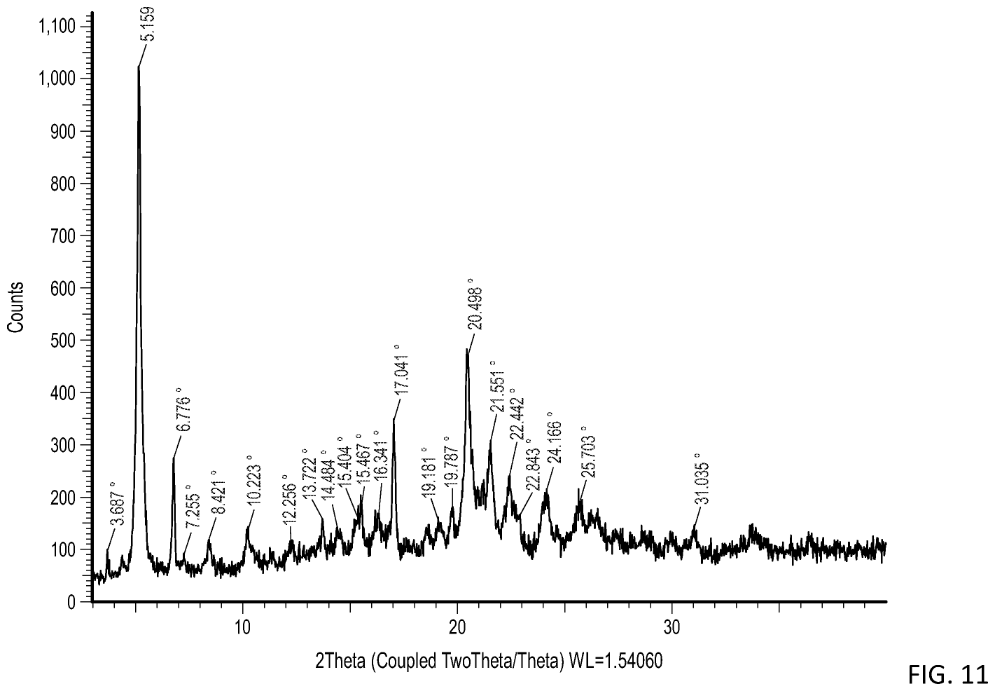

In some embodiments, Form K is characterized by an XRPD pattern substantially similar to that shown in FIG. 11.

In some embodiments, Form K is characterized by an XRPD pattern comprising peaks at approximately the positions shown in the table below. It is understood that the values in the table are approximate and subject to instrumental and experimental variations.

TABLE 11
XRPD peak list for Form K

| | | | Net | Gross | Rel. |
|---|---|---|---|---|---|
| Angle | d Value | | Intensity | Intensity | Intensity |
| 3.687° | 23.94329 | Å | 49.6110 | 96.4141 | 5.2% |
| 5.159° | 17.11537 | Å | 961.265 | 1022.31 | 100.0% |
| 6.776° | 13.03444 | Å | 210.157 | 273.852 | 21.9% |
| 7.255° | 12.17507 | Å | 25.3267 | [symbol] | 2.6% |
| 8.421° | 10.49209 | Å | 57.2274 | 118.000 | 6.0% |
| 10.223° | 8.64597 | Å | 65.4770 | 132.259 | 6.8% |
| 12.256° | 7.21606 | Å | 22.7808 | 98.4288 | 2.4% |
| 13.722° | 6.44794 | Å | 68.8666 | 157.139 | 7.2% |
| 14.484° | 6.11069 | Å | [symbol] | 127.000 | 3.3% |
| 15.404° | 5.74775 | Å | 56.4280 | 157.243 | 5.9% |
| 15.467° | 5.72447 | Å | 64.0079 | 165.020 | 6.7% |
| 16.341° | 5.42006 | Å | 67.4245 | 168.557 | 7.0% |
| 17.041° | 5.19902 | Å | 246.420 | 344.123 | 25.6% |
| 19.181° | 4.62345 | Å | 38.1429 | 147.628 | 4.0% |
| 19.787° | 4.48323 | Å | [symbol] | 175.887 | 6.4% |
| 20.498° | 4.32931 | Å | 351.720 | 469.127 | 36.6% |
| 21.551° | 4.12019 | Å | 190.094 | 305.667 | 19.8% |
| 22.442° | [symbol] | Å | 130.554 | 239.033 | 13.6% |
| 22.843° | [symbol] | Å | [symbol] | 158.701 | 5.7% |
| 24.166° | 3.67979 | Å | 110.466 | 213.476 | 11.5% |
| 25.703° | 3.46322 | Å | 76.8484 | 188.787 | 8.0% |
| 31.035° | [symbol] | Å | 47.7010 | [symbol] | 5.0% |

[symbol]

indicates data missing or illegible when filed

### Form L

### X-Ray Powder Diffraction (XRPD) Characterization

In some embodiments, the morphic form is Form L of Compound No. 1, the solvate thereof, the hydrate thereof, or the pharmaceutically acceptable salt thereof.

In some embodiments, the morphic form is Form L of Compound No. 1.

In some embodiments, the present disclosure provides a form of Compound No. 1 (“Form L”) characterized by an X-ray diffraction (“XRPD”) pattern comprising peaks at 5.4±0.2, 7.8±0.2, and 19.3±0.2°2θ (e.g., 5.4±0.1, 7.8±0.1, and 19.3±0.1°2θ (e.g., 5.4, 7.8, and 19.3°2θ)) using Cu Kα radiation.

In some embodiments, the XRPD pattern of Form L further comprises at least one peak selected from 14.8±0.2, 15.3±0.2, and 16.6±0.2°2θ (e.g., 14.8±0.1, 15.3±0.1, and 16.6±0.1°2θ (e.g., 14.8, 15.3, and 16.6°2θ)) using Cu Kα radiation.

In some embodiments, the XRPD pattern of Form L further comprises at least two peaks selected from 14.8±0.2, 15.3±0.2, and 16.6±0.2°2θ (e.g., 14.8±0.1, 15.3±0.1, and 16.6±0.1°2θ (e.g., 14.8, 15.3, and 16.6°2θ)) using Cu Kα radiation.

In some embodiments, the XRPD pattern of Form L further comprises peaks at 14.8±0.2, 15.3±0.2, and 16.6±0.2°2θ (e.g., 14.8±0.1, 15.3±0.1, and 16.6±0.1°2θ (e.g., 14.8, 15.3, and 16.6°2θ)) using Cu Kα radiation.

In some aspects, the present disclosure provides a form of Compound No. 1 (“Form L”) characterized by an X-ray diffraction (“XRPD”) pattern comprising at least three peaks selected from 5.4±0.2, 7.8±0.2, 14.8±0.2, 15.3±0.2, 16.6±0.2, and 19.3±0.2°2θ (e.g 5.4±0.1, 7.8±0.1, 14.8±0.1, 15.3±0.1, 16.6±0.1, and 19.3±0.1°2θ (e.g., 5.4, 7.8, 14.8, 15.3, 16.6, and 19.3°2θ)) using Cu Kα radiation.

In some embodiments, the XRPD pattern of Form L comprises at least four peaks selected from 5.4±0.2, 7.8±0.2, 14.8±0.2, 15.3±0.2, 16.6±0.2, and 19.3±0.2°2θ (e.g 5.4±0.1, 7.8±0.1, 14.8±0.1, 15.3±0.1, 16.6±0.1, and 19.3±0.1°2θ (e.g., 5.4, 7.8, 14.8, 15.3, 16.6, and 19.3°2θ)) using Cu Kα radiation.

In some embodiments, the XRPD pattern of Form L comprises at least five peaks selected from 5.4±0.2, 7.8±0.2, 14.8±0.2, 15.3±0.2, 16.6±0.2, and 19.3±0.2°2θ (e.g 5.4±0.1, 7.8±0.1, 14.8±0.1, 15.3±0.1, 16.6±0.1, and 19.3±0.1°2θ (e.g., 5.4, 7.8, 14.8, 15.3, 16.6, and 19.3°2θ)) using Cu Kα radiation.

In some embodiments, the XRPD pattern of Form L comprises peaks at 5.4±0.2, 7.8±0.2, 14.8±0.2, 15.3±0.2, 16.6±0.2, and 19.3±0.2°2θ (e.g 5.4±0.1, 7.8±0.1, 14.8±0.1, 15.3±0.1, 16.6±0.1, and 19.3±0.1°2θ (e.g., 5.4, 7.8, 14.8, 15.3, 16.6, and 19.3°2θ)) using Cu Kα radiation.

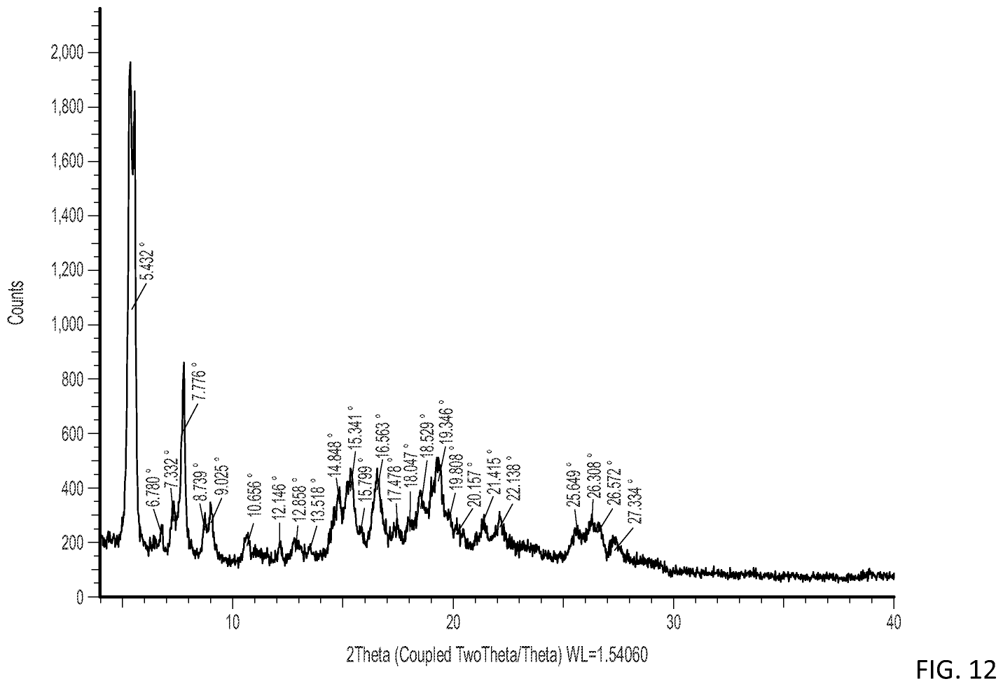

In some embodiments, Form L is characterized by an XRPD pattern substantially similar to that shown in FIG. 12.

In some embodiments, Form L is characterized by an XRPD pattern comprising peaks at approximately the positions shown in the table below. It is understood that the values in the table are approximate and subject to instrumental and experimental variations.

TABLE 12
XRPD peak list for Form L

| | | | Net | Gross | Rel. |
|---|---|---|---|---|---|
| Angle | d Value | | Intensity | Intensity | Intensity |
| 5.432° | 16.25694 | Å | 868.390 | 1055.99 | 100.0% |
| 6.780° | 13.02672 | Å | 58.0653 | 230.273 | 6.7% |
| 7.332° | 12.04687 | Å | 107.793 | 277.411 | 12.4% |
| 7.776° | 11.35962 | Å | 444.419 | [symbol] | 51.2% |
| 8.739° | 10.11051 | Å | 92.8766 | 243.672 | 10.7% |
| 9.025° | 9.79075 | Å | 127.798 | 272.623 | 14.7% |
| 10.656° | 8.29540 | Å | [symbol] | 187.387 | 6.5% |
| 12.146° | 7.28114 | Å | [symbol] | 182.470 | 5.3% |
| [symbol] | [symbol] | Å | [symbol] | 164.903 | 2.8% |
| [symbol] | 6.54519 | Å | [symbol] | 177.579 | 3.7% |
| 14.848° | 5.96162 | Å | 169.578 | 341.525 | 19.5% |
| 15.341° | 5.77090 | Å | 205.275 | 388.787 | 23.6% |
| 15.799° | 5.60455 | Å | [symbol] | [symbol] | 4.3% |
| [symbol] | [symbol] | Å | 189.648 | 392.496 | 21.8% |
| 17.478° | 5.07012 | Å | 49.4221 | 258.090 | 5.7% |
| 18.047° | 4.91141 | Å | 30.9583 | 241.427 | 3.6% |
| 18.529° | 4.78470 | Å | 112.206 | 323.641 | 12.9% |
| 19.346° | 4.58440 | Å | 216.536 | 424.905 | 24.9% |
| 19.808° | 4.47853 | Å | 78.8848 | 282.904 | 9.1% |
| 20.157° | 4.40187 | Å | 30.8917 | 230.375 | 3.6% |
| 21.415° | 4.14592 | Å | 95.0059 | 282.873 | 10.9% |
| 22.133° | 4.01212 | Å | 72.7227 | 255.753 | 8.4% |
| 25.649° | 3.47034 | Å | 90.0076 | 238.539 | 10.4% |
| [symbol] | 3.38494 | Å | 113.293 | 261.937 | 13.0% |
| 26.572° | [symbol] | Å | 87.8553 | 235.467 | 10.1% |
| 27.334° | [symbol] | Å | [symbol] | 169.674 | 3.3% |

[symbol]

indicates data missing or illegible when filed

### Methods of Preparing the Crystalline Forms

In some aspects, the present disclosure features a method of preparing a crystalline form of Compound No. 1 or a pharmaceutically acceptable salt thereof.

In some aspects, the present disclosure provides a method of preparing a crystalline form of Compound No. 1 or a pharmaceutically acceptable salt thereof, comprising one or more steps as described herein.

In some aspects, the present disclosure provides a compound obtainable by, or obtained by, or directly obtained by a method for preparing a crystalline form of Compound No. 1 or a pharmaceutically acceptable salt thereof.

The crystalline form of Compound No. 1 or the pharmaceutically acceptable salt thereof can be prepared by any suitable technique known in the art. Particular processes for the preparation of these compounds are described further in the accompanying examples.

### Pharmaceutical Compositions

In some aspects, the present disclosure features pharmaceutical compositions comprising a morphic form of Compound No. 1, the solvate thereof, the hydrate thereof, or the pharmaceutically acceptable salt thereof described herein, and one or more pharmaceutically acceptable carriers or excipients.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

It may be especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease and also preferably causing complete regression of the disease.

It is understood that the pharmaceutical compositions can be included in a container, pack, or dispenser together with instructions for administration.

### Methods of Use

In some aspects, the present disclosure provides a method of treating or preventing cancer in a subject, comprising administering to the subject a pharmaceutically effective amount of a form of Compound No. 1, the solvate thereof, the hydrate thereof, or the pharmaceutically acceptable salt thereof.

In some aspects, the present disclosure provides a method of treating or preventing cancer in a subject, comprising administering to the subject a pharmaceutically effective amount of a form of Compound No. 1 (e.g., Form A, Form, B, Form C, Form, D, Form E, Form F, Form G, Form H, Form I, Form J, Form K, or Form L), the solvate thereof, the hydrate thereof, or the pharmaceutically acceptable salt thereof.

In some aspects, the present disclosure provides a form of Compound No. 1, the solvate thereof, the hydrate thereof, or the pharmaceutically acceptable salt thereof for use in treating or preventing cancer in a subject.

In some aspects, the present disclosure provides a form of Compound No. 1 (e.g., Form A, Form, B, Form C, Form, D, Form E, Form F, Form G, Form H, Form I, Form J, Form K, or Form L), the solvate thereof, the hydrate thereof, or the pharmaceutically acceptable salt thereof for use in treating or preventing cancer in a subject.

In some aspects, the present disclosure provides use of a form of Compound No. 1, the solvate thereof, the hydrate thereof, or the pharmaceutically acceptable salt thereof, in the manufacture of a medicament for treating or preventing cancer in a subject.

In some aspects, the present disclosure provides use of a form of Compound No. 1 (e.g., Form A, Form, B, Form C, Form, D, Form E, Form F, Form G, Form H, Form I, Form J, Form K, or Form L), the solvate thereof, the hydrate thereof, or the pharmaceutically acceptable salt thereof, in the manufacture of a medicament for treating or preventing cancer in a subject.

In some embodiments, Form A of Compound No. 1, the solvate thereof, the hydrate thereof, or the pharmaceutically acceptable salt thereof is administered.

In some embodiments, Form B of Compound No. 1, the solvate thereof, the hydrate thereof, or the pharmaceutically acceptable salt thereof is administered.

In some embodiments, Form C of Compound No. 1, the solvate thereof, the hydrate thereof, or the pharmaceutically acceptable salt thereof is administered.

In some embodiments, Form D of Compound No. 1, the solvate thereof, the hydrate thereof, or the pharmaceutically acceptable salt thereof is administered.

In some embodiments, Form E of Compound No. 1, the solvate thereof, the hydrate thereof, or the pharmaceutically acceptable salt thereof is administered.

In some embodiments, Form F of Compound No. 1, the solvate thereof, the hydrate thereof, or the pharmaceutically acceptable salt thereof is administered.

In some embodiments, Form G of Compound No. 1, the solvate thereof, the hydrate thereof, or the pharmaceutically acceptable salt thereof is administered.

In some embodiments, Form H of Compound No. 1, the solvate thereof, the hydrate thereof, or the pharmaceutically acceptable salt thereof is administered.

In some embodiments, Form I of Compound No. 1, the solvate thereof, the hydrate thereof, or the pharmaceutically acceptable salt thereof is administered.

In some embodiments, Form J of Compound No. 1, the solvate thereof, the hydrate thereof, or the pharmaceutically acceptable salt thereof is administered.

In some embodiments, Form K of Compound No. 1, the solvate thereof, the hydrate thereof, or the pharmaceutically acceptable salt thereof is administered.

In some embodiments, Form L of Compound No. 1, the solvate thereof, the hydrate thereof, or the pharmaceutically acceptable salt thereof is administered.

### Suitable Subjects and Diseases

In some embodiments, the subject is a mammal.

In some embodiments, the subject is a human.

In some embodiments, the subject is a mouse.

The compounds of the disclosure inhibit or modulate the activity of a receptor tyrosine kinase, in particular extracellular mutants of ErbB-receptors, such as, but not limited to, EGFR-Viii, EGFR-Vii, EGFR-Vvi, EGFR-A289V and EGFR-G598V and HER2-S310F. Thus, the compounds and compositions of the disclosure can be useful as a medicament, i.e. as a medicament in therapy, more specifically for the prevention or treatment of cancer, as detailed below. Therefore, in a further aspect, the present disclosure provides a method of prevention or treatment of a mammal, for example, a human, suffering from cancer, as detailed below.

The term “prevention” or “preventing” refers to reducing or eliminating the onset of the symptoms or complications of a disease (e.g., cancer). Such prevention comprises the step of administering a therapeutically effective amount of a compound of Formula I or salt thereof (or of a pharmaceutical composition containing a compound of Formula 1 or salt thereof) to said mammal, for example, a human.

The term “treatment” or “treating” is intended to encompass therapy and cure. Such treatment comprises the step of administering a therapeutically effective amount of a compound of Formula I or salt thereof (or of a pharmaceutical composition containing a compound of Formula I or salt thereof) to said mammal, for example, a human.

Thus, the disclosure provides the use of the compounds of the disclosure or pharmaceutically acceptable salts or stereoisomers thereof or a pharmaceutical composition thereof for the prevention or treatment of cancer, as detailed below, in a mammal, for example a human.

In some aspects, the present disclosure is directed to a method of inhibiting an oncogenic variant of an ErbB receptor (e.g., an oncogenic variant of an EGFR), comprising administering the subject in need thereof a therapeutically effective amount of a compound described herein.

In some aspects, the present disclosure is directed to a method of inhibiting an oncogenic variant of an ErbB receptor (e.g., an oncogenic variant of an EGFR), comprising administering the subject in need thereof a composition described herein.

In some aspects, the present disclosure is directed to a method of preventing or treating cancer, comprising administering the subject in need thereof a therapeutically effective amount of a compound described herein.

In some aspects, the present disclosure is directed to a method of preventing or treating cancer, comprising administering the subject in need thereof a composition described herein.

In some aspects, the present disclosure is directed to a compound described herein for use in the inhibition of an oncogenic variant of an ErbB receptor (e.g., an oncogenic variant of an EGFR).

In some aspects, the present disclosure is directed to a compound described herein for use in the prevention or treatment of cancer.

In some aspects, the present disclosure is directed to a composition described herein for use in the inhibition of an oncogenic variant of an ErbB receptor (e.g., an oncogenic variant of an EGFR).

In some aspects, the present disclosure is directed to a composition described herein for use in the prevention or treatment of cancer.

In some aspects, the present disclosure is directed to use of a compound described herein in the manufacture of a medicament for inhibiting an oncogenic variant of an ErbB receptor (e.g., an oncogenic variant of an EGFR).

In some aspects, the present disclosure is directed to use of a compound described herein in the manufacture of a medicament for preventing or treating cancer.

In some embodiments, the compound is selected from the compounds described in Tables 1 and 2, pharmaceutically acceptable salts thereof, and stereoisomers thereof.

In some embodiments, the compound is selected from the compounds described in Tables 1 and 2 and pharmaceutically acceptable salts thereof.

In some embodiments, the compound is selected from the compounds described in Tables 1 and 2.

In some embodiments, cancer is a solid tumor.

In some embodiments, the cancer is a bladder cancer, a breast cancer, a cervical cancer, a colorectal cancer, an endometrial cancer, a gastric cancer, a glioblastoma (GBM), a head and neck cancer, a lung cancer, a non-small cell lung cancer (NSCLC), or any subtype thereof.

In some embodiments, the cancer is glioblastoma (GBM) or any subtype thereof.

In some embodiments, the cancer is glioblastoma.

In some embodiments, the cancer, or a tumor or a cell thereof, expresses an oncogenic variant of an ErbB receptor.

In some embodiments, the oncogenic variant of the ErbB receptor comprises an allosteric mutation.

In some embodiments, the oncogenic variant of an ErbB receptor is is an allosteric variant of the ErbB receptor.

In some embodiments, the ErbB receptor is an an epidermal growth factor receptor (EGFR) or a human epidermal growth factor receptor 2 (HER2) receptor.

In some embodiments, the ErbB receptor is an epidermal growth factor receptor (EGFR).

In some embodiments, the ErbB receptor is a HER2 receptor.

In some embodiments, the cancer, or a tumor or a cell thereof, expresses an oncogenic variant of an epidermal growth factor receptor (EGFR).

In some embodiments, the oncogenic variant of EGFR is an allosteric variant of EGFR.

In some embodiments, the oncogenic variant of EGFR comprises an allosteric mutation.

In some embodiments, the cancer, or a tumor or a cell thereof, expresses an oncogenic variant of a HER2 receptor.

In some embodiments, the oncogenic variant of the HER2 receptor is an allosteric variant of the HER2 receptor.

In some embodiments, the oncogenic variant of the HER2 receptor comprises an allosteric mutation.

In some embodiments, the oncogenic variant of an EGFR comprises an EGFR variant III (EGFR-Viii) mutation.

In some embodiments, the oncogenic variant of EGFR comprises an EGFR variant II (EGFR-Vii) mutation.

In some embodiments, the oncogenic variant of EGFR comprises an EGFR variant VI (EGFR-Vvi) mutation.

In some embodiments, the oncogenic variant of an EGFR comprises a substitution of a valine (V) for an alanine (A) at position 289 of SEQ ID NO: 1.

In some embodiments, the oncogenic variant of EGFR comprises a substitution of a valine (V) for a glycine (G) at position 598 of SEQ ID NO: 1.

In some embodiments, the cancer, or a tumor or a cell thereof, expresses an oncogenic variant of an EGFR and wherein the oncogenic variant of EGFR is an allosteric variant of EGFR, the oncogenic variant of an EGFR comprises a modification of a structure of the EGFR, wherein the oncogenic variant of an EGFR is a capable of forming a covalently linked dimer, wherein the covalently linked dimer is constitutively active and wherein the covalently linked dimer enhances an activity of EGFR when contacted to a Type I ErbB inhibitor. In some embodiments, the modification of the structure of the EGFR comprises a modification of one or more of a nucleic acid sequence, an amino acid sequence, a secondary structure, a tertiary structure, and a quaternary structure. In some embodiments, the oncogenic variant comprises a mutation, a splicing event, a post-translational process, a conformational change or any combination thereof. In some embodiments, the modification of the structure of the EGFR occurs within a first cysteine rich (CR1) and/or second cysteine rich (CR2) region of EGFR. In some embodiments, the first cysteine rich (CR1) and/or second cysteine rich (CR2) region of EGFR comprises amino acid residues T211-R334 and/or C526-S645 of SEQ ID NO: 1, respectively. In some embodiments, the oncogenic variant of an EGFR generates a physical barrier to formation of a disulfide bond within the CR1 and/or the CR2 region. In some embodiments, the oncogenic variant of an EGFR removes a physical barrier to formation of a disulfide bond within the CR1 and/or the CR2 region.

In some embodiments, the oncogenic variant of an EGFR comprises one or more free or unpaired Cysteine (C) residues located at a dimer interface of the EGFR. In some embodiments, the oncogenic variant of an EGFR comprises one or more free or unpaired Cysteine (C) residues at a site selected from the group consisting of C190-C199, C194-C207, C215-C223, C219-C231, C232-C240, C236-C248, C251-C260, C264-C291, C295-C307, C311-C326, C329-C333, C506-C515, C510-C523, C526-C535, C539-C555, C558-C571, C562-C579, C582-C591, C595-C617, C620-C628 and C624-C636 according to SEQ ID NO: 1. In some embodiments, the modification occurs within 10 angstroms or less of an intramolecular disulfide bond at a site selected from the group consisting of C190-C199, C194-C207, C215-C223, C219-C231, C232-C240, C236-C248, C251-C260, C264-C291, C295-C307, C311-C326, C329-C333, C506-C515, C510-C523, C526-C535, C539-C555, C558-C571, C562-C579, C582-C591, C595-C617, C620-C628 and C624-C636 according to SEQ ID NO: 1.

In some embodiments, the cancer, or a tumor or a cell thereof, expresses an oncogenic variant of EGFR and the oncogenic variant of EGFR is a mutation of EGFR, a nucleotide sequence encoding the oncogenic variant of an EGFR comprises a deletion or the substitution comprises one or more amino acids that encode an adenosine triphosphate (ATP) binding site. In some embodiments, the ATP binding site comprises amino acids E746 to A750 of SEQ ID NO: 1.

In some embodiments, the ATP binding site or the deletion or substitution thereof comprises K858 of SEQ ID NO: 1. In some embodiments, the deletion comprises K858 of SEQ ID NO: 1. In some embodiments, an arginine (R) is substituted for the lysine (K) at position 858 (K858R) of SEQ ID NO: 1.

In some embodiments, the cancer, or a tumor or a cell thereof, expresses an oncogenic variant of an EGFR and wherein the oncogenic variant of EGFR is an allosteric variant of EGFR, a nucleotide sequence encoding the oncogenic variant of an EGFR comprises an insertion within a sequence encoding exon 20 or a portion thereof. In some embodiments, the sequence encoding exon 20 or a portion thereof comprises a sequence encoding KEILDEAYVMASVDNPHVCAR (SEQ ID NO: 7). In some embodiments, the sequence encoding exon 20 or a portion thereof comprises a sequence encoding a C-helix, a terminal end of the C-helix or a loop following the C-helix. In some embodiments, the insertion comprises the amino acid sequence of ASV, SVD, NPH, or FQEA. In some embodiments, the sequence encoding exon 20 or a portion thereof comprises one or more of: (a) an insertion of the amino acid sequence ASV between positions V769 and D770 of SEQ ID NO: 1; (b) an insertion of the amino acid sequence SVD between positions D770 and N771 of SEQ ID NO: 1; (c) an insertion of the amino acid sequence NPH between positions H773 and V774 of SEQ ID NO: 1; (d) an insertion of the amino acid sequence FQEA between positions A763 and Y764 of SEQ ID NO: 1; (e) an insertion of the amino acid sequence PH between positions H773 and V774 of SEQ ID NO: 1; (f) an insertion of the amino acid G between positions D770 and N771 of SEQ ID NO: 1; (g) an insertion of the amino acid H between positions H773 and V774 of SEQ ID NO: 1; (h) an insertion of the amino acid sequence HV between positions V774 and C775 of SEQ ID NO: 1; (i) an insertion of the amino acid sequence AH between positions H773 and V774 of SEQ ID NO: 1; (j) an insertion of the amino acid sequence SVA between positions A767 and S768 of SEQ ID NO: 1; (k) a substitution of the amino acid sequence GYN for the DN between positions 770 and 771 of SEQ ID NO: 1; (1) an insertion of the amino acid H between positions N771 and P772 of SEQ ID NO: 1; (m) an insertion of the amino acid Y between positions H773 and V774 of SEQ ID NO: 1; (n) an insertion of the amino acid sequence PHVC between positions C775 and R776 of SEQ ID NO: 1; (o) a substitution of the amino acid sequence YNPY for the H at position 773 of SEQ ID NO: 1; (p) an insertion of the amino acid sequence DNP between positions P772 and H773 of SEQ ID NO: 1; (q) an insertion of the amino acid sequence VDS between positions S768 and V769 of SEQ ID NO: 1; (r) an insertion of the amino acid H between positions D770 and N771 of SEQ ID NO: 1; (s) an insertion of the amino acid N between positions N771 and P772 of SEQ ID NO: 1; (t) an insertion of the amino acid sequence PNP between positions P772 and H773 of SEQ ID NO: 1; (u) a substitution of the amino acid sequence GSVDN for the DN between positions 770 and 771 of SEQ ID NO: 1; (v) a substitution of the amino acid sequence GYP for the NP between positions 771 and 772 of SEQ ID NO: 1; (w) an insertion of the amino acid G between positions N771 and P772 of SEQ ID NO: 1; (x) an insertion of the amino acid sequence GNP between positions P772 and H773 of SEQ ID NO: 1; (y) an insertion of the amino acid sequence GSV between positions V769 and D770 of SEQ ID NO: 1; (z) a substitution of the amino acid sequence GNPHVC for the VC between positions 774 and 775 of SEQ ID NO: 1; (aa) an insertion of the amino acid sequence LQEA between positions A763 and Y764 of SEQ ID NO: 1; (bb) an insertion of the amino acid sequence GL between positions D770 and N771 of SEQ ID NO: 1; (cc) an insertion of the amino acid Y between positions D770 and N771 of SEQ ID NO: 1; (dd) an insertion of the amino acid sequence NPY between positions H773 and V774 of SEQ ID NO: 1; (ee) an insertion of the amino acid sequence TH between positions H773 and V774 of SEQ ID NO: 1; (ff) a substitution of the amino acid sequence KGP for the NP between positions 771 and 772 of SEQ ID NO: 1; (gg) a substitution of the amino acid sequence SVDNP for the NP between positions 771 and 772 of SEQ ID NO: 1; (hh) an insertion of the amino acid sequence NN between positions N771 and P772 of SEQ ID NO: 1; (ii) an insertion of the amino acid T between positions N771 and P772 of SEQ ID NO: 1; and (jj) a substitution of the amino acid sequence STLASV for the SV between positions 768 and 769 of SEQ ID NO: 1.

In some embodiments, the cancer, or a tumor or a cell thereof, expresses an oncogenic variant of an EGFR and wherein the oncogenic variant of EGFR is an allosteric variant of EGFR, the oncogenic variant of an EGFR comprises EGFR-Vii, EGFR-Vvi, EGFR-R222C, EGFR-R252C, EGFR-R252P, EGFR-R256Y, EGFR-T263P, EGFR-Y270C, EGFR-A289T, EGFR-A289V, EGFR-A289D, EGFR-H304Y, EGFR-G331R, EGFR-P596S, EGFR-P596L, EGFR-P596R, EGFR-G598V, EGFR-G598A, EGFR-G614D, EGFR-C620Y, EGFR-C614W, EGFR-C628F, EGFR-C628Y, EGFR-C636Y, EGFR-G645C, EGFR-□660, EGFR-□768 or any combination thereof.

In some embodiments, the cancer, or a tumor or a cell thereof, expresses one or more of: (a) a wild type human epidermal growth factor receptor 2 (HER2) receptor or an oncogenic variant of a HER-2 receptor.

In some embodiments, the cancer, or a tumor or a cell thereof, expresses a wild type HER-2 receptor, the wild type HER2 receptor comprises the amino acid sequence of SEQ ID NO: 2, 3, 4, 5, or 6.

In some embodiments, the cancer, or a tumor or a cell thereof, expresses an oncogenic variant of a HER-2 receptor, the oncogenic variant of a HER2 receptor is an allosteric variant of the HER2 receptor.

In some embodiments, the cancer, or a tumor or a cell thereof, expresses an oncogenic variant of a HER-2 receptor and wherein the oncogenic variant of a HER2 receptor is an allosteric variant of the HER2 receptor, the oncogenic variant of a HER2 receptor comprises a substitution of a phenylalanine (F) for a serine (S) at position 310 of SEQ ID NO: 2 or 5.

In some embodiments, the cancer, or a tumor or a cell thereof, expresses an oncogenic variant of a HER-2 receptor and wherein the oncogenic variant of a HER2 receptor is an allosteric variant of the HER2 receptor, the oncogenic variant of a HER2 receptor comprises a substitution of a tyrosine (Y) for a serine (S) at position 310 of SEQ ID NO: 2 or 5.

In some embodiments, the cancer, or a tumor or a cell thereof, expresses an oncogenic variant of a HER-2 receptor and wherein the oncogenic variant of a HER2 receptor is an allosteric variant of the HER2 receptor, the oncogenic variant of a HER2 receptor comprises a substitution of a glutamine (Q) for an arginine (R) at position 678 of SEQ ID NO: 2 or 5.

In some embodiments, the cancer, or a tumor or a cell thereof, expresses an oncogenic variant of a HER-2 receptor and wherein the oncogenic variant of a HER2 receptor is an allosteric variant of the HER2 receptor, the oncogenic variant of a HER2 receptor comprises a substitution of a leucine (L) for a valine (V) at position 777 of SEQ ID NO: 2 or 5.

In some embodiments, the cancer, or a tumor or a cell thereof, expresses an oncogenic variant of a HER-2 receptor and wherein the oncogenic variant of a HER2 receptor is an allosteric variant of the HER2 receptor, the oncogenic variant of a HER2 receptor comprises a substitution of a methionine (M) for a valine (V) at position 777 of SEQ ID NO: 2 or 5.

In some embodiments, the cancer, or a tumor or a cell thereof, expresses an oncogenic variant of a HER-2 receptor and wherein the oncogenic variant of a HER2 receptor is an allosteric variant of the HER2 receptor, the oncogenic variant of a HER2 receptor comprises a substitution of an isoleucine (I) for a valine (V) at position 842 of SEQ ID NO: 2 or 5.

In some embodiments, the cancer, or a tumor or a cell thereof, expresses an oncogenic variant of a HER-2 receptor and wherein the oncogenic variant of a HER2 receptor is an allosteric variant of the HER2 receptor, the oncogenic variant of a HER2 receptor comprises a substitution of an alanine (A) for a leucine (L) at position 755 of SEQ ID NO: 2 or 5.

In some embodiments, the cancer, or a tumor or a cell thereof, expresses an oncogenic variant of a HER-2 receptor and wherein the oncogenic variant of a HER2 receptor is an allosteric variant of the HER2 receptor, the oncogenic variant of a HER2 receptor comprises a substitution of a proline (P) for a leucine (L) at position 755 of SEQ ID NO: 2 or 5.

In some embodiments, the cancer, or a tumor or a cell thereof, expresses an oncogenic variant of a HER-2 receptor and wherein the oncogenic variant of a HER2 receptor is an allosteric variant of the HER2 receptor, the oncogenic variant of a HER2 receptor comprises a substitution of a serine (S) for a leucine (L) at position 755 of SEQ ID NO: 2 or 5.

In some embodiments, the cancer, or a tumor or a cell thereof, expresses an oncogenic variant of a HER-2 receptor and wherein the oncogenic variant of a HER2 receptor is an allosteric variant of the HER2 receptor, a nucleotide sequence encoding the oncogenic variant of a HER2 receptor comprises an insertion within a sequence encoding exon 20 or a portion thereof. In some embodiments, the sequence encoding exon 20 or a portion thereof comprises a sequence encoding KEILDEAYVMAGVGSPYVSR(SEQ ID NO: 8). In some embodiments, the sequence encoding exon 20 or a portion thereof comprises a sequence encoding a C-helix, a terminal end of the C-helix or a loop following the C-helix. In some embodiments, the insertion comprises the amino acid sequence of GSP or YVMA. In some embodiments, the sequence encoding exon 20 or a portion thereof comprises one or more of: (a) an insertion of the amino acid sequence YVMA between positions A775 and G776 of SEQ ID NO: 2; (b) an insertion of the amino acid sequence GSP between positions P780 and Y781 of SEQ ID NO: 2; (c) an insertion of the amino acid sequence YVMA between positions A771 and Y772 of SEQ ID NO: 2; (d) an insertion of the amino acid sequence YVMA between positions A775 and G776 of SEQ ID NO: 2: (e) an insertion of the amino acid V between positions V777 and G778 of SEQ ID NO: 2; (f) an insertion of the amino acid V between positions V777 and G778 of SEQ ID NO: 2; (g) a substitution of the amino acid sequence AVGCV for the GV between positions 776 and 777 of SEQ ID NO: 2; (h) a substitution of the amino acid sequence LC for the G between position 776 of SEQ ID NO: 2; (i) a substitution of the amino acid sequence LCV for the G between position 776 of SEQ ID NO: 2; 0) an insertion of the amino acid sequence GSP between positions V777 and G778 of SEQ ID NO: 2; (k) a substitution of the amino acid sequence PS for the LRE between positions 755 and 757 of SEQ ID NO: 2; (1) a substitution of the amino acid sequence CPGSP for the SP between positions 779 and 780 of SEQ ID NO: 2; (m) an insertion of the amino acid C between positions V777 and G778 of SEQ ID NO: 2; (n) a substitution of the amino acid sequence VVMA for the AG between positions 775 and 776 of SEQ ID NO: 2; (o) a substitution of the amino acid sequence VV for the G at position 776 of SEQ ID NO: 2; (p) a substitution of the amino acid sequence AVCV for the GV between positions 776 and 777 of SEQ ID NO: 2; (q) a substitution of the amino acid sequence VCV for the GV between positions 776 and 777 of SEQ ID NO: 2; (r) an insertion of the amino acid G between positions G778 and S779 of SEQ ID NO: 2; (s) a substitution of the amino acid sequence PK for the LRE between positions 755 and 757 of SEQ ID NO: 2; (t) an insertion of the amino acid V between positions A775 and G776 of SEQ ID NO: 2; (u) an insertion of the amino acid sequence YAMA between positions A775 and G776 of SEQ ID NO: 2; (v) a substitution of the amino acid sequence CV for the G at position 776 of SEQ ID NO: 2; (w) a substitution of the amino acid sequence AVCGG for the GVG between positions 776 and 778 of SEQ ID NO: 2; (x) a substitution of the amino acid sequence CVCG for the GVG between positions 776 and 778 of SEQ ID NO: 2; (y) a substitution of the amino acid sequence VVVG for the GVG between positions 776 and 778 of SEQ ID NO: 2. (z) a substitution of the amino acid sequence SVGG for the GVGS between positions 776 and 779 of SEQ ID NO: 2; (aa) a substitution of the amino acid sequence VVGES for the GVGS between positions 776 and 779 of SEQ ID NO: 2: (bb) a substitution of the amino acid sequence AVGSGV for the GV between positions 776 and 777 of SEQ ID NO: 2; (cc) a substitution of the amino acid sequence CVC for the GV between positions 776 and 777 of SEQ ID NO: 2; (dd) a substitution of the amino acid sequence HVC for the GV between positions 776 and 777 of SEQ ID NO: 2: (ee) a substitution of the amino acid sequence VAAGV for the GV between positions 776 and 777 of SEQ ID NO: 2; (ff) a substitution of the amino acid sequence VAGV for the GV between positions 776 and 777 of SEQ ID NO: 2; (gg) a substitution of the amino acid sequence VVV for the GV between positions 776 and 777 of SEQ ID NO: 2; (hh) an insertion of the amino acid sequence FPG between positions G778 and S779 of SEQ ID NO: 2: (ii) an insertion of the amino acid sequence GS between positions S779 and P780 of SEQ ID NO: 2; (jj) a substitution of the amino acid sequence VPS for the VLRE between positions 754 and 757 of SEQ ID NO: 2; (kk) an insertion of the amino acid E between positions V777 and G778 of SEQ ID NO: 2; (11) an insertion of the amino acid sequence MAGV between positions V777 and G778 of SEQ ID NO: 2; (mm) an insertion of the amino acid S between positions V777 and G778 of SEQ ID NO: 2; (nn) an insertion of the amino acid sequence SCV between positions V777 and G778 of SEQ ID NO: 2; and (oo) an insertion of the amino acid sequence LMAY between positions Y772 and V773 of SEQ ID NO: 2.

In some embodiments, the cancer, or a tumor or a cell thereof, expresses an oncogenic variant of a HER-2 receptor and wherein the oncogenic variant of a HER2 receptor is an allosteric variant of the HER2 receptor, the oncogenic variant of a HER2 receptor comprises HER2-□16, HER2-C311R, HER2-S310F, p95-HER2-M611 or any combination thereof.

In some embodiments, the cancer, or a tumor or a cell thereof, expresses an oncogenic variant of a HER-4 receptor. In some embodiments, the oncogenic variant of the HER-4 receptor is an allosteric variant of the HER4 receptor. In some embodiments, the oncogenic variant of a HER4 receptor comprises deletion of exon 16 (HER4-A16).

In some embodiments, the cancer, or a tumor or a cell thereof, expresses an oncogenic variant of an EGFR, wherein the sequence encoding the oncogenic variant of the EGFR comprises a deletion of exon 20 or a portion thereof and wherein the the cancer, the tumor or the cell thereof does not comprise a second oncogenic variation in a sequence other than exon 20 of EGFR. In some embodiments, the second oncogenic variation comprises a sequence encoding one or more of an EGFR kinase domain (KD), BRAF, NTRK, and KRAS.

In some embodiments, the cancer, or a tumor or a cell thereof, expresses an oncogenic variant of an EGFR, wherein the sequence encoding the oncogenic variant of the EGFR comprises a deletion of exon 20 or a portion thereof and wherein the the cancer, the tumor or the cell thereof does not comprise a marker indicating responsiveness to immunotherapy.

In some embodiments, the oncogenic variant (e.g., allosteric variant) or the oncogenic mutation (e.g., allosteric mutation) is detected by a Food and Drug Administration (FDA)-approved diagnosis.

In some embodiments, prior to the treatment with the compound of the present disclosure, the subject is treated with a therapeutic agent different from the compound of the present disclosure.

In some embodiments, the cancer, or a tumor or a cell thereof, is insensitive or resistant to treatment with a therapeutic agent different from the compound of the present disclosure. In some embodiments, the cancer, or a tumor or a cell thereof, is insensitive or resistant to treatment with a Type I inhibitor. In some embodiments, the cancer, or a tumor or a cell thereof, is insensitive or resistant to treatment with one or more of gefinitinib, erlotinib, afatinib, osimertinib, necitunumab, crizotinib, alectinib, ceritinib, dabrafenib, trametinib, afatinib, sapitinib, dacomitinib, canertinib, pelitinib, WZ4002, WZ8040, WZ3146, CO-1686 and AZD9291.

In some embodiments, the subject has an adverse reaction to treatment with a therapeutic agent different from the compound of the present disclosure. In some embodiments, the subject has an adverse reaction to treatment with a Type I inhibitor. In some embodiments, the subject has an adverse reaction to treatment with one or more of gefinitinib, erlotinib, afatinib, osimertinib, necitunumab, crizotinib, alectinib, ceritinib, dabrafenib, trametinib, afatinib, sapitinib, dacomitinib, canertinib, pelitinib, WZ4002, WZ8040, WZ3146, CO-1686 and AZD9291. In some embodiments, the adverse reaction is an activation of the oncogenic variant of an EGFR and wherein the oncogenic variant comprises a mutation in an extracellular domain of the receptor. In some embodiments, the adverse reaction is an activation of the oncogenic variant of a HER-2 Receptor and wherein the oncogenic variant comprises a mutation in an extracellular domain of the receptor.

In some embodiments, the method further comprises administering to the subject in need thereof a therapeutically effective amount of a non-Type 1 inhibitor. In some embodiments, the non-Type I inhibitor comprises a small molecule Type U inhibitor.

In some embodiments, the method further comprises administering to the subject in need thereof a therapeutically effective amount of a non-Type I inhibitor. In some embodiments, the non-Type I inhibitor comprises a small molecule Type II inhibitor.

In some embodiments, the compound is used in combination with a therapeutically effective amount of a non-Type I inhibitor. In some embodiments, the non-Type I inhibitor comprises a small molecule Type 11 inhibitor.

In some embodiments, the composition further comprises a non-Type I inhibitor. In some embodiments, the non-Type I inhibitor comprises a small molecule Type II inhibitor.

In some embodiments, the therapeutically effective amount reduces a severity of a sign or symptom of the cancer.

In some embodiments, the sign of the cancer comprises a tumor grade and wherein a reduction of the severity of the sign comprises a decrease of the tumor grade.

In some embodiments, the sign of the cancer comprises a tumor metastasis and wherein a reduction of the severity of the sign comprises an elimination of the metastasis or a reduction in the rate or extent the metastasis.

In some embodiments, the sign of the cancer comprises a tumor volume and wherein a reduction of the severity of the sign comprises an elimination of the tumor or a reduction in the volume.

In some embodiments, the symptom of the cancer comprises pain and wherein a reduction of the severity of the sign comprises an elimination or a reduction in the pain.

In some embodiments, the therapeutically effective amount induces a period of remission.

In some embodiments, the therapeutically effective amount improves a prognosis of the subject.

Such a use (or method of prevention or treatment) of a subject comprises administering to a subject in need of such prevention or treatment a therapeutically effective amount of a compound of the disclosure or pharmaceutically acceptable salts thereof or a pharmaceutical composition thereof by targeting allosteric and/or oncogenic variants of EGFR and HER-2 receptor.

### Definitions

It is understood that the compounds described herein include the compounds themselves, as well as their salts, and their solvates, if applicable. A salt, for example, can be formed between an anion and a positively charged group (e.g., amino) on a substituted benzene compound.

Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

Unless explicitly indicated otherwise, the terms “approximately” and “about” are synonymous. In some embodiments, “approximately” and “about” refer to the recited amount, value, dose, or duration±20%, ±15%, ±10%, +8%, ±6%, ±5%, ±4%, ±2%, ±1%, or ±0.5%.

In some embodiments, “approximately” and “about” refer to the listed amount or duration±10%, ±8%, ±6%, ±5%, ±4%, or ±2%. In some embodiments, “approximately” and “about” refer to the listed amount, value, dose, or duration±5%. In some embodiments, “approximately” and “about” refer to the listed amount, value, dose, or duration±2%. In some embodiments, “approximately” and “about” refer to the listed amount, value, dose, or duration±1%.

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted benzene compound.

Suitable cations include sodium ion, potassium ion, magnesium ion, calcium ion, and an ammonium cation such as tetramethylammonium ion. The substituted benzene compounds also include those salts containing quaternary nitrogen atoms.

It is understood that the compounds of the present disclosure, for example, the salts of the compounds, can exist in either hydrated or unhydrated (the anhydrous) form or as solvates with other solvent molecules. Nonlimiting examples of hydrates include monohydrates and dihydrates. Nonlimiting examples of solvates include ethanol solvates and acetone solvates.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March 's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art.

It is to be understood that, unless otherwise stated, any description of a method of treatment or prevention includes use of a crystalline form of Compound No. 1 or a pharmaceutically acceptable salt thereof to provide such treatment or prevention as is described herein. It is to be further understood, unless otherwise stated, any description of a method of treatment or prevention includes use of a crystalline form of Compound No. 1 or a pharmaceutically acceptable salt thereof to prepare a medicament to treat or prevent such condition. The treatment or prevention includes treatment or prevention of human or non-human animals including rodents and other disease models.

It is to be understood that, unless otherwise stated, any description of a method of treatment includes use of a crystalline form of Compound No. 1 or a pharmaceutically acceptable salt thereof to provide such treatment as is described herein. It is to be further understood, unless otherwise stated, any description of a method of treatment includes use of a crystalline form of Compound No. 1 or a pharmaceutically acceptable salt thereof to prepare a medicament to treat such condition. The treatment includes treatment of human or non-human animals including rodents and other disease models.

As used herein, the term “subject” refers to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human.

In some embodiments, the term “subject in need thereof” can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject in need thereof may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model.

It is to be understood that a crystalline form of Compound No. 1 or a pharmaceutically acceptable salt thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that “solubility” or “solubility rating” refers to the property of a polymorph (e.g., Form A, B, C, D, E, F, G, H, I, J, K, or L) disclosed herein to dissolve in a liquid solvent and form a homogeneous solution. In some embodiments, solubility is expressed as a concentration, either by mass of solute per unit volume of solvent (e.g., g of solute per kg of solvent, g per dL (100 mL), mg/ml, etc.), molarity, molality, mole fraction, or other similar descriptions of concentration. A person of skill in the art may understand that the maximum equilibrium amount of solute that can dissolve per amount of solvent is the solubility of that solute in that solvent under the specified conditions, including temperature, pressure, pH, and the nature of the solvent.

As used herein, “stable” refers to a polymorph that maintains purity, appearance, and/or analytical parameters over a defined time and temperature as compared to the polymorph as isolated. In some embodiments, the “stable” polymorph exhibits less than about 100/, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% impurity over a set period of time (e.g., 1 day, 2 days, 3 days, 4 days, 5 days, 6 days, one week, two weeks, three weeks, one month, two months, three months, or four months).

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. In some embodiments, the pharmaceutically acceptable salt of a compound is also a prodrug of the compound. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ration other than 1:1, e.g., 3:1, 2:1, 1:2, or 1:3.

It is understood that all references to pharmaceutically acceptable salts include solvent addition forms (solvates) or crystal forms (polymorphs) as defined herein, of the same salt.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXEMPLARY EMBODIMENTS

Embodiment 1. A morphic form of Compound No. 1, a solvate thereof, a hydrate thereof, or a pharmaceutically acceptable salt thereof.

Embodiment 2. The morphic form of embodiment 1, wherein the morphic form is a crystalline form.

Embodiment 3. The morphic form of embodiment 1 or 2, wherein the morphic form is Form A, the solvate thereof, the hydrate thereof, or the pharmaceutically acceptable salt thereof.

Embodiment 4. The morphic form of embodiment 3, wherein Form A is characterized by an X-ray diffraction (“XRPD”) pattern comprising peaks at 4.8±0.2, 5.6±0.2, and 17.6±0.2°2θ (e.g., 4.8±0.1, 5.6±0.1, and 17.6±0.1°2θ (e.g., 4.8, 5.6, and 17.6°2θ)) using Cu Kα radiation.

Embodiment 5. The morphic form of embodiment 4, wherein Form A further comprises at least one peak selected from 9.6±0.2, 20.0±0.2, and 24.6±0.2°2θ (e.g., 9.6±0.1, 20.0±0.1, and 24.6±0.1°2θ (e.g., 9.6, 20.0, and 24.6°2θ)) using Cu Kα radiation.

Embodiment 6. The morphic form of embodiment 3, wherein Form A is characterized by an X-ray diffraction (“XRPD”) pattern comprising at least three peaks selected from 4.8±0.2, 5.6±0.2, 9.6±0.2, 17.6±0.2, 20.0±0.2, and 24.6±0.2°2θ (e.g., 4.8±0.1, 5.6±0.1, 9.6±0.1, 17.6±0.1, 20.0±0.1, and 24.6±0.1°2θ (e.g., 4.8, 5.6, 9.6, 17.6, 20.0, and 24.6°2θ)) using Cu Kα radiation.

Embodiment 7. The morphic form of embodiment 3, wherein Form A is characterized by an XRPD pattern substantially similar to that shown in FIG. 1.

Embodiment 8. The morphic form of embodiment 3, wherein Form A is characterized by a DSC curve having at least one endothermic peak selected from 76±20, 141±20, 156±20, and 170±20° C. (e.g., 76±10, 141±10, 156±10, and 170±10° C. (e.g., 76±5, 141±5, 156±5, and 170±5° C. (e.g., 76±4, 141±4, 156±4, and 170±4° C. (e.g., 76±3, 141±3, 156±3, and 170±3° C. (e.g., 76±2, 141±2, 156±2, and 170±2° C. (e.g., 76±1, 141±1, 156±1, and 170±1° C. (e.g., 76±0.5, 141±0.5, 156±0.5, and 170±0.5° C.))))))).

Embodiment 9. The morphic form of embodiment 1 or 2, wherein the morphic form is Form B, the solvate thereof, the hydrate thereof, or the pharmaceutically acceptable salt thereof.

Embodiment 10. The morphic form of embodiment 9, wherein Form B is characterized by an X-ray diffraction (“XRPD”) pattern comprising peaks at 6.5±0.2, 16.7±0.2, and 18.6±0.2°2θ (e.g., 6.5±0.1, 16.7±0.1, and 18.6±0.1°2θ (e.g., 6.5, 16.7, and 18.6°2θ)) using Cu Kα radiation.

Embodiment 11. The morphic form of embodiment 10, wherein Form B further comprises at least one peak selected from 3.2±0.2, 13.4±0.2, and 17.7±0.2°2θ (e.g., 3.2±0.1, 13.4±0.1, and 17.7±0.1°2θ (e.g., 3.2, 13.4, and 17.7°2θ)) using Cu Kα radiation.

Embodiment 12. The morphic form of embodiment 9, wherein Form B is characterized by an X-ray diffraction (“XRPD”) pattern comprising at least three peaks selected from 3.2±0.2, 6.5±0.2, 13.4±0.2, 16.7±0.2, 17.7±0.2, and 18.6±0.2°2θ (e.g., 3.2±0.1, 6.5±0.1, 13.4±0.1, 16.7±0.1, 17.7±0.1, and 18.6±0.1°2θ (e.g., 3.2, 6.5, 13.4, 16.7, 17.7, and 18.6°2θ)) using Cu Kα radiation.

Embodiment 13. The morphic form of embodiment 9, wherein Form B is characterized by an XRPD pattern substantially similar to that shown in FIG. 2.

Embodiment 14. The morphic form of embodiment 9, wherein Form B is characterized by a DSC curve having at least one endothermic peak selected from 155±20 and 176±20° C. (e.g., 155±10 and 176±10° C. (e.g., 155±5 and 176±5° C. (e.g., 155±4 and 176±4° C. (e.g., 155±3 and 176±3° C. (e.g., 155±2 and 176±2° C. (e.g., 155±1 and 176±1° C. (e.g., 155±0.5 and 176±0.5° C.))))))).

Embodiment 15. The morphic form of embodiment 1 or 2, wherein the morphic form is Form C, the solvate thereof, the hydrate thereof, or the pharmaceutically acceptable salt thereof.

Embodiment 16. The morphic form of embodiment 15, wherein Form C is characterized by an X-ray diffraction (“XRPD”) pattern comprising peaks at 16.1±0.2, 16.7±0.2, and 19.1±0.2°2θ (e.g., 16.1±0.1, 16.7±0.1, and 19.1±0.1°2θ (e.g., 16.1, 16.7, and 19.1°2θ)) using Cu Kα radiation.

Embodiment 17. The morphic form of embodiment 16, wherein Form C further comprises at least one peak selected from 4.0±0.2, 12.6±0.2, and 14.1±0.2°2θ (e.g., 4.0±0.1, 12.6±0.1, and 14.1±0.1°2θ (e.g., 4.0, 12.6, and 14.1°2θ)) using Cu Kα radiation.

Embodiment 18. The morphic form of embodiment 15, wherein Form C is characterized by an X-ray diffraction (“XRPD”) pattern comprising at least three peaks selected from 4.0±0.2, 12.6±0.2, 14.1±0.2, 16.1±0.2, 16.7±0.2, and 19.1±0.2°2θ (e.g., 4.0±0.1, 12.6±0.1, 14.1±0.1, 16.1±0.1, 16.7±0.1, and 19.1±0.1°2θ (e.g., 4.0, 12.6, 14.1, 16.1, 16.7, and 19.1°2θ)) using Cu Kα radiation.

Embodiment 19. The morphic form of embodiment 15, wherein Form C is characterized by an XRPD pattern substantially similar to that shown in FIG. 3.

Embodiment 20. The morphic form of embodiment 15, wherein Form C is characterized by a DSC curve having an endothermic peak at 181±20° C. (e.g., 181±10° C. (e.g., 181±5° C. (e.g., 181±4° C. (e.g., 181±3° C. (e.g., 181±2° C. (e.g., 181±1° C. (e.g., 181±0.5° C.))))))).

Embodiment 21. The morphic form of embodiment 1 or 2, wherein the morphic form is Form D, the solvate thereof, the hydrate thereof, or the pharmaceutically acceptable salt thereof.

Embodiment 22. The morphic form of embodiment 21, wherein Form D is characterized by an X-ray diffraction (“XRPD”) pattern comprising peaks at 15.3±0.2, 19.3±0.2, and 23.5±0.2°2θ (e.g., 5.3±0.1, 19.3±0.1, and 23.5±0.1°2θ (e.g., 5.3, 19.3, and 23.5°2θ)) using Cu Kα radiation.

Embodiment 23. The morphic form of embodiment 22, wherein Form D further comprises at least one peak selected from 5.4±0.2, 9.7±0.2, and 18.8±0.2°2θ (e.g., 5.4±0.1, 9.7±0.1, and 18.8±0.1°2θ (e.g., 5.4, 9.7, and 18.8°2θ)) using Cu Kα radiation.

Embodiment 24. The morphic form of embodiment 21, wherein Form D is characterized by an X-ray diffraction (“XRPD”) pattern comprising at least three peaks selected from 5.4±0.2, 9.7±0.2, 15.3±0.2, 18.8±0.2, 19.3±0.2, and 23.5±0.2°2θ (e.g., 5.4±0.1, 9.7±0.1, 15.3±0.1, 18.8±0.1, 19.3±0.1, and 23.5±0.1°2θ (e.g., 5.4, 9.7, 15.3, 18.8, 19.3, and 23.5°2θ)) using Cu Kα radiation.

Embodiment 25. The morphic form of embodiment 21, wherein Form D is characterized by an XRPD pattern substantially similar to that shown in FIG. 4.

Embodiment 26. The morphic form of embodiment 21, wherein Form D is characterized by a DSC curve having at least one endothermic peak selected from 128±20, 149±20, and 175±20° C. (e.g., 128±10, 149±10, and 175±10° C. (e.g., 128±5, 149±5, and 175±5° C. (e.g., 128±4, 149±4, and 175±4° C. (e.g., 128±3, 149±3, and 175±3° C. (e.g., 128±2, 149±2, and 175±2° C. (e.g., 128±1, 149±1, and 175±1° C. (e.g., 128±0.5, 149±0.5, and 175±0.5° C.))))))).

Embodiment 27. The morphic form of embodiment 1 or 2, wherein the morphic form is Form E, the solvate thereof, the hydrate thereof, or the pharmaceutically acceptable salt thereof.

Embodiment 28. The morphic form of embodiment 27, wherein Form E is characterized by an X-ray diffraction (“XRPD”) pattern comprising peaks at 7.4±0.2, 15.8±0.2, and 16.3±0.2°2θ (e.g., 7.4±0.1, 15.8±0.1, and 16.3±0.1°2θ (e.g., 7.4, 15.8, and 16.3°2θ)) using Cu Kα radiation.

Embodiment 29. The morphic form of embodiment 28, wherein Form E further comprises at least one peak selected from 13.3±0.2, 19.2±0.2, and 22.4±0.2°2θ (e.g., 13.3±0.1, 19.2±0.1, and 22.4±0.1°2θ (e.g., 13.3, 19.2, and 22.4°2θ)) using Cu Kα radiation.

Embodiment 30. The morphic form of embodiment 27, wherein Form E is characterized by an X-ray diffraction (“XRPD”) pattern comprising at least three peaks selected from 7.4±0.2, 13.3±0.2, 15.8±0.2, 16.3±0.2, 19.2±0.2, and 22.4±0.2°2θ (e.g., 7.4±0.1, 13.3±0.1, 15.8±0.1, 16.3±0.1, 19.2±0.1, and 22.4±0.1°2θ (e.g., 7.4, 13.3, 15.8, 16.3, 19.2, and 22.4°2θ)) using Cu Kα radiation.

Embodiment 31. The morphic form of embodiment 27, wherein Form E is characterized by an XRPD pattern substantially similar to that shown in FIG. 5.

Embodiment 32. The morphic form of embodiment 27, wherein Form E is characterized by a DSC curve having an endothermic peak at 173±20° C. (e.g., 173±10° C. (e.g., 173±5° C. (e.g., 173±4° C. (e.g., 173±3° C. (e.g., 173±2° C. (e.g., 173±1° C. (e.g., 173±0.5° C.))))))).

Embodiment 33. The morphic form of embodiment 1 or 2, wherein the morphic form is Form F, the solvate thereof, the hydrate thereof, or the pharmaceutically acceptable salt thereof.

Embodiment 34. The morphic form of embodiment 33, wherein Form F is characterized by an X-ray diffraction (“XRPD”) pattern comprising peaks at 5.5±0.2, 16.4±0.2, and 21.9±0.2°2θ (e.g., 5.5±0.1, 16.4±0.1, and 21.9±0.1°2θ (e.g., 5.5, 16.4, and 21.9°2θ)) using Cu Kα radiation.

Embodiment 35. The morphic form of embodiment 34, wherein Form F further comprises at least one peak selected from 7.2±0.2, 14.5±0.2, and 15.2±0.2°2θ (e.g., 7.2±0.1, 14.5±0.1, and 15.2±0.1°2θ (e.g., 7.2, 14.5, and 15.2°2θ)) using Cu Kα radiation.

Embodiment 36. The morphic form of embodiment 33, wherein Form F is characterized by an X-ray diffraction (“XRPD”) pattern comprising at least three peaks selected from 5.5±0.2, 7.2±0.2, 14.5±0.2, 15.2±0.2, 16.4±0.2, and 21.9±0.2°2θ (e.g., 5.5±0.1, 7.2±0.1, 14.5±0.1, 15.2±0.1, 16.4±0.1, and 21.9±0.1°2θ (e.g., 5.5, 7.2, 14.5, 15.2, 16.4, and 21.9°2θ)) using Cu Kα radiation.

Embodiment 37. The morphic form of embodiment 33, wherein Form F is characterized by an XRPD pattern substantially similar to that shown in FIG. 6.

Embodiment 38. The morphic form of embodiment 33, wherein Form F is characterized by a DSC curve having at least one endothermic peak selected from 48±20, 80±20, 146±20, and 174±20° C. (e.g., 48±10, 80±10, 146±10, and 174±10° C. (e.g., 48±5, 80±5, 146±5, and 174±5° C. (e.g., 48±4, 80±4, 146±4, and 174±4° C. (e.g., 48±3, 80±3, 146±3, and 174±3° C. (e.g., 48±2, 80±2, 146±2, and 174±2° C. (e.g., 48±1, 80±1, 146±1, and 174±1° C. (e.g., 48±0.5, 80±0.5, 146±0.5, and 174±0.5° C.))))))).

Embodiment 39. The morphic form of embodiment 1 or 2, wherein the morphic form is Form G, the solvate thereof, the hydrate thereof, or the pharmaceutically acceptable salt thereof.

Embodiment 40. The morphic form of embodiment 39, wherein Form G is characterized by an X-ray diffraction (“XRPD”) pattern comprising peaks at 4.0±0.2, 5.3±0.2, and 16.0±0.2°2θ (e.g., 4.0±0.1, 5.3±0.1, and 16.0±0.1°2θ (e.g., 4.0, 5.3, and 16.0°2θ)) using Cu Kα radiation.

Embodiment 41. The morphic form of embodiment 40, wherein Form G further comprises at least one peak selected from 7.1±0.2, 16.7±0.2, and 19.2±0.2°2θ (e.g., 7.1±0.1, 16.7±0.1, and 19.2±0.1°2θ (e.g., 7.1, 16.7, and 19.2°2θ)) using Cu Kα radiation.

Embodiment 42. The morphic form of embodiment 39, wherein Form G is characterized by an X-ray diffraction (“XRPD”) pattern comprising at least three peaks selected from 4.0±0.2, 5.3±0.2, 7.1±0.2, 16.0±0.2, 16.7±0.2, and 19.2±0.2°2θ (e.g., 4.0±0.1, 5.3±0.1, 7.1±0.1, 16.0±0.1, 16.7±0.1, and 19.2±0.1°2θ (e.g., 4.0, 5.3, 7.1, 16.0, 16.7, and 19.2°2θ)) using Cu Kα radiation.

Embodiment 43. The morphic form of embodiment 39, wherein Form G is characterized by an XRPD pattern substantially similar to that shown in FIG. 7.

Embodiment 44. The morphic form of embodiment 39, wherein Form G is characterized by a DSC curve having at least one endothermic peak selected from 34±20, 175±20, and 182±20° C. (e.g., 34±10, 175±10, and 182±10° C. (e.g., 34±5, 175±5, and 182±5° C. (e.g., 34±4, 175±4, and 182±4° C. (e.g., 34±3, 175±3, and 182±3° C. (e.g., 34±2, 175±2, and 182±2° C. (e.g., 34±1, 175±1, and 182±1° C. (e.g., 34±0.5, 175±0.5, and 182±0.5° C.))))))).

Embodiment 45. The morphic form of embodiment 1 or 2, wherein the morphic form is Form H, the solvate thereof, the hydrate thereof, or the pharmaceutically acceptable salt thereof.

Embodiment 46. The morphic form of embodiment 45, wherein Form H is characterized by an X-ray diffraction (“XRPD”) pattern comprising peaks at 4.6±0.2, 13.8±0.2, and 17.0±0.2°2θ (e.g., 4.6±0.1, 13.8±0.1, and 17.0±0.1°2θ (e.g., 4.6, 13.8, and 17.0°2θ)) using Cu Kα radiation.

Embodiment 47. The morphic form of embodiment 46, wherein Form H further comprises at least one peak selected from 5.6±0.2, 8.6±0.2, and 17.8±0.2°2θ (e.g., 5.6±0.1, 8.6±0.1, and 17.8±0.1°2θ (e.g., 5.6, 8.6, and 17.8°2θ)) using Cu Kα radiation.

Embodiment 48. The morphic form of embodiment 45, wherein Form H is characterized by an X-ray diffraction (“XRPD”) pattern comprising at least three peaks selected from 4.6±0.2, 5.6±0.2, 8.6±0.2, 13.8±0.2, 17.0±0.2, and 17.8±0.2°2θ (e.g., 4.6±0.1, 5.6±0.1, 8.6±0.1, 13.8±0.1, 17.0±0.1, and 17.8±0.1°2θ (e.g., 4.6, 5.6, 8.6, 13.8, 17.0, and 17.8°2θ)) using Cu Kα radiation.

Embodiment 49. The morphic form of embodiment 45, wherein Form H is characterized by an XRPD pattern substantially similar to that shown in FIG. 8.

Embodiment 50. The morphic form of embodiment 45, wherein Form H is characterized by a DSC curve having at least one endothermic peak selected from 62±20 and 153±20° C. (e.g., 62±10 and 153±10° C. (e.g., 62±5 and 153±5° C. (e.g., 62±4 and 153±4° C. (e.g., 62±3 and 153±3° C. (e.g., 62±2 and 153±2° C. (e.g., 62±1 and 153±1° C. (e.g., 62±0.5 and 153±0.5° C.))))))).

Embodiment 51. The morphic form of embodiment 1 or 2, wherein the morphic form is Form I, the solvate thereof, the hydrate thereof, or the pharmaceutically acceptable salt thereof.

Embodiment 52. The morphic form of embodiment 51, wherein Form I is characterized by an X-ray diffraction (“XRPD”) pattern comprising peaks at 5.1±0.2, 20.4±0.2, and 21.5±0.2°2θ (e.g., 5.1±0.1, 20.4±0.1, and 21.5±0.1°2θ (e.g., 5.1, 20.4, and 21.5°2θ)) using Cu Kα radiation.

Embodiment 53. The morphic form of embodiment 52, wherein Form I further comprises at least one peak selected from 17.0±0.2, 22.3±0.2, and 25.5±0.2°2θ (e.g., 17.0±0.1, 22.3±0.1, and 25.5±0.1°2θ (e.g., 17.0, 22.3, and 25.5°2θ)) using Cu Kα radiation.

Embodiment 54. The morphic form of embodiment 51, wherein Form I is characterized by an X-ray diffraction (“XRPD”) pattern comprising at least three peaks selected from 5.1±0.2, 17.0±0.2, 20.4±0.2, 21.5±0.2, 22.3±0.2, and 25.5±0.2°2θ (e.g., 5.1±0.1, 17.0±0.1, 20.4±0.1, 21.5±0.1, 22.3±0.1, and 25.5±0.1°2θ (e.g., 5.1, 17.0, 20.4, 21.5, 22.3, and 25.5°2θ)) using Cu Kα radiation.

Embodiment 55. The morphic form of embodiment 51, wherein Form I is characterized by an XRPD pattern substantially similar to that shown in FIG. 9.

Embodiment 56. The morphic form of embodiment 1 or 2, wherein the morphic form is Form J, the solvate thereof, the hydrate thereof, or the pharmaceutically acceptable salt thereof.

Embodiment 57. The morphic form of embodiment 56, wherein Form J is characterized by an X-ray diffraction (“XRPD”) pattern comprising peaks at 4.5±0.2, 17.9±0.2, and 22.5±0.2°2θ (e.g., 4.5±0.1, 17.9±0.1, and 22.5±0.1°2θ (e.g., 4.5, 17.9, and 22.5°2θ)) using Cu Kα radiation.

Embodiment 58. The morphic form of embodiment 57, wherein Form J further comprises at least one peak selected from 5.1±0.2, 10.4±0.2, and 16.0±0.2°2θ (e.g., 5.1±0.1, 10.4±0.1, and 16.0±0.1°2θ (e.g., 5.1, 10.4, and 16.0°2θ)) using Cu Kα radiation.

Embodiment 59. The morphic form of embodiment 56, wherein Form J is characterized by an X-ray diffraction (“XRPD”) pattern comprising at least three peaks selected from 4.5±0.2, 5.1±0.2, 10.4±0.2, 16.0±0.2, 17.9±0.2, and 22.5±0.2°2θ (e.g., 4.5±0.1, 5.1±0.1, 10.4±0.1, 16.0±0.1, 17.9±0.1, and 22.5±0.1°2θ (e.g., 4.5, 5.1, 10.4, 16.0, 17.9, and 22.5°2θ)) using Cu Kα radiation.

Embodiment 60. The morphic form of embodiment 59, wherein Form J is characterized by an XRPD pattern substantially similar to that shown in FIG. 10.

Embodiment 61. The morphic form of embodiment 56, wherein Form J is characterized by a DSC curve having at least one endothermic peak selected from 137±20 and 166±20° C. (e.g., 137±10 and 166±10° C. (e.g., 137±5 and 166±5° C. (e.g., 137±4 and 166±4° C. (e.g., 137±3 and 166±3° C. (e.g., 137±2 and 166±2° C. (e.g., 137±1 and 166±1° C. (e.g., 137±0.5 and 166±0.5° C.))))))).

Embodiment 62. The morphic form of embodiment 1 or 2, wherein the morphic form is Form K, the solvate thereof, the hydrate thereof, or the pharmaceutically acceptable salt thereof.

Embodiment 63. The morphic form of embodiment 62, wherein Form K is characterized by an X-ray diffraction (“XRPD”) pattern comprising peaks at 5.2±0.2, 17.0±0.2, and 20.5±0.2°2θ (e.g., 5.2±0.1, 17.0±0.1, and 20.5±0.1°2θ (e.g., 5.2, 17.0, and 20.5°2θ)) using Cu Kα radiation.

Embodiment 64. The morphic form of embodiment 63, wherein Form K further comprises at least one peak selected from 6.8±0.2, 21.6±0.2, and 22.4±0.2°2θ (e.g., 6.8±0.1, 21.6±0.1, and 22.4±0.1°2θ (e.g., 6.8, 21.6, and 22.4°2θ)) using Cu Kα radiation.

Embodiment 65. The morphic form of embodiment 62, wherein Form K is characterized by an X-ray diffraction (“XRPD”) pattern comprising at least three peaks selected from 5.2±0.2, 6.8±0.2, 17.0±0.2, 20.5±0.2, 21.6±0.2, and 22.4±0.2°2θ (e.g., 5.2±0.1, 6.8±0.1, 17.0±0.1, 20.5±0.1, 21.6±0.1, and 22.4±0.1°2θ (e.g., 5.2, 6.8, 17.0, 20.5, 21.6, and 22.4°2θ)) using Cu Kα radiation.

Embodiment 66. The morphic form of embodiment 62, wherein Form K is characterized by an XRPD pattern substantially similar to that shown in FIG. 11.

Embodiment 67. The morphic form of embodiment 1 or 2, wherein the morphic form is Form L, the solvate thereof, the hydrate thereof, or the pharmaceutically acceptable salt thereof.

Embodiment 68. The morphic form of embodiment 67, wherein Form L is characterized by an X-ray diffraction (“XRPD”) pattern comprising peaks at 5.4±0.2, 7.8±0.2, and 19.3±0.2°2θ (e.g., 5.4±0.1, 7.8±0.1, and 19.3±0.1°2θ (e.g., 5.4, 7.8, and 19.3°2θ)) using Cu Kα radiation.

Embodiment 69. The morphic form of embodiment 68, wherein Form L further comprises at least one peak selected from 14.8±0.2, 15.3±0.2, and 16.6±0.2°2θ (e.g., 14.8±0.1, 15.3±0.1, and 16.6±0.1°2θ (e.g., 14.8, 15.3, and 16.6°2θ)) using Cu Kα radiation.

Embodiment 70. The morphic form of embodiment 67, wherein Form L is characterized by an X-ray diffraction (“XRPD”) pattern comprising at least three peaks selected from 5.4±0.2, 7.8±0.2, 14.8±0.2, 15.3±0.2, 16.6±0.2, and 19.3±0.2°2θ (e.g 5.4±0.1, 7.8±0.1, 14.8±0.1, 15.3±0.1, 16.6±0.1, and 19.3±0.1°2θ (e.g., 5.4, 7.8, 14.8, 15.3, 16.6, and 19.3°2θ)) using Cu Kα radiation.

Embodiment 71. The morphic form of embodiment 67, wherein Form L is characterized by an XRPD pattern substantially similar to that shown in FIG. 12.

Embodiment 72. The morphic form of any preceding embodiments, which is at least 90, 95, 96, 97, 98, or 99% pure.

Embodiment 73. A pharmaceutical composition comprising a therapeutically effective amount of any one, or combination, of the morphic forms of any preceding embodiments and a pharmaceutically acceptable excipient.

Embodiment 74. A method of inhibiting an oncogenic variant of an ErbB receptor, comprising administering to the subject in need thereof a therapeutically effective amount of the morphic form of any one of the preceding embodiments.

Embodiment 75. A method of inhibiting an oncogenic variant of an ErbB receptor, comprising administering to the subject in need thereof the pharmaceutical composition of any one of the preceding embodiments.

Embodiment 76. A method of preventing or treating cancer, comprising administering to the subject in need thereof a therapeutically effective amount of the morphic form of any one of the preceding embodiments.

Embodiment 77. A method of preventing or treating cancer, comprising administering to the subject in need thereof the pharmaceutical composition of any one of the preceding embodiments.

Embodiment 78. A method of preventing or treating cancer, comprising: i) identifying a subject candidate as the subject in need of the treatment when that at least one oncogenic variant of an ErbB receptor is present in the subject; and ii) administering to the subject in need of the treatment a therapeutically effective amount of the morphic form of any one of the preceding embodiments.

Embodiment 79. A method of preventing or treating cancer, comprising: i) identifying a subject candidate as the subject in need of the treatment when that at least one oncogenic variant of an ErbB receptor is present in the subject; and ii) administering to the subject in need of the treatment the pharmaceutical composition of any one of the preceding embodiments.

Embodiment 80. A method of preventing or treating cancer, comprising: i) identifying a subject candidate as the subject in need of the treatment when that at least one oncogenic variant of an ErbB receptor is present in a biological sample from the subject; and ii) administering to the subject in need of the treatment a therapeutically effective amount of the morphic form of any one of the preceding embodiments.

Embodiment 81. A method of preventing or treating cancer, comprising: i) identifying a subject candidate as the subject in need of the treatment when that at least one oncogenic variant of an ErbB receptor is present in a biological sample from the subject; and ii) administering to the subject in need of the treatment the pharmaceutical composition of any one of the preceding embodiments.

Embodiment 82. A method of preventing or treating cancer, comprising administering to the subject in need thereof a therapeutically effective amount of the morphic form of any one of the preceding embodiments when that at least one oncogenic variant of an ErbB receptor is identified as being present in the subject.

Embodiment 83. A method of preventing or treating cancer, comprising administering to the subject in need thereof the morphic form of any one of the preceding embodiments when that at least one oncogenic variant of an ErbB receptor is identified as being present in the subject.

Embodiment 84. A method of preventing or treating cancer, comprising administering to the subject in need thereof a therapeutically effective amount of the morphic form of any one of the preceding embodiments when that at least one oncogenic variant of an ErbB receptor is identified as being present in a biological sample from the subject.

Embodiment 85. A method of preventing or treating cancer, comprising administering to the subject in need thereof the pharmaceutical composition of any one of the preceding embodiments when that at least one oncogenic variant of an ErbB receptor is identified as being present in a biological sample from the subject.

Embodiment 86. The morphic form of any one of the preceding embodiments for use in the inhibition of an oncogenic variant of an ErbB receptor.

Embodiment 87. The pharmaceutical composition of any one of the preceding embodiments for use in the inhibition of an oncogenic variant of an ErbB receptor.

Embodiment 88. The morphic form of any one of the preceding embodiments for use in the prevention or treatment of cancer.

Embodiment 89. The pharmaceutical composition of any one of the preceding embodiments for use in the prevention or treatment of cancer.

Embodiment 90. The morphic form of any one of the preceding embodiments for use in the prevention or treatment of cancer in a subject, wherein at least one oncogenic variant of an ErbB receptor is present in the subject.

Embodiment 91. The pharmaceutical composition of any one of the preceding embodiments for use in the prevention or treatment of cancer in a subject, wherein at least one oncogenic variant of an ErbB receptor is present in the subject.

Embodiment 92. The morphic form of any one of the preceding embodiments for use in the prevention or treatment of cancer in a subject, wherein at least one oncogenic variant of an ErbB receptor is present in a biological sample from the subject.

Embodiment 93. The pharmaceutical composition of any one of the preceding embodiments for use in the prevention or treatment of cancer in a subject, wherein at least one oncogenic variant of an ErbB receptor is present in a biological sample from the subject.

Embodiment 94. Use of the morphic form of any one of the preceding embodiments in the manufacture of a medicament for inhibiting an oncogenic variant of an ErbB receptor.

Embodiment 95. Use of the morphic form of any one of the preceding embodiments in the manufacture of a medicament for preventing or treating cancer.

Embodiment 96. The morphic form, pharmaceutical composition, method, or use of any one of the preceding embodiments, wherein the cancer is a solid tumor.

Embodiment 97. The morphic form, pharmaceutical composition, method, or use of any one of the preceding embodiments, wherein the cancer is a bladder cancer, a breast cancer, a cervical cancer, a colorectal cancer, an endometrial cancer, a gastric cancer, a glioblastoma (GBM), a head and neck cancer, a lung cancer, a non-small cell lung cancer (NSCLC), or any subtype thereof.

Embodiment 98. The morphic form, pharmaceutical composition, method, or use of any one of the preceding embodiments, wherein the cancer is glioblastoma (GBM) or any subtype thereof.

Embodiment 99. The morphic form, pharmaceutical composition, method, or use of any one of the preceding embodiments, wherein the cancer is glioblastoma.

Embodiment 100. The morphic form, pharmaceutical composition, method, or use of any one of the preceding embodiments, wherein the cancer or a tumor or a cell thereof expresses an oncogenic variant of an epidermal growth factor receptor (EGFR).

Embodiment 101. The morphic form, pharmaceutical composition, method, or use of any one of the preceding embodiments, wherein the oncogenic variant is an oncogenic variant in an ErbB receptor.

Embodiment 102. The morphic form, pharmaceutical composition, method, or use of any one of the preceding embodiments, wherein the oncogenic variant in the ErbB receptor is an allosteric variant.

Embodiment 103. The morphic form, pharmaceutical composition, method, or use of any one of the preceding embodiments, wherein the oncogenic variant is an oncogenic variant in an epidermal growth factor receptor (EGFR).

Embodiment 104. The morphic form, pharmaceutical composition, method, or use of any one of the preceding embodiments, wherein the oncogenic variant in the EGFR is an allosteric variant.

Embodiment 105. The morphic form, pharmaceutical composition, method, or use of any one of the preceding embodiments, wherein the oncogenic variant is an oncogenic variant of a HER2 receptor.

Embodiment 106. The morphic form, pharmaceutical composition, method, or use of any one of the preceding embodiments, wherein the oncogenic variant in the HER2 receptor is an allosteric variant.

Embodiment 107. The morphic form, pharmaceutical composition, method, or use of any one of the preceding embodiments, wherein the oncogenic variant is an oncogenic variant in a HER-4 receptor.

Embodiment 108. The morphic form, pharmaceutical composition, method, or use of any one of the preceding embodiments, wherein the subject or the cancer is insensitive or resistant to treatment with one or more of gefinitinib, erlotinib, afatinib, osimertinib, and necitunumab.

Embodiment 109. The morphic form, pharmaceutical composition, method, or use of any one of the preceding embodiments, wherein the sequence encoding the oncogenic variant of the EGFR comprises a deletion of exon 20 or a portion thereof and wherein the cancer, tumor or cell thereof does not comprise an oncogenic variation in a sequence encoding one or more of an EGFR kinase domain (KD), BRAF, NTRK, and KRAS or wherein.

Embodiment 110. The morphic form, pharmaceutical composition, method, or use of any one of the preceding embodiments, wherein the sequence encoding the oncogenic variant of the EGFR comprises a deletion of exon 20 or a portion thereof and wherein the cancer, tumor or cell thereof does not comprise a marker indicating responsiveness to immunotherapy.

Embodiment 111. The morphic form, pharmaceutical composition, method, or use of any one of the preceding embodiments, wherein the oncogenic variant or the oncogenic mutation is detected by a Food and Drug Administration (FDA)-approved diagnosis.

Embodiment 112. The morphic form, pharmaceutical composition, method, or use of any one of the preceding embodiments, wherein the subject has an adverse reaction to treatment with a Type I inhibitor.

Embodiment 113. The morphic form, pharmaceutical composition, method, or use of any one of the preceding embodiments, wherein the subject has an adverse reaction to treatment with one or more of gefinitnib, erlotinib, afatinib, osimertinib, necitunumab, crizotinib, alectinib, ceritinib, dabrafenib, trametinib, afatinib, sapitinib, dacomitinib, canertinib, pelitinib, WZ4002, WZ8040, WZ3146, CO-1686 and AZD9291.

### EXAMPLES

It is understood that the experimental values described in the present application are approximate and subject to instrumental variations.

X-ray Powder Diffractometer (XRPD): XRPD analysis was performed using a Bruker D8 Advance X-ray powder diffractometer. Analysis was performed using parameters as set forth below.

XRPD method 1

| | |
|---|---|
| Detector | LYNXEYE_XE_T(1D mode) |
| Open angle | 2.94° |
| Scan mode | Continuous PSD fast |
| Radiation | Cu/K-Alpha1 (λ = 1.5418 Å) |
| X-ray generator | 40 kV, 40 mA |
| power | |
| Step size | 0.02° |
| Time per step | 0.12 second per step |
| Scan range | 3° to 40° |
| Primary beam | Twin_Primary motorized slit 10.0 mm by sample |
| path slits | length; SollerMount axial soller 2.5° |
| Secondary beam | Detector OpticsMount soller slit 2.5°; |
| path slits | Twin_Secondary motorized slit 5.2 mm |
| Sample rotation | 15 rpm |
| speed | |

XRPD method 2

| | |
|---|---|
| Detector | LYNXEYE_XE_T(1D mode) |
| Open angle | 2.94° |
| Scan mode | Continuous PSD fast |
| Radiation | Cu/K-Alpha1 (λ = 1.5418 Å) |
| X-ray generator | 40 kV, 40 mA |
| power | |
| Step size | 0.02° |
| Time per step | 0.3 second per step |
| Scan range | 2° to 40° |
| Primary beam | Twin_Primary motorized slit 10.0 mm by sample |
| path slits | length; SollerMount axial soller 2.5° |
| Secondary beam | Detector OpticsMount soller slit 2.5°; |
| path slits | Twin_Secondary motorized slit 5.2 mm |
| Sample rotation | 15 rpm |
| speed | |

Differential Scanning Calorimetric (DSC): DSC analysis was performed using TA Discover 2500. Analysis was performed using parameters as set forth below.

| Sample pan | Tzero pan and Tzero hermetic lid with a pin hole | |
|---|---|---|
| Temperature range | 30 to 250° C. | |
| Heating rate | 10° | C./min |
| Nitrogen flow | 50 | mL/min |
| Sample mass | ~1-2 | mg |

Thermal Gravimetric Analysis (TGA): TGA analysis was performed using Discover 5500. Analysis was performed using parameters as set forth below.

| Sample pan | Aluminum, open | |
|---|---|---|
| Nitrogen flow | Balance 10 mL/min; sample 25 mL/min | |
| Start temperature | Ambient condition (below 35° C.) | |
| Final temperature | 300° C. (The weight loss of the compound | |
| | is no more than 20% (w/w)) | |
| Heating rate | 10° | C./min |
| Sample mass | ~2-10 | mg |

Dynamic Vapor Sorption (DVS): DVS analysis was performed using Intrinsic. Analysis was performed using parameters as set forth below.

Method 1 (for Form C)

| | | |
|---|---|---|
| Total gas flow | 200 | sccm |
| Oven temperature | 25° | C. |

| | |
|---|---|
| Solvent | Water |
| Method | Cycle: 40-0-95-0-40% RH |
| | Stage Step: 10% |
| | Equilibrium: 0.002 dm/dt (%/min ) |
| | Minimum dm/dt stability duration: 60 min |
| | Maximum dm/dt stage time: 360 min |

Method 2 (for Form F)

| | | |
|---|---|---|
| Total gas flow | 200 | sccm |
| Oven temperature | 25° | C. |

| | |
|---|---|
| Solvent | Water |
| Method | Cycle: 40-95-0-95-40% RH |
| | Stage Step: 10% |
| | Equilibrium: 0.002 dm/dt (%/min) |
| | Minimum dm/dt stability duration: 60 min |
| | Maximum dm/dt stage time: 360 min |

Karl Fischer Analysis: Karl Fischer analysis was performed using Mettler Toledo Coulometric KF Titrator C30 using the coulometric method.

Polarized Light Microscopy (PLMS: PLM analysis was performed using BX53LED OLYMPUS using a crossed polarizer with silicone oil added.

Nuclear Magnetic Resonance (NMR): NMR analysis was performed using Bruker Avance-AV 400M at a frequency of 400 MHz, a 5 mm PABBO BB-1H/D probe, 8 scanes, a temperature of 297.6 K, and a relaxation delay of 1 second.

High Performance Liquid Chrmmatograph (HPLC: HPLC analysis was performed using SHIMADZU LC-20AD/Agilent 1260 infinity 11 Binary Pump. Analysis was performed using parameters as set forth below.

| HPLC method | Wavelength: 272 nm | | |
|---|---|---|---|
| | Column: PDS-HPLC-010 | | |
| | Detector: DAD | | |
| | Column temperature: 50° C. | | |
| | Flow rate: 1.0 mL/min | | |
| | Mobile phase A: 375 μL TFA in 1 L water | | |
| | Mobile phase B: 187.5 μL TFA in 1 L ACN | | |
| | Injection volume: 5 μL | | |
| | Gradient: | | |
| | | Mobile | Mobile |
| | Time | Phase | Phase |
| | (min) | A (%) | B (%) |
| | 0.01 | 95 | 5 |
| | 1.0 | 95 | 5 |
| | 6.0 | 20 | 80 |
| | 8.0 | 20 | 80 |
| | 8.1 | 95 | 5 |
| | 9.9 | 95 | 5 |
| | 10.0 | | stop |

### Example 1. Preparation of Crystalline Forms

A crystalline form of the present disclosure may be prepared according to one or more of the protocols as described herein.

Protocol 1: Equilibration with solvents at 25° C. for 2 weeks. About 20 mg of Form A was equilibrated in a suitable amount of a solvent at 25° C. for 2 weeks with a stirring plate. The obtained suspension were filtered.

Protocol 2: Equilibration with solvents at 50° C. for 1 week. About 30 mg of Form A was equilibrated in a suitable amount of a solvent at 50° C. for 1 week with a stirring plate. The obtained suspension were filtered.

Protocol 3: Equilibration with solvents under a temperature cycle. About 30 mg of Form A was equilibrated in a suitable amount of a solvent under a temperature cycle between 5° C. to 50° C. at a heating/cooling rate of 0.2° C./min for 10 cycles.

Protocol 4: Crystallization at room temperature by slow evaporation. About 10 mg of Form A was dissolved in a suitable amount of a solvent. The obtained solution was filtered by 0.45 μm nylon filter. The filtered solution was then slowly evaporated under ambient condition.

Protocol 5: Crystallization under nitrogen flow by fast evaporation. About 10 mg of Form A was dissolved in a suitable amount of a solvent. The obtained solution was filtered by 0.45 μm nylon filter. The filtered solution was then fast evaporated under nitrogen flow.

Protocol 6: Crystallization from hot saturated solutions by slow cooling. About 20 mg of Form A was dissolved in the minimal amount of a selected solvent at 50° C. The obtained solution was filtered by 0.45 μm nylon filter. The filtered solution was then cooled to 5° C. at 0.1° C./min.

Precipitates were collected by filtration.

Protocol 7: Crystallization from hot saturated solutions by fast cooling. About 20 mg of Form A was dissolved in a minimal amount of a selected solvent at 50° C. The obtained solution was filtered by 0.45 μm nylon filter. The filtered solution were then put at 5° C. and stirred at a rate of 400 rpm. Precipitates were collected by filtration.

Protocol 8: Precipitation by addition of anti-solvent. About 20 mg of Form A was dissolved in a minimal amount of a good solvent. The solutions were filtered. Anti-solvent was added into the filtered solution slowly. Precipitates were collected by filtration. When cloudy sample were obtained from an anti-solvent experiment, slow evaporation experiments were conducted at 25° C.

Protocol 9: Variable relative humidity experiment. Form F was investigated by variable humidity XRPD experiments. Form F converted to a new crystalline form, assigned as Form L, in 0% RH. Form L reverted to Form F in >10% RH. This suggests that the Form L is a metastable anhydrate and is only stable in about 0%,6 RH. See Example 8 for more details.

Preparation of crystalline forms. Forms of the present disclosure may be prepared by one or more of the protocols as shown in Table A below.

TABLE A

| Form | Protocol |
|---|---|
| A | Compound No. 1 may be prepared according to a previously |
| | disclosed method (e.g., WO2021030711, the contents of which are |
| | hereby incorporated by reference). The crude material was purified |
| | by silica gel column chromatography, eluting with |
| | dichloromethane and methanol (1:0 to 10:1 by volume). The |
| | fractions containing pure product were combined and evaporated |
| | to give Form A as a light yellow solid. |
| B | Protocol 1: Equilibration in MeOH at 25° C. |
| C | Protocol 1: Equilibration in EtOH, acetone, MeCN, EA, MIBK, 2- |
| | Me THF, THF/heptane (v:v = 1:1), or DCM/heptane (v:v = 1:1) at |
| | 25° C. |
| | Protocol 2: Equilibration EtOH, acetone, MeCN, EA, MIBK, 2- |
| | Me THF, THF/heptane (v:v = 1:1), THF/H2O (v:v = 1:1), or |
| | acetone/H₂O (v:v = 90:10) at 50° C. |
| | Protocol 3: Temperature cycling experiments in MeOH, EtOH, |
| | acetone, ACN, EA, MIBK, 2-Me THF, THF/heptane (v:v = |
| | 1:1), or acetone/H₂O (v:v = 90:10) |
| | Protocol 4: Slow evaporation in acetone and 2-Me THF |
| | Protocol 6: Slow cooling in EA and MIBK; |
| | Protocol 7: Fast cooling in MIBK |
| | Protocol 8: Anti-solvent in THF and DCM |
| D | Protocol 1: Equilibration in 1,4-dioxane at 25° C. |
| | Protocol 2: Equilibration in 1,4-dioxane 50° C. |
| | Protocol 3: Temperature cycling experiments in 1,4-dioxane |
| | Protocol 6: Slow cooling in 1,4-dioxane |
| | Protocol 7: Fast cooing in 1,4-dioxane |
| E | Protocol 1: Equilibration in toluene at 25° C. and 50° C. |
| | Protocol 2: Equilibration in toluene at and 50° C. |
| | Protocol 3: Temperature cycling experiments in toluene |
| F | Protocol 1: Equilibration in DMSO/H₂O (v:v = 1:1), |
| | Methanol/H₂O (v:v = 70:30), or MeCN/H₂O (v:v = 90:10) |
| | at 25° C. |
| | Protocol 2: Equilibration in DMSO/H2O (v:v = 1:1), |
| | Methanol/H₂O (v:v = 70:30), MeCN/H₂O (v:v = 90:10) |
| | at 50° C. |
| | Protocol 3: Temperature cycling experiments in THF/H₂O |
| | (v:v = 1:1), DMSO/H₂O (v:v = 1:1), Methanol/H₂O (v:v = 70:30), |
| | MeCN/H₂O (v:v = 90:10) |
| | Protocol 4: Slow evaporation in MeCN, EA, toluene and THF/H₂O |
| | (v:v = 95:5) |
| | Protocol 5: Fast evaporation in MeCN |
| G | Protocol 2: Equilibration in MeOH at 50° C. |
| H | Protocol 5: Fast evaporation in 1,4-dioxane |
| I | Protocol 4: Slow evaporation in MeOH |
| J | Protocol 4: Slow evaporation in THF or 1,4-dioxane |
| K | Protocol 3: Temperature cycling experimentts in EtOH |
| L | Protocol 9: humidity XRPD experiment below 10% RH at 25° C. |

### Example 2. Preparation of Anhydrate Form C

Preparation of Form C seed crystals. About 20 mg of Form A of Compound No. 1 was equilibrated in suitable amount of solvent at 25° C. for 2 weeks with a stirring plate. Obtained suspension were filtered.

Preparation of anhydrate Form C. About 300 mg of Form A of Compound No. 1 was weighed into a 40 mL bottle. 3 mL of acetone was added into the bottle. The obtained suspension was stirred at 25° C. at a rate of 300 rpm. About 3 mg of Form C seeds was added into the suspension. Another 8 mL of acetone was added into the bottle after precipitates formed. This suspension was kept stirring at 25° C. at a rate of 400 rpm for 48 h. The obtained suspension was taken out and solids were separated out by centrifuged. The solids were dried under vacuum at 30° C. for 1 h. About 240 mg of free base Form C, was obtained as brown powders in a yield of 80%. This batch showed high crystallinity (see, e.g., FIG. 3).

### Example 3. Preparation of Hydrate Form F

Preparation of Form F seed crystals. About 30 mg of Form A of Compound No. 1 was equilibrated in a suitable amount of MeOH/H₂O (v:v=7:3) under a temperature cycle between 5° C. to 50° C. at a heating/cooling rate of 0.2° C./min for 10 cycles.

Preparation of anhydrate Form F. About 300 mg of Form A of Compound No. 1 was added into a 20 mL bottle. Then 2 mL of MeOH:H₂O (v:v=7:3) was added into the bottle. The obtained suspension was stirred at 25° C. at a rate of 400 rpm. About 3 mg of Form F seeds was added into the suspension. Another 1.6 mL of MeOH:H₂O (v:v=7:3) was added into the bottle after precipitates formed. This suspension was kept stirring at 25° C. at a rate of 400 rpm for 1 week. The obtained suspension was taken out and the solids were separated out by centrifugation.

The solids were dried under vacuum at 25° C. for 14 h. About 175 mg of Form F, was obtained as a brown powder in a yield of 58%, and characterized accordingly by XRPD (FIG. 6).

### Example 4. Competitive Equilibration Experiments

Competitive equilibration experiments were conducted to determine thermodynamic relationships of anhydrate polymorphs. The experiments were conducted in 5 solvent systems at 25° C. as shown below in Table B.

TABLE B

| Form | Solvent | Comments |
|---|---|---|
| Form B and Form C | MeCN | Form C was obtained |
| Form B and Form C | Acetone | Form C was obtained |
| Form E and Form C | EtOH | Form C was obtained |
| Form E and Form C | EA | Form C was obtained |
| Form E and Form C | Toluene | Form E was obtained |
| Form B, Form C and | MeOH | Form G was obtained |
| Form J | | |

### Example 5. Bulk Stability

Anhydrate Form C and hydrate Form F were placed under three accelerated conditions for two weeks and four weeks. Solids obtained after bulk stability study were characterized by XRPD and HPLC. As shown by Table C-i below, Form C and Form F do not change forms under various storage conditions and times.

TABLE C-1

| | |
|---|---|
| Form C | Form F |
| Purity | Purity |

Solid state, 25° C./92.5% RH in an open vial, 2 weeks

| | | |
|---|---|---|
| Bulk (HPLC) | 96.6% | 95.5% |
| Bulk (XRPD) | No form change | No form change |

Solid state, 40° C./75% RH in an open vial, 2 weeks

| | | |
|---|---|---|
| Bulk (HPLC) | 95.5% | 94.3% |
| Bulk (XRPD) | No form change | No form change |

Solid state, 60° C., tight container, 2 weeks

| | | |
|---|---|---|
| Bulk (HPLC) | 95.5% | 90.1% |
| Bulk (XRPD) | No form change | No form change |

Solid state, 25° C./92.5% RH in an open vial, 4 weeks

| | | |
|---|---|---|
| Bulk (HPLC) | 96.4% | 94.1% |
| Bulk (XRPD) | No form change | No form change |

Solid state, 40° C./75% RH in an open vial, 4 week

| | | |
|---|---|---|
| Bulk (HPLC) | 94.6% | 91.6% |
| Bulk (XRPD) | No form change | No form change |

Solid state, 60° C., tight container, 4 weeks

| | | |
|---|---|---|
| Bulk (HPLC) | 93.3% | 82.6% |
| Bulk (XRPD) | No form change | No form change |

Anhydrate Form C was analyzed for for stability after one month, as shown below in Table C-2.

TABLE C

| | | | 1 Month | | |
|---|---|---|---|---|---|
| | | | | 25° C./ | 40° C./ |
| Test item | | Initial | 5° C. | 60% RH | 75% RH |
| Appearance | | Almost | Almost | Almost | Almost |
| | | white | white | white | white |
| | | solid | solid | solid | solid |
| HPLC area Purity | | 98.1 | 98.2% | 98.2 | 98.2% |
| impurity | RRT0.967 | 0.09% | 0.07% | 0.07% | 0.08% |
| | RRT0.971 | 0.06% | 0.08% | 0.08% | 0.08% |
| | RrRT1.12 | 0.05% | — | — | — |
| | RRT1.20 | 0.06% | 0.06% | 0.06% | 0,07% |
| | RRT1.24 | 1.4% | 1.4% | 1.4% | 1.4% |
| | RRT1.26 | 0.13% | 0.13% | 0.13% | 0.13% |
| | RRT1.28 | 0.12% | 0.11% | 0.11% | 0.11% |

| | | | | |
|---|---|---|---|---|
| Water content | 0.4% | 0.5% | 0.4% | 0.7% |
| polymorph | Form C | NT | NT | NT |

“NT” means the parameter was not tested;
“—” means the value was below the report level

### Example 6. Solubility Study

2.01 mg of the free base Form C (equal to 2 mg free base after correction for water content) was weighed into 2 mL vial. 2.09 mg of the free base Form F (equal to 2 mg free base after correction for water content) was weighed into a 2 mL vial. I mL of aqueous media was added, respectively. These suspensions were stirred at 37° C. at a rate of 400 rpm. These suspensions were taken out after 2 h and 24 h, and then centrifuged at 14,000 rpm for 5 minutes. The supernatants were analyzed by HPLC. The pH of the supernatants were tested.

As shown in Table D, Form C and Form F showed pH dependent solubility and similar solubility profile in these aqueous media. They showed good solubility (>2 mg/mL) in pH 1.0 HCl solution, pH 4.5 acetate buffer (50 mM) and in SGF. They showed low solubility (about 20 μg/mL) in pH 6.8 phosphate buffer (50 mM) and (<10 μg/mL) in pure water. Both of them degraded in FeSSIF-v1 and FaSSIF-v1.

TABLE D

| | Form C | | | | | Form F | | | | |
|---|---|---|---|---|---|---|---|---|---|---|
| | | | 24 h | | | | | 24 h | | |
| | 2 h | | Solubility | | | 2 h | | Solubility | | |
| | Solubility | | (pH) | | XRPD | Solubility | | (pH) | | XRPD |
| pH 1.0 | >2 | mg/mL | >2 | mg/mL | | >2 | mg/mL | >2 | mg/mL | |
| (0.1N | | | pH 1.0 | | | | | pH 0.9 | | |
| HCl) | | | | | | | | | | |
| pH 4.5 | 2.0 | mg/mL* | >2 | mg/mL | | >2 | mg/mL | >2 | mg/mL | |
| (50 mM) | | | pH 4.5 | | | | | pH 4.6 | | |
| acetate | | | | | | | | | | |
| buffer | | | | | | | | | | |
| pH 6.8 | 20.4 | μg/mL | 16.5 | μg/mL | Form C + | 20.7 | μg/mL | 19.8 | μg/mL | Form F |
| (50 mM) | | | pH 6.7 | | Form F + | | | pH 6.7 | | |
| phosphate | | | | | inorganic | | | | | |
| buffer | | | | | salt | | | | | |
| FeSSIF- | Degradation | | Degradation | | | Degradation | | Degradation | | |
| v1, pH 5.0 | | | | | | | | | | |
| FaSSIF- | Degradation | | Degradation | | Form C + | Degradation | | Degradation | | Form F + |
| v1, pH 6.5 | | | | | NaCl | | | | | NaCl |
| SGF, | >2 | mg/mL | >2 | mg/mL | | >2 | mg/mL | >2 | mg/mL | |
| pH 2.0 | | | pH 3.7 | | | | | pH 3.5 | | |
| Water | 1.6 | μg/mL | 2.0 | μg/mL | Form C + | 7.0 | μg/mL | 7.5 | μg/mL | Form F |

| | | |
|---|---|---|
| pH 7.4 | Form F | pH 7.0 |

*the sample was cloudy in pH 4.5 acetate buffer.

### Example 7. Hygroscopicity Study of Form C and Form F

The hygroscopicity for Form C and Form F was examined. See, e.g., Table E.

TABLE E
Hygroscopicity by DVS at 25° C. dm/dt = 0.002%

| | Free base Form C (anhydrate) | | | | Free base Form F (hydrate) | | | |
|---|---|---|---|---|---|---|---|---|
| DVS | 1.6% water uptake at 90% RH | | | | 1.1% water uptake from 40% RH to 70% RH | | | |
| | 2.3% water uptake at 95% RH | | | | 10.2% water uptake from 40% RH to 95% RH | | | |
| Relative | Sorp. | Desorp. | Sorp. | Desorp. | Sorp. | Desorp. | Sorp. | Desorp. |
| Humidity | (%) | (%) | (%) | (%) | (%) | (%) | (%) | (%) |
| 0% | 0.2 | 0.2 | 0.0 | 0.0 | | 0.0 | 0.0 | |
| 10% | 0.2 | 0.3 | 0.1 | 0.1 | | 1.4 | 1.3 | |
| 20% | 0.3 | 0.4 | 0.2 | 0.2 | | 1.9 | 1.7 | |
| 30% | 0.4 | 0.5 | 0.3 | 0.3 | | 3.6 | 2.1 | |
| 40% | 0.4 | 0.5 | 0.3 | 0.4 | 2.5 | 6.3 | 2.8 | 6.4 |
| 50% | 0.5 | | | 0.6 | 2.8 | 8.0 | 3.2 | 7.9 |
| 60% | 0.6 | | | 1.1 | 3.2 | 9.4 | 3.7 | 9.3 |
| 70% | 0.8 | | | 1.4 | 3.6 | 10.2 | 4.2 | 10.0 |
| 80% | 1.2 | | | 1.7 | 10.4 | 11.0 | 10.4 | 10.8 |
| 90% | 1.8 | | | 2.2 | 11.6 | 11.8 | 11.4 | 11.6 |
| 95% | 2.5 | | | 2.5 | 12.7 | 12.9 | 12.2 | 12.2 |
| XRPD after | No form change | | | | No form change | | | |
| DVS test | | | | | | | | |

### Example 8. Variable Relative Humidity Experiments for Form F

Form F was investigated by variable humidity XRPD experiments. See, e.g., Table F. Form F converted to a new crystalline form, assigned as Form L, in 0% RH Form L reverted to Form F in >100% RH. This suggests that the Form L is a metastable anhydrate and is only stable in close to 0% RH.

TABLE F

| Humidity | XRPD |
|---|---|
| 40% RH | Form F |
| 70% RH | Form F |
| 90% RH (3 h) | Form F |
| 90% RH (6 h) | Form F |
| 70% RH | Form F |
| 40% RH | Form F |
| 10% RH | Form F |
| 0% RH (12 h) | Form L (Only stable in low humidity) |
| 0% RH (24 h) | Form L (Only stable in low humidity) |
| 10% RH | Form F |
| 40% RH | Form F |

### EQUIVALENTS

It is understood that the invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A morphic form of Compound No. 1: 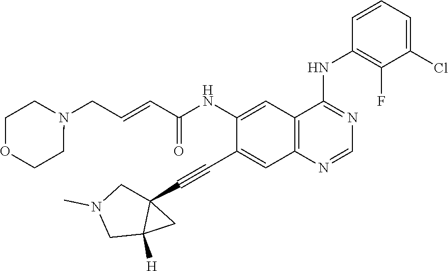 [SMILES: CN1C[C@H]2C[C@@]2(C#Cc2cc3ncnc(Nc4cccc(Cl)c4F)c3cc2NC(=O)/C=C/CN2CCOCC2)C1]
a solvate thereof, a hydrate thereof, or a pharmaceutically acceptable salt thereof.

2. The morphic form of claim 1, wherein the morphic form is a crystalline form.

3. The morphic form of claim 1 or 2, wherein the morphic form is Form A, the solvate thereof, the hydrate thereof, or the pharmaceutically acceptable salt thereof, and is characterized by an X-ray diffraction (“XRPD”) pattern comprising at least three peaks selected from 4.8±0.2, 5.6±0.2, 9.6±0.2, 17.6±0.2, 20.0±0.2, and 24.6±0.2°2θ (e.g., 4.8±0.1, 5.6±0.1, 9.6±0.1, 17.6±0.1, 20.0±0.1, and 24.6±0.1°2θ (e.g., 4.8, 5.6, 9.6, 17.6, 20.0, and 24.6°2θ)) using Cu Kα radiation, and is characterized by a DSC curve having at least one endothermic peak selected from 76±20, 141±20, 156±20, and 170±20° C. (e.g., 76±10,141±10, 156±10, and 170±10° C. (e.g., 76±5, 141±5, 156±5, and 170±5° C. (e.g., 76±4, 141±4, 156±4, and 170±4° C. (e.g., 76±3, 141±3, 156±3, and 17±3° C. (e.g., 76±2, 141±2, 156±2, and 170±2° C. (e.g., 76±1, 141±1, 156±1, and 170±1° C. (e.g., 76±0.5, 141±0.5, 156±0.5, and 170±0.5° C.))))))).

4. The morphic form of claim 1 or 2, wherein the morphic form is Form B, the solvate thereof, the hydrate thereof, or the pharmaceutically acceptable salt thereof, and is characterized by an X-ray diffraction (“XRPD”) pattern comprising at least three peaks selected from 3.2±0.2, 6.5±0.2, 13.4±0.2, 16.7±0.2, 17.7±0.2, and 18.6±0.2°2θ (e.g., 3.2±0.1, 6.5±0.1, 13.4±0.1, 16.7±0.1, 17.7±0.1, and 18.6±0.1°2θ (e.g., 3.2, 6.5, 13.4, 16.7, 17.7, and 18.6°2θ)) using Cu Kα radiation, and is characterized by a DSC curve having at least one endothermic peak selected from 155±20 and 176±20° C. (e.g., 155±10 and 176±10° C. (e.g., 155±5 and 176±5° C. (e.g., 155±4 and 176±4° C. (e.g., 155±3 and 176±3° C. (e.g., 155±2 and 176±2° C. (e.g., 155±1 and 176±1° C. (e.g., 155±0.5 and 176±0.5° C.))))))).

5. The morphic form of claim 1 or 2, wherein the morphic form is Form C, the solvate thereof, the hydrate thereof, or the pharmaceutically acceptable salt thereof, and is characterized by an X-ray diffraction (“XRPD”) pattern comprising at least three peaks selected from 4.0±0.2, 12.6±0.2, 14.1±0.2, 16.1±0.2, 16.7±0.2, and 19.1±0.2°2θ (e.g., 4.0±0.1, 12.6±0.1, 14.1±0.1, 16.1±0.1, 16.7±0.1, and 19.1±0.1°2θ (e.g., 4.0, 12.6, 14.1, 16.1, 16.7, and 19.1°2θ)) using Cu Kα radiation, and is characterized by a DSC curve having an endothermic peak at 181±20° C. (e.g., 181±10° C. (e.g., 181±5° C. (e.g., 181±4° C. (e.g., 181±3° C. (e.g., 181±2° C. (e.g., 181±1° C. (e.g., 181±0.5° C.))))))).

6. The morphic form of claim 1 or 2, wherein the morphic form is Form D, the solvate thereof, the hydrate thereof, or the pharmaceutically acceptable salt thereof, and is characterized by an X-ray diffraction (“XRPD”) pattern comprising at least three peaks selected from 5.4±0.2, 9.7±0.2, 15.3±0.2, 18.8±0.2, 19.3±0.2, and 23.5±0.2°2θ (e.g., 5.4±0.1, 9.7±0.1, 15.3±0.1, 18.8±0.1, 19.3±0.1, and 23.5±0.1°2θ (e.g., 5.4, 9.7, 15.3, 18.8, 19.3, and 23.5°2θ)) using Cu Kα radiation, and is characterized by a DSC curve having at least one endothermic peak selected from 128±20, 149±20, and 175±20° C. (e.g., 128±10, 149±10, and 175±10° C. (e.g., 128±5, 149±5, and 175±5° C. (e.g., 128±4, 149±4, and 175±4° C. (e.g., 128±3, 149±3, and 175±3° C. (e.g., 128±2, 149±2, and 175±2° C. (e.g., 128±1, 149±1, and 175±1° C. (e.g., 128±0.5, 149±0.5, and 175±0.5° C.))))))).

7. The morphic form of claim 1 or 2, wherein the morphic form is Form E, the solvate thereof, the hydrate thereof, or the pharmaceutically acceptable salt thereof, and is characterized by an X-ray diffraction (“XRPD”) pattern comprising at least three peaks selected from 7.4±0.2, 13.3±0.2, 15.8±0.2, 16.3±0.2, 19.2±0.2, and 22.4±0.2°2θ (e.g., 7.4±0.1, 13.3±0.1, 15.8±0.1, 16.3±0.1, 19.2±0.1, and 22.4±0.1°2θ (e.g., 7.4, 13.3, 15.8, 16.3, 19.2, and 22.4°2θ)) using Cu Kα radiation, and is characterized by a DSC curve having an endothermic peak at 173±20° C. (e.g., 173±10° C. (e.g., 173±5° C. (e.g., 173±4° C. (e.g., 173±3° C. (e.g., 173±2° C. (e.g., 173±1° C. (e.g., 173±0.5° C.))))))).

8. The morphic form of claim 1 or 2, wherein the morphic form is Form F, the solvate thereof, the hydrate thereof, or the pharmaceutically acceptable salt thereof, and is characterized by an X-ray diffraction (“XRPD”) pattern comprising at least three peaks selected from 5.5±0.2, 7.2±0.2, 14.5±0.2, 15.2±0.2, 16.4±0.2, and 21.9±0.2°2θ (e.g., 5.5±0.1, 7.2±0.1, 14.5±0.1, 15.2±0.1, 16.4±0.1, and 21.9±0.1°2θ (e.g., 5.5, 7.2, 14.5, 15.2, 16.4, and 21.9°2θ)) using Cu Kα radiation, and is characterized by a DSC curve having at least one endothermic peak selected from 48±20, 80±20, 146±20, and 174±20° C. (e.g., 48±10, 80±10, 146±10, and 174±10° C. (e.g., 48±5, 80 5, 146±5, and 174±5° C. (e.g., 48±4, 80±4, 146±4, and 174±4° C. (e.g., 48±3, 80±3, 146±3, and 174±3° C. (e.g., 48±2, 80±2, 146±2, and 174±2° C. (e.g., 48±1, 80±1, 146±1, and 174±1° C. (e.g., 48±0.5, 80±0.5, 146±0.5, and 174±0.5° C.))))))).

9. The morphic form of claim 1 or 2, wherein the morphic form is Form G, the solvate thereof, the hydrate thereof, or the pharmaceutically acceptable salt thereof, and is characterized by an X-ray diffraction (“XRPD”) pattern comprising at least three peaks selected from 4.0±0.2, 5.3±0.2, 7.1±0.2, 16.0±0.2, 16.7±0.2, and 19.2±0.2°2θ (e.g., 4.0±0.1, 5.3±0.1, 7.1±0.1, 16.0±0.1, 16.7±0.1, and 19.2±0.1°2θ (e.g., 4.0, 5.3, 7.1, 16.0, 16.7, and 19.2°2θ)) using Cu Kα radiation, and is characterized by a DSC curve having at least one endothermic peak selected from 34±20, 175±20, and 182±20° C. (e.g., 34±10, 175±10, and 182±10° C. (e.g., 34 5, 175±5, and 182±5° C. (e.g., 34±4, 175±4, and 182±4° C. (e.g., 34±3, 175±3, and 182±3° C. (e.g., 34±2, 175±2, and 182±2° C. (e.g., 34±1, 175±1, and 182±1° C. (e.g., 34±0.5, 175±0.5, and 182±0.5° C.))))))).

10. The morphic form of claim 1 or 2, wherein the morphic form is Form H, the solvate thereof, the hydrate thereof, or the pharmaceutically acceptable salt thereof, and is characterized by an X-ray diffraction (“XRPD”) pattern comprising at least three peaks selected from 4.6±0.2, 5.6±0.2, 8.6±0.2, 13.8±0.2, 17.0±0.2, and 17.8±0.2°2θ (e.g., 4.6±0.1, 5.6±0.1, 8.6±0.1, 13.8±0.1, 17.0±0.1, and 17.8±0.1°2θ (e.g., 4.6, 5.6, 8.6, 13.8, 17.0, and 17.8°2θ)) using Cu Kα radiation, and is characterized by a DSC curve having at least one endothermic peak selected from 62±20 and 153±20° C. (e.g., 62±10 and 153±10° C. (e.g., 62±5 and 153±5° C. (e.g., 62±4 and 153±4° C. (e.g., 62±3 and 153±3° C. (e.g., 62±2 and 153±2° C. (e.g., 62±1 and 153±1° C. (e.g., 62±0.5 and 153±0.5° C.))))))).

11. The morphic form of claim 1 or 2, wherein the morphic form is Form I, the solvate thereof, the hydrate thereof, or the pharmaceutically acceptable salt thereof, and is characterized by an X-ray diffraction (“XRPD”) pattern comprising at least three peaks selected from 5.1±0.2, 17.0±0.2, 20.4±0.2, 21.5±0.2, 22.3±0.2, and 25.5±0.2°2θ (e.g., 5.1±0.1, 17.0±0.1, 20.4±0.1, 21.5±0.1, 22.3±0.1, and 25.5±0.1°2θ (e.g., 5.1, 17.0, 20.4, 21.5, 22.3, and 25.5°2θ)) using Cu Kα radiation.

12. The morphic form of claim 1 or 2, wherein the morphic form is Form J, the solvate thereof, the hydrate thereof, or the pharmaceutically acceptable salt thereof, and is characterized by an X-ray diffraction (“XRPD”) pattern comprising at least three peaks selected from 4.5±0.2, 5.1±0.2, 10.4±0.2, 16.0±0.2, 17.9±0.2, and 22.5±0.2°2θ (e.g., 4.5±0.1, 5.1±0.1, 10.4±0.1, 16.0±0.1, 17.9±0.1, and 22.5±0.1°2θ (e.g., 4.5, 5.1, 10.4, 16.0, 17.9, and 22.5°2θ)) using Cu Kα radiation, and is characterized by a DSC curve having at least one endothermic peak selected from 137±20 and 166±20° C. (e.g., 137±10 and 166±10° C. (e.g., 137±5 and 166±5° C. (e.g., 137±4 and 166±4° C. (e.g., 137±3 and 166±3° C. (e.g., 137±2 and 166±2° C. (e.g., 137±1 land 166±1° C. (e.g., 137±0.5 and 166±0.5° C.))))))).

13. The morphic form of claim 1 or 2, wherein the morphic form is Form K, the solvate thereof, the hydrate thereof, or the pharmaceutically acceptable salt thereof, and is characterized by an X-ray diffraction (“XRPD”) pattern comprising at least three peaks selected from 5.2±0.2, 6.8±0.2, 17.0±0.2, 20.5±0.2, 21.6±0.2, and 22.4±0.2°2θ (e.g., 5.2±0.1, 6.8±0.1, 17.0±0.1, 20.5±0.1, 21.6±0.1, and 22.4±0.1°2θ (e.g., 5.2, 6.8, 17.0, 20.5, 21.6, and 22.4°2θ)) using Cu Kα radiation.

14. The morphic form of claim 1 or 2, wherein the morphic form is Form L, the solvate thereof, the hydrate thereof, or the pharmaceutically acceptable salt thereof, and is characterized by an X-ray diffraction (“XRPD”) pattern comprising at least three peaks selected from 5.4±0.2, 7.8±0.2, 14.8±0.2, 15.3±0.2, 16.6±0.2, and 19.3±0.2°2θ (e.g 5.4±0.1, 7.8±0.1, 14.8±0.1, 15.3±0.1, 16.6±0.1, and 19.3±0.1°2θ (e.g., 5.4, 7.8, 14.8, 15.3, 16.6, and 19.3°2θ)) using Cu Kα radiation.

15. The morphic form of any one of the preceding claims, which is at least 90, 95, 96, 97, 98, or 99% pure.

16. A pharmaceutical composition comprising a therapeutically effective amount of any one, or combination, of the morphic forms of any preceding claim and a pharmaceutically acceptable excipient.

17. A method of inhibiting an oncogenic variant of an ErbB receptor, comprising administering to the subject in need thereof a therapeutically effective amount of the morphic form, or pharmaceutical composition, of any one of the preceding claims.

18. A method of preventing or treating cancer, comprising administering to the subject in need thereof a therapeutically effective amount of the morphic form, or pharmaceutical composition, of any one of the preceding claims.

19. A method of preventing or treating cancer, comprising: i) identifying a subject candidate as the subject in need of the treatment when that at least one oncogenic variant of an ErbB receptor is present in the subject, or in a biological sample from the subject; and ii) administering to the subject in need of the treatment a therapeutically effective amount of the morphic form, or pharmaceutical composition, of any one of the preceding claims.

20. A method of preventing or treating cancer, comprising administering to the subject in need thereof the morphic form, or pharmaceutical composition, of any one of the preceding claims when that at least one oncogenic variant of an ErbB receptor is identified as being present in the subject, or in a biological sample from the subject.

21. The morphic form, or pharmaceutical composition, of any one of the preceding claims for use in the inhibition of an oncogenic variant of an ErbB receptor.

22. The morphic form, or pharmaceutical composition, of any one of the preceding claims for use in the prevention or treatment of cancer.

23. The morphic form, or pharmaceutical composition, of any one of the preceding claims for use in the prevention or treatment of cancer in a subject, wherein at least one oncogenic variant of an ErbB receptor is present in the subject, or in a biological sample from the subject.

24. Use of the morphic form of any one of the preceding claims in the manufacture of a medicament for inhibiting an oncogenic variant of an ErbB receptor.

25. Use of the morphic form of any one of the preceding claims in the manufacture of a medicament for preventing or treating cancer.

26. The morphic form, pharmaceutical composition, method, or use of any one of the preceding claims, wherein the cancer is a solid tumor, a bladder cancer, a breast cancer, a cervical cancer, a colorectal cancer, an endometrial cancer, a gastric cancer, a glioblastoma (GBM), a head and neck cancer, a lung cancer, a non-small cell lung cancer (NSCLC), or any subtype thereof.

27. The morphic form, pharmaceutical composition, method, or use of any one of the preceding claims, wherein the cancer or a tumor or a cell thereof expresses an oncogenic variant of an epidermal growth factor receptor (EGFR).

28. The morphic form, pharmaceutical composition, method, or use of any one of the preceding claims, wherein the oncogenic variant is an oncogenic variant in an ErbB receptor, epidermal growth factor receptor (EGFR), of a HER2 receptor, or a HER-4 receptor and wherein the oncogenic variant in the ErbB receptor, EGFR, or HER2 receptor is an allosteric variant.

29. The morphic form, pharmaceutical composition, method, or use of any one of the preceding claims, wherein the subject or the cancer is insensitive or resistant to treatment with one or more of gefinitinib, erlotinib, afatinib, osimertinib, and necitunumab.

30. The morphic form, pharmaceutical composition, method, or use of any one of the preceding claims, wherein the sequence encoding the oncogenic variant of the EGFR comprises a deletion of exon 20 or a portion thereof and wherein the cancer, tumor or cell thereof does not comprise an oncogenic variation in a sequence encoding one or more of an EGFR kinase domain (KD), BRAF, NTRK, and KRAS or a marker indicating responsiveness to immunotherapy.

31. The morphic form, pharmaceutical composition, method, or use of any one of the preceding claims, wherein the oncogenic variant or the oncogenic mutation is detected by a Food and Drug Administration (FDA)-approved diagnosis.

32. The morphic form, pharmaceutical composition, method, or use of any one of the preceding claims, wherein the subject has an adverse reaction to treatment with a Type I inhibitor.

33. The morphic form, pharmaceutical composition, method, or use of any one of the preceding claims, wherein the subject has an adverse reaction to treatment with one or more of gefinitinib, erlotinib, afatinib, osimertinib, necitunumab, crizotinib, alectinib, ceritinib, dabrafenib, trametinib, afatinib, sapitinib, dacomitinib, canertinib, pelitinib, WZ4002, WZ8040, WZ3146, CO-1686 and AZD9291.
